(19) United States
(12) Patent Application Publication
(10) Pub. No.: US 2024/0018104 A1
(43) Pub. Date: Jan. 18, 2024

(54) COMPOUND FOR ORGANIC ELECTRONIC ELEMENT, ORGANIC ELECTRONIC ELEMENT USING SAME, AND ELECTRONIC DEVICE THEREOF

(71) Applicant: DUK SAN NEOLUX CO., LTD., Cheonan-si, Chungcheongnam-do (KR)

(72) Inventors: Hyoung Keun PARK, Cheonan-si, Chungcheongnam-do (KR); Jung Geun LEE, Cheonan-si, Chungcheongnam-do (KR); Sun Hee LEE, Cheonan-si, Chungcheongnam-do (KR); Soung Yun MUN, Cheonan-si, Chungcheongnam-do (KR); Dae Sic KIM, Cheonan-si, Chungcheongnam-do (KR); Chi Hyun PARK, Cheonan-si, Chungcheongnam-do (KR)

(73) Assignee: DUK SAN NEOLUX CO., LTD., Cheonan-si, Chungcheongnam-do (KR)

(21) Appl. No.: 18/004,486

(22) PCT Filed: Jul. 9, 2021
(86) PCT No.: PCT/KR2021/008787

§ 371 Date: Jan. 6, 2023

(30) Foreign Application Priority Data
Jul. 9, 2020 (KR) 10-2020-0084796

(51) Int. Cl. C07D 209/86; C07D 405/12; C07D 409/12; H10K 85/60

(52) U.S. Cl. CPC C07D 209/86; C07D 405/12; H10K 85/6576; H10K 85/654; C07D 409/12

(57) ABSTRACT

Provided are a compound capable of improving the light-emitting efficiency, stability, and lifespan of an element; an organic electronic element using same; and an electronic device thereof.

### BACKGROUND

### Technical Field

The present invention relates to compound for organic electronic element, organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

In the organic light emitting diode, the most problematic is the lifespan and the efficiency. As the display becomes large, the efficiency and the lifespan problem must be solved. Efficiency, life span, driving voltage and the like are related to each other, as the efficiency is increased, the driving voltage is relatively decreased, and as the driving voltage drops, the crystallization of the organic material due to Joule heating generated during driving is reduced, and as a result, the life span tends to increase.

However, simply improving the organic material layer cannot maximize the efficiency. This is because, when the optimal combination of the energy level and T1 value between each organic material layer and the intrinsic properties (mobility, interface characteristics, etc.) of the material are achieved, long life and high efficiency can be achieved at the same time.

In a phosphorescent organic electric element using a phosphorescent dopant material, the LUMO and HOMO levels of the host material have a great influence on the efficiency and life span of the organic electric element, and depending on whether electron and hole injection in the emitting layer can be efficiently controlled, charge balance in the emitting layer, dopant quenching, and reduction in efficiency and lifespan due to light emission at the hole transport layer interface can be prevented.

For fluorescent and phosphorescent host materials, recently we have been studying the increase of efficiency and life span of organic electric elements using TADF (thermal activated delayed fluorescent), Exciplex, etc., particularly, and many studies have been carried out to identify the energy transfer method from the host material to the dopant material.

Although there are various methods for identifying the energy transfer in the emitting layer for TADF (thermally activated delayed fluorescent) and exciplex, it can be easily confirmed by the PL lifespan (TRTP) measurement method.

The TRTP (Time Resolved Transient PL) measurement method is a method of observing Decay Time after irradiating a pulsed light source onto a host thin film, and is a measurement method that can identify the energy transfer method by observing energy transfer and emission delay time. The TRTP measurement is a measurement method capable of distinguishing fluorescence and phosphorescence, and an energy transfer method in a mixed host material, an exciplex energy transfer method, and a TADF energy transfer method.

As such, there are various factors that affect the efficiency and lifespan depending on how energy is transferred from the host material to the dopant material.

As such, there are various factors that affect efficiency and lifespan depending on how energy is transferred from the host material to the dopant material, and since the energy transfer method is different depending on the material, a stable and efficient host material for an organic electric device has not been sufficiently developed. Therefore, development of new materials is continuously required, and in particular, development of a host material for the emitting layer is urgently required.

Moreover, recently in order to solve the light emitting problem in the hole transport layer in organic light emitting devices, an emitting auxiliary layer must exist between the hole transport layer and the emitting layer, and it is time to develop different emitting auxiliary layers according to each of the emitting layers (R, G, and B).

In general, electrons are transferred from the electron transport layer to the emitting layer, and holes are transferred from the hole transport layer to the emitting layer to generate excitons by recombination.

However, the material used for the hole transport layer has a low HOMO value and therefore has mostly low T1 value. As a result, the exciton generated in the emitting layer is transferred to the hole transport layer, resulting in charge unbalance in the emitting layer, and light is emitted at the interface of the hole transport layer.

When light is emitted at the interface of the hole transport layer, the color purity and efficiency of the organic electronic device are lowered and the life span is shortened. Therefore, it is urgently required to develop an emitting-auxiliary layer having a high T1 value and a HOMO level between the HOMO energy level of the hole transport layer and the HOMO energy level of the emitting layer.

Meanwhile, it is necessary to develop a hole injection layer material having stable characteristics, that is, a high glass transition temperature, against joule heating generated when the device is driven, while delaying penetration of the metal oxide from the anode electrode (ITO), which is one of the causes of shortening the lifespan of the organic electronic device, into the organic layer. The low glass transition temperature of the hole transport layer material has a characteristic that when the device is driven, the uniformity of the surface of the thin film is lowered, which has been reported to have a great influence on the lifespan of the device. In addition, OLED devices are mainly formed by a deposition method, and it is necessary to develop a material that can withstand long time in deposition, that is, a material having high heat resistance characteristics.

That is, in order to sufficiently exhibit the excellent characteristics of the organic electronic element, a material for forming an organic material layer in an element such as a hole injection material, a hole transport material, a light emitting material, an electron transport material, an electron injection material, an emitting-auxiliary layer material should be supported by stable and efficient materials. However, such a stable and efficient organic material layer material for an organic electronic element has not been sufficiently developed yet. Therefore, development of new materials is continuously required.

BRIEF DESCRIPTION OF THE INVENTION

### Summary

In order to solve the problems of the background art described above, the present invention has discovered a compound having a novel structure, and also found that when the compound is applied to an organic electronic element, the luminous efficiency, stability and lifespan of the element can be greatly improved.

Accordingly, an object of the present invention is to provide a novel compound, an organic electronic element using the same, and an electronic device thereof.

### Technical Solution

The present invention provides a compound represented by Formula 1.

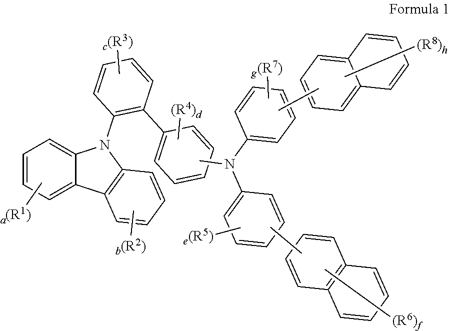

In another aspect, the present invention provides an organic electronic element and an electronic device comprising the compound represented by Formula 1.

### Effects of the Invention

By using the compound according to the present invention, it is possible to achieve a high luminous efficiency, a low driving voltage, and a high heat resistance of the element, and can greatly improve the color purity and lifespan of the element.

### BRIEF DESCRIPTION OF THE DRAWINGS

FIG. 1 to FIG. 3 illustrate an example of an organic electronic element according to the present invention.

FIG. 4 shows a Formula according to one aspect of the present invention.

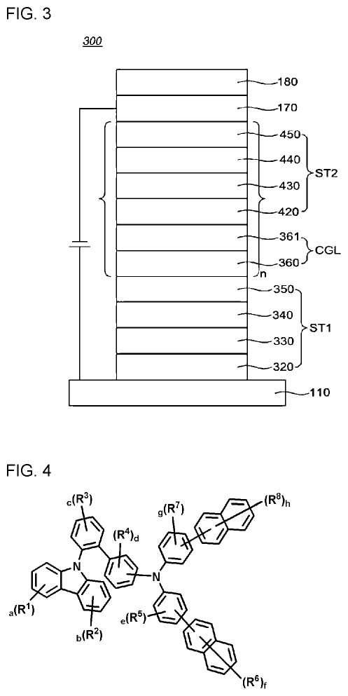

| | |
|---|---|
| 100, 200, 300: organic electronic element | 110: the first electrode |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION OF THE INVENTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected “, ” coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

Unless otherwise stated, the term “aryl group” or “arylene group”, as used herein, has 6 to 60 carbon atoms, but is not limited thereto. Herein, the aryl group or arylene group means a monocyclic and polycyclic aromatic group, and may also be formed in conjunction with an adjacent group. Examples of “aryl group” may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of monocyclic and polycyclic rings, and may include heteroaliphatic ring and/or heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO2 instead of carbon consisting of cycle. For example, “heterocyclic group” includes compound below.

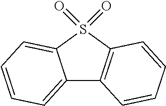 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

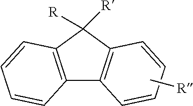

The term “Spiro compound”, as used herein, has a ‘Spiro union’, and a Spiro union means a connection in which two rings share only one atom. At this time, atoms shared in the two rings are called ‘spiro atoms’, and these compounds are called ‘monospiro’, ‘di-spiro’ and ‘tri-spiro’, respectively, depending on the number of atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include one or more heteroatoms, but are not limited thereto.

Unless otherwise stated, the term “substituted or unsubstituted”, as used herein, means that substitution is substituted by at least one substituent selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiophen group, a C₆-C₂₀ arylthiophen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited thereto.

Unless otherwise expressly stated, the Formula used in the present invention, as used herein, is applied in the same manner as the substituent definition according to the definition of the exponent of the following Formula.

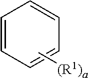

wherein, when a is an integer of zero, the substituent R¹ is absent, when a is an integer of 1, the sole substituent R¹ is linked to any one of the carbon constituting the benzene ring, when a is an integer of 2 or 3, each substituent R¹s may be the same and different, when a is an integer of 4 to 6, and is linked to the benzene ring in a similar manner, whereas the indication of hydrogen bound to the carbon forming the benzene ring is omitted.

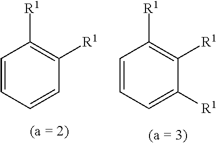

Hereinafter, a compound according to an aspect of the present invention and an organic electronic element comprising the same will be described.

The present invention provides a compound represented by Formula 1.

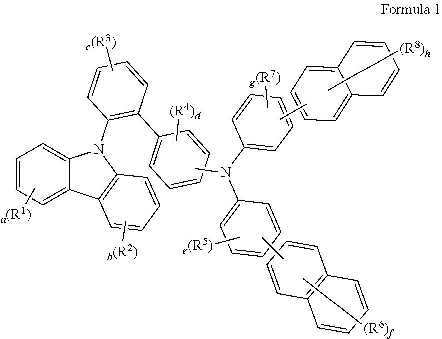

In Formula 1, each symbol may be defined as follows:

1) R¹, R², R³, R⁴, R⁵ and R⁷ are each independently the same as or different from each other, and are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂-C₆₀ heterocyclic group including at least one heteroatom of O, N, S, Si or a fused ring group of a C₃-C₆₀ aliphatic ring and a C₆-C₆₀ aromatic ring; or in case a, b, c, d, e and g are 2 or more, a plurality of adjacent R¹s, or a plurality of R²s, or a plurality of R³s, or a plurality of R⁴s, or a plurality of R⁵s, or a plurality of R⁷s may be bonded to each other to form a ring,

wherein in case R¹, R², R³, R⁴, R⁵ and R⁷ are an alkyl group, it may be preferably a C₁-C₃₀ alkyl group, and more preferably a C₁-C₂₄ alkyl group,

wherein in case R¹, R², R³, R⁴, R⁵ and R⁷ are an alkenyl group, it may be preferably a C₂-C₃₀ alkenyl group, and more preferably a C₂-C₂₄ alkenyl group,

wherein in case R¹, R², R³, R⁴, R⁵ and R⁷ are an alkynyl group, it may be preferably a C₂-C₃₀ alkynyl group, and more preferably a C₂-C₂₄ alkynyl group,

wherein in case R¹, R², R³, R⁴, R⁵ and R⁷ are an alkoxyl group, it may be preferably an C₁˜C₃₀ alkoxyl group, and more preferably an C₁˜C₂₄ alkoxyl group,

wherein in case R¹, R², R³, R⁴, R⁵ and R⁷ are an aryloxy group, it may be preferably an C₆˜C₃₀ aryloxy group, and more preferably an C₆˜C₂₄ aryloxy group,

wherein in case R¹, R², R³, R⁴, R⁵ and R⁷ are an aryl group, it may be preferably a C₆-C₃₀ aryl group, and more preferably a C₆-C₂₅ aryl group, for example, it may be phenylene, biphenyl, naphthalene, terphenyl, etc,

wherein in case R¹, R², R³, R⁴, R⁵ and R⁷ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-primido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

wherein in case R¹, R², R³, R⁴, R⁵ and R⁷ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring;

2) R⁶ and R⁸ are each independently the same as or different from each other, and are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a fused ring group of a C₃-C₆₀ aliphatic ring and a C₆-C₆₀ aromatic ring; or in case f and h are 2 or more, a plurality of adjacent R⁶ s, or a plurality of R⁸ s may be bonded to each other to form a ring,

wherein in case R⁶ and R⁸ are an alkyl group, it may be preferably a C₁-C₃₀ alkyl group, and more preferably a C₁-C₂₄ alkyl group,

wherein in case R⁶ and R⁸ are an alkenyl group, it may be preferably a C₂-C₃₀ alkenyl group, and more preferably a C₂-C₂₄ alkenyl group,

wherein in case R⁶ and R⁸ are an alkynyl group, it may be preferably a C₂-C₃₀ alkynyl group, and more preferably a C₂-C₂₄ alkynyl group,

wherein in case R⁶ and R⁸ are an alkoxyl group, it may be preferably an C₁-C₃₀ alkoxyl group, and more preferably an C₁-C₂₄ alkoxyl group,

wherein in case R⁶ and R⁸ are an aryloxy group, it may be preferably an C6-C₃₀ aryloxy group, and more preferably an C₆˜C₂₄ aryloxy group,

wherein in case R⁶ and R⁸ are an aryl group, it may be preferably a C₆-C₃₀ aryl group, and more preferably a C₆-C₂₅ aryl group, for example, it may be phenylene, biphenyl, naphthalene, terphenyl, etc,

wherein in case R⁶ and R⁸ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring;

3) a, b, c, d, e and g are independently integers from 0 to 4, h and f are independently integers from 0 to 7,

4) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; 06-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, a compound represented by Formula 1 is represented by any one of Formulas 1-1 to 1-3:

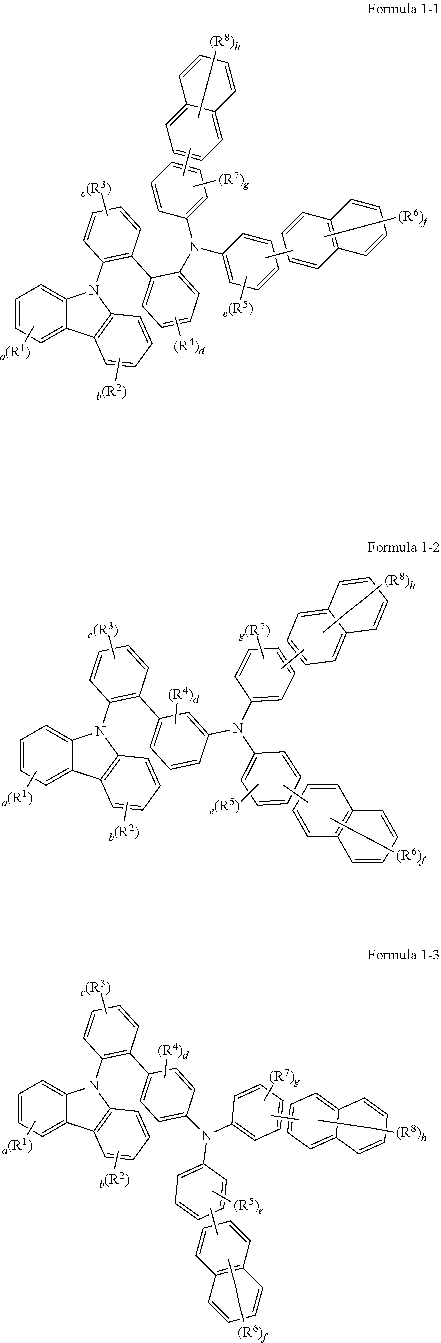

wherein R¹, R², R³, R⁴, R⁵, R⁶, R⁷, R⁸, a, b, c, d, e, f, g and h are the same as defined in Formula 1.
Also, a compound represented by Formula 1 is represented by any one of Formulas 1-4 to 1-6.

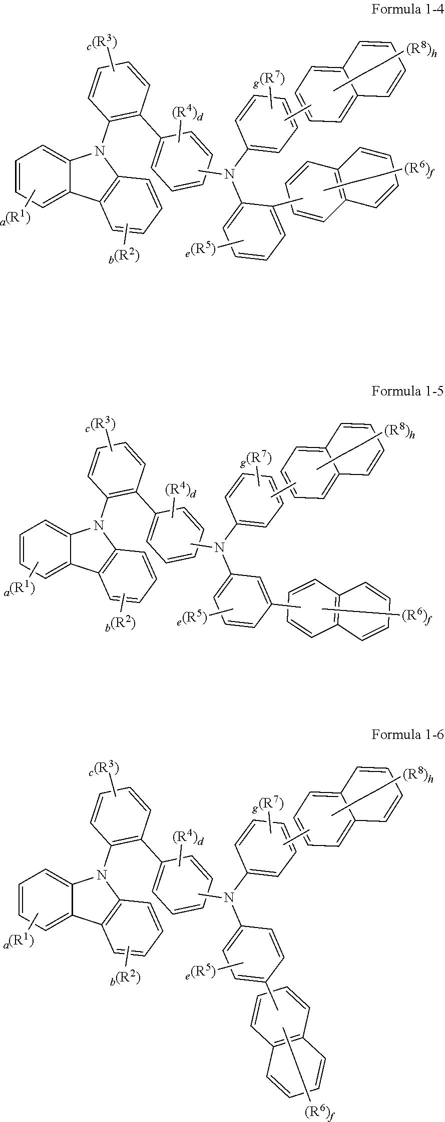

wherein R¹, R², R³, R⁴, R⁵, R⁶, R⁷, R⁸, a, b, c, d, e, f, g and h are the same as defined in Formula 1.
Also, a compound represented by Formula 1 is represented by Formula 1-7 or 1-8:

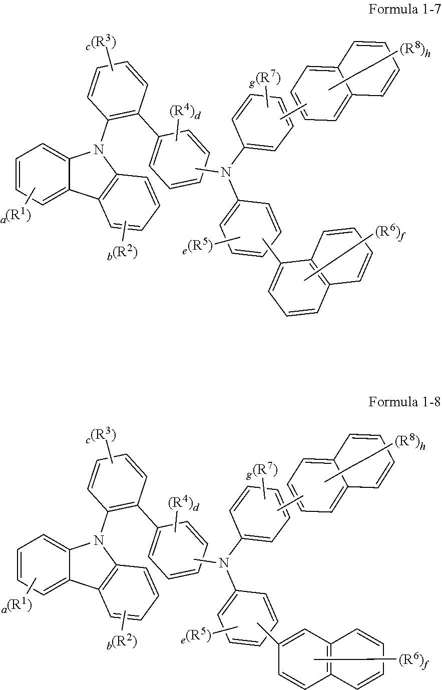

wherein R¹, R², R³, R⁴, R⁵, R⁶, R⁷, R⁸, a, b, c, d, e, f, g and h are the same as defined in Formula 1.
Also, a compound represented by Formula 1 is represented by Formula 1-9:

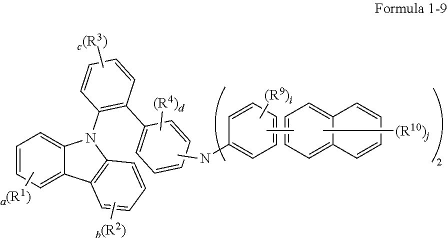

wherein
1) R¹, R², R³, R⁴, a, b, c and d are the same as defined in Formula 1,
2) R⁹ is the same as the definition of R 1 in Formula 1,
3) R¹⁰ is the same as the definition of R⁶ in Formula 1,
4) i is an integer of 0 to 4, j is an integer of 0 to 7.
Also, a compound represented by Formula 1 is represented by any one of Formulas 1-10 to 1-12:

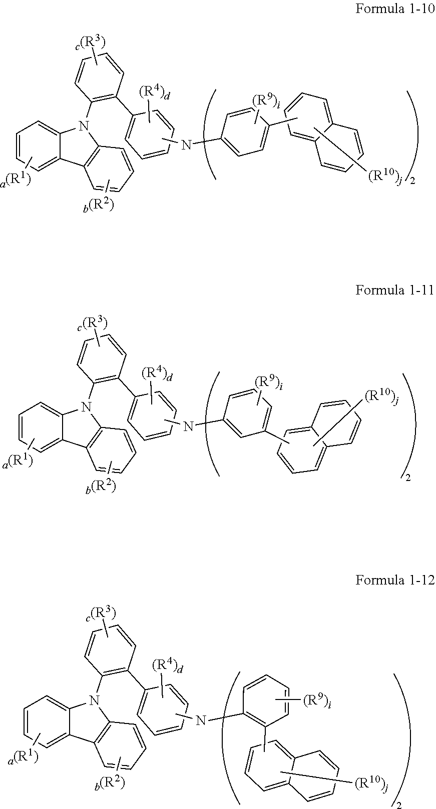

wherein:
1) R¹, R², R³, R⁴, a, b, c and d are the same as defined in Formula 1,
2) R⁹, R¹⁰, i and j are the same as defined in Formula 1-9.
Specifically, the compound represented by Formula 1 may be any one of the following compounds P-1 to P-90, but is not limited thereto:

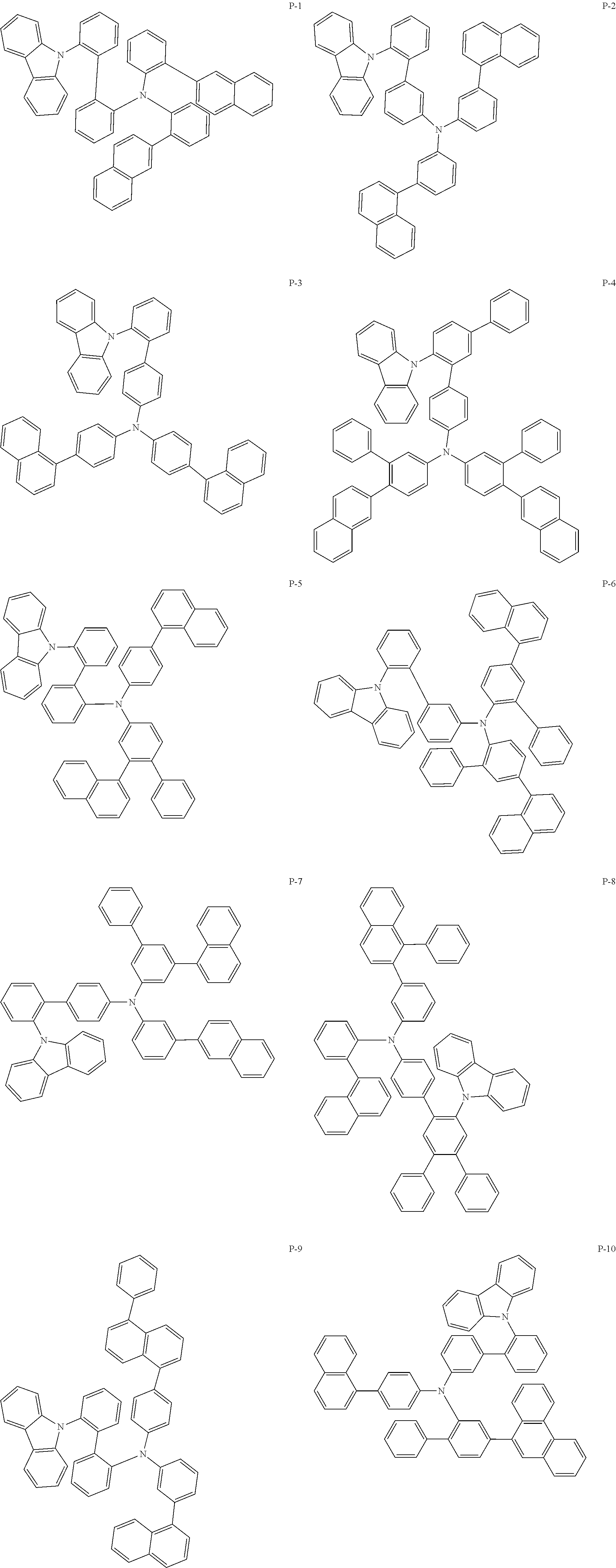

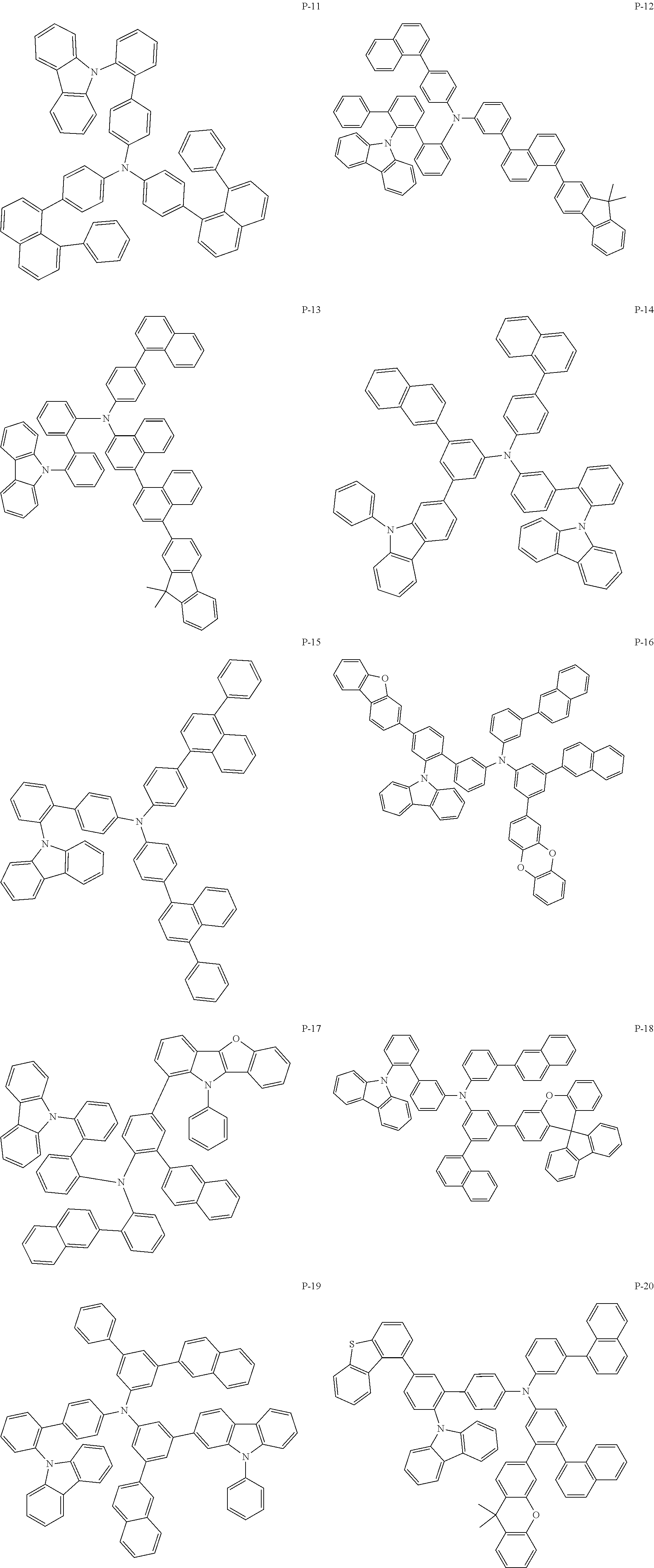

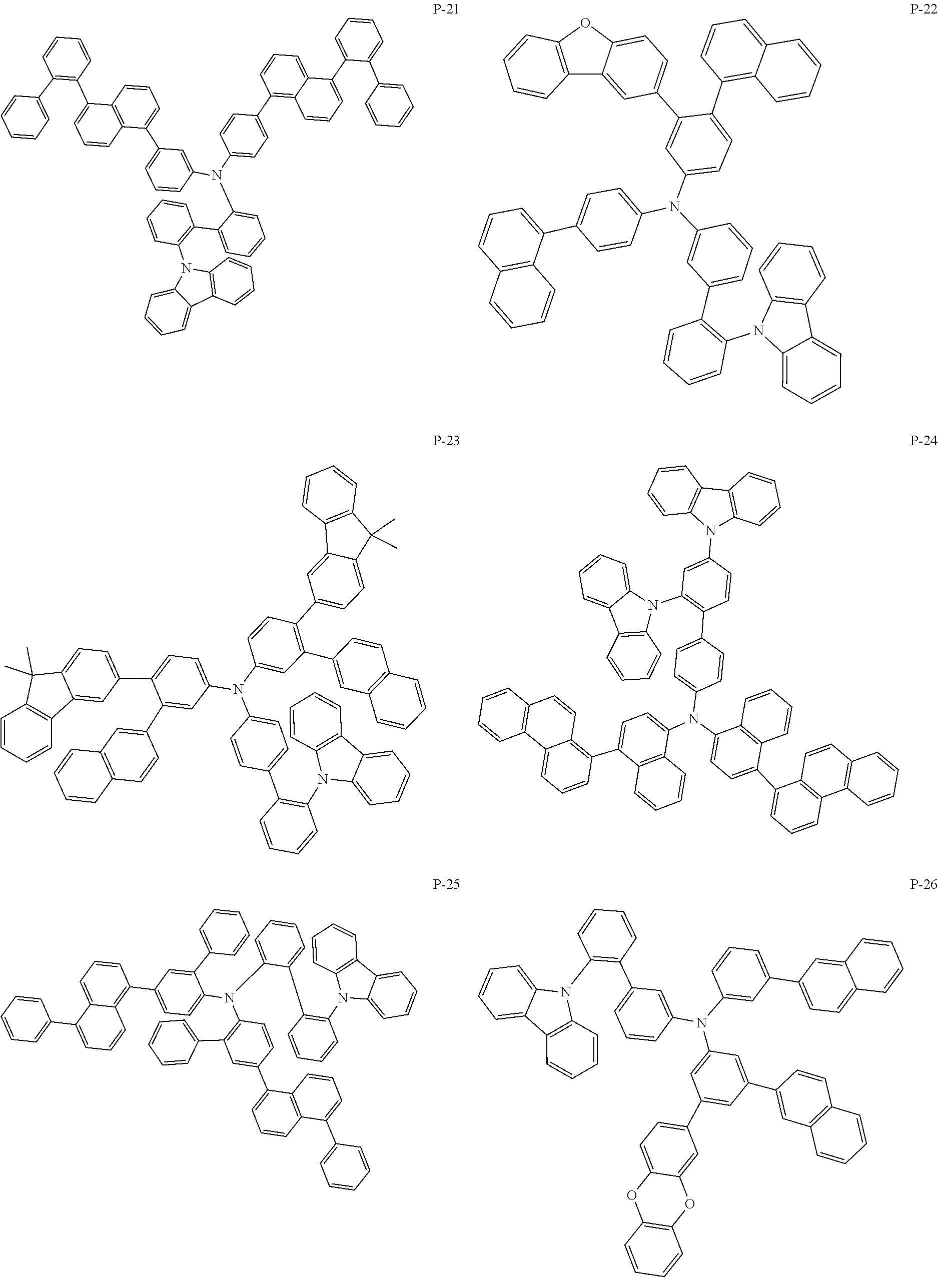

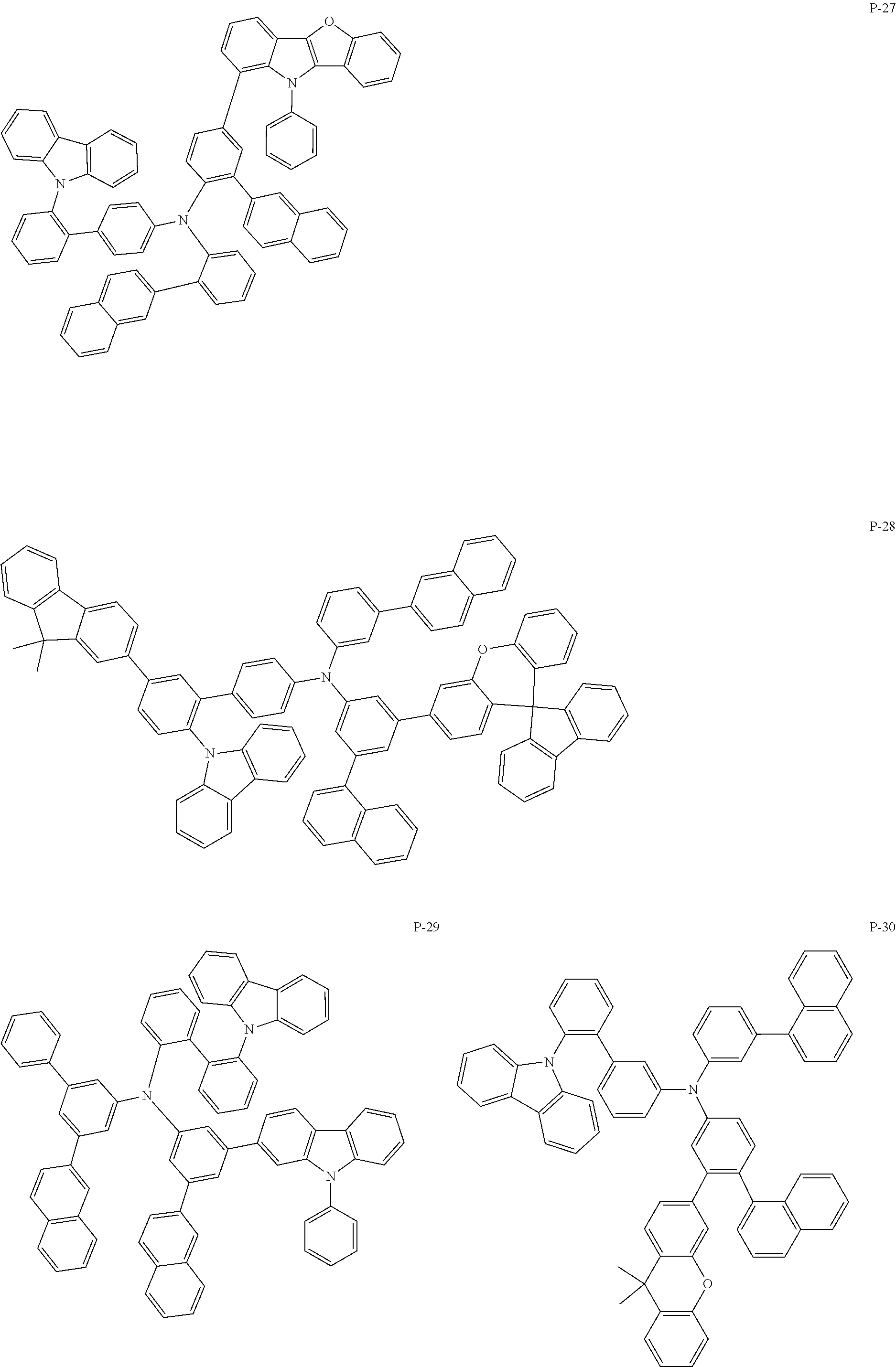

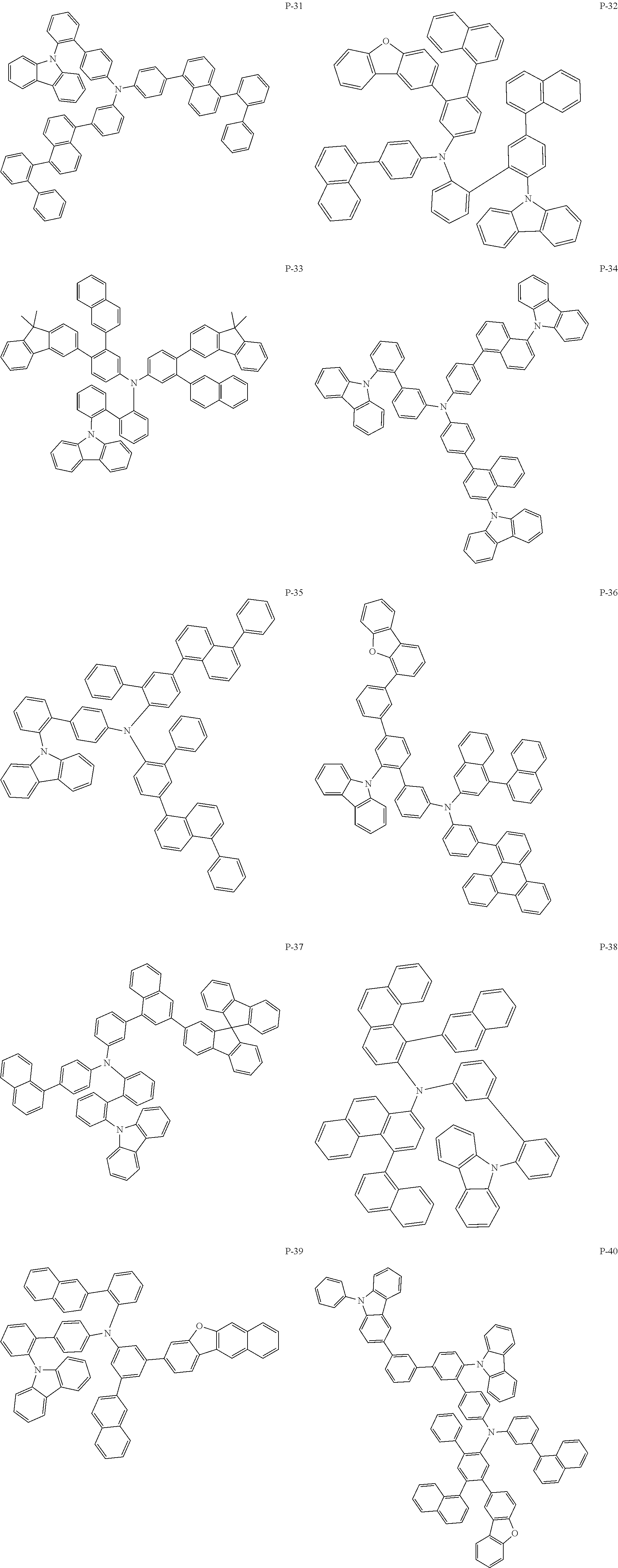

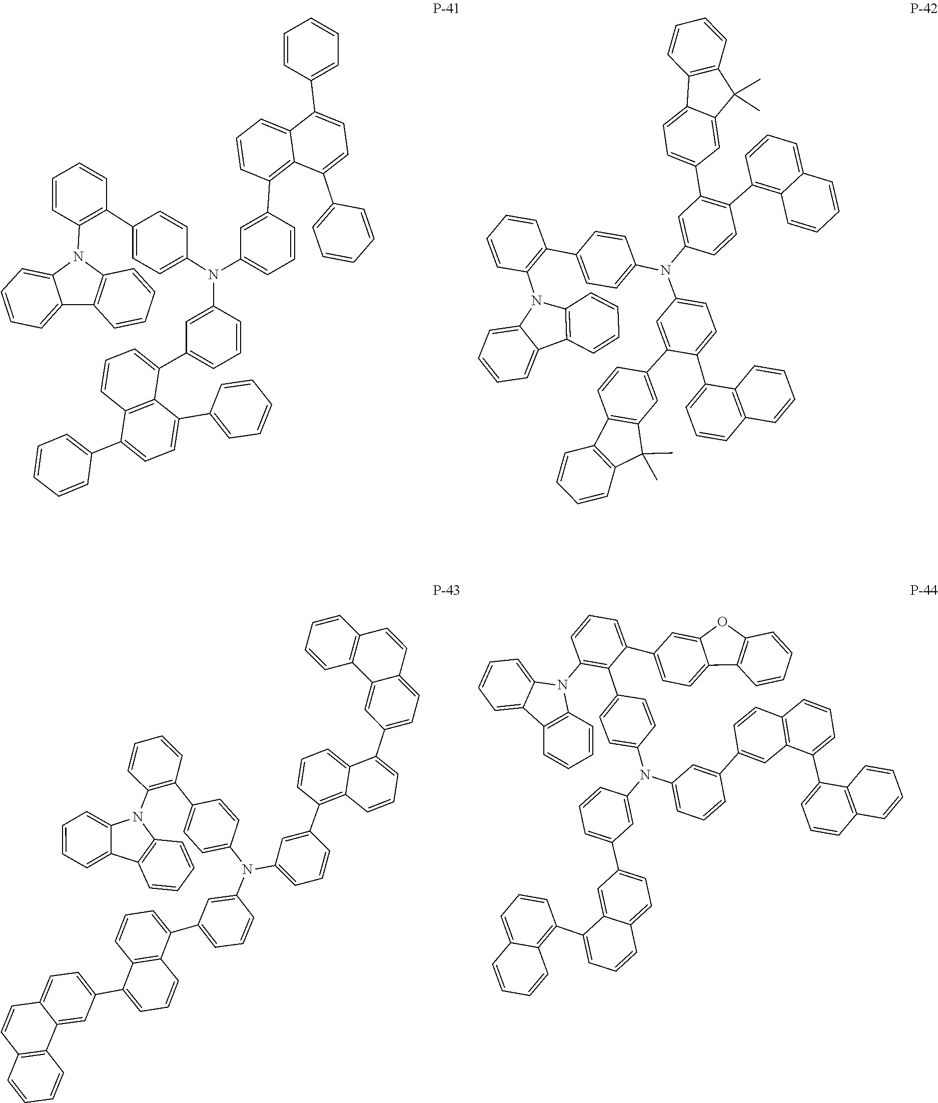

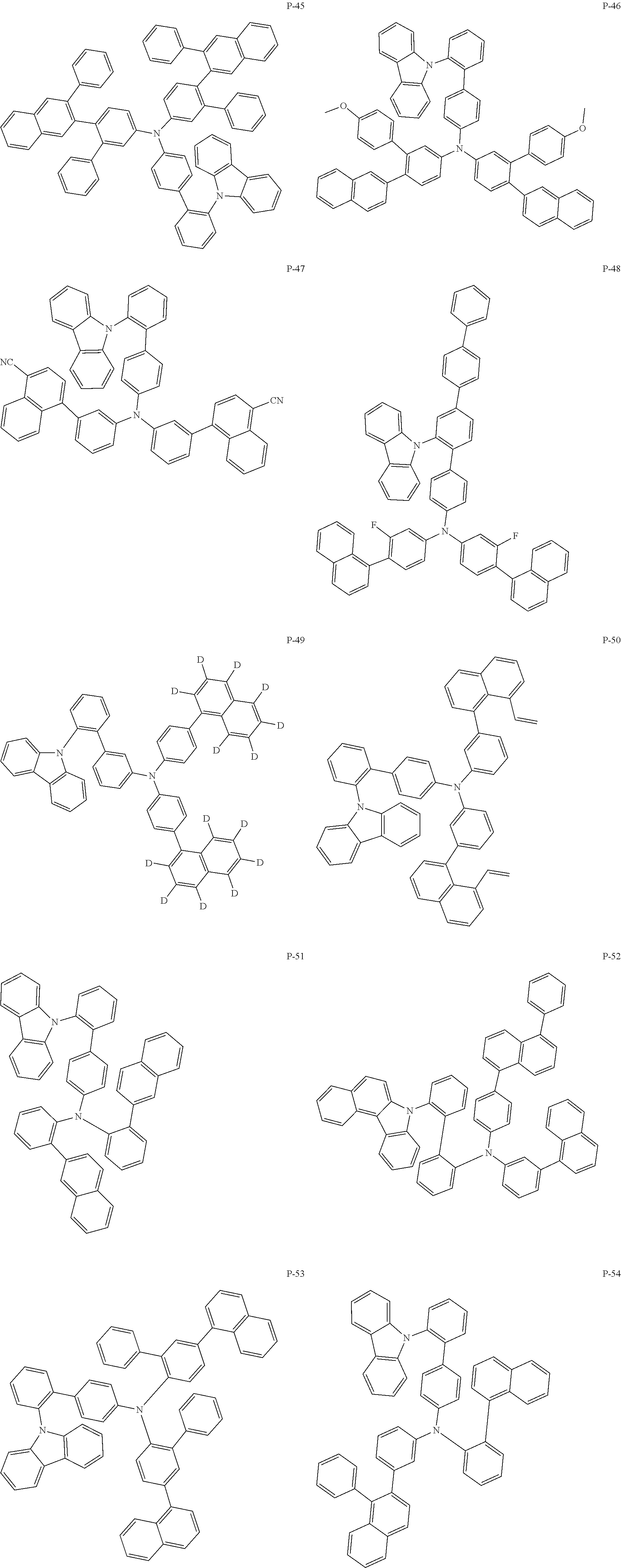

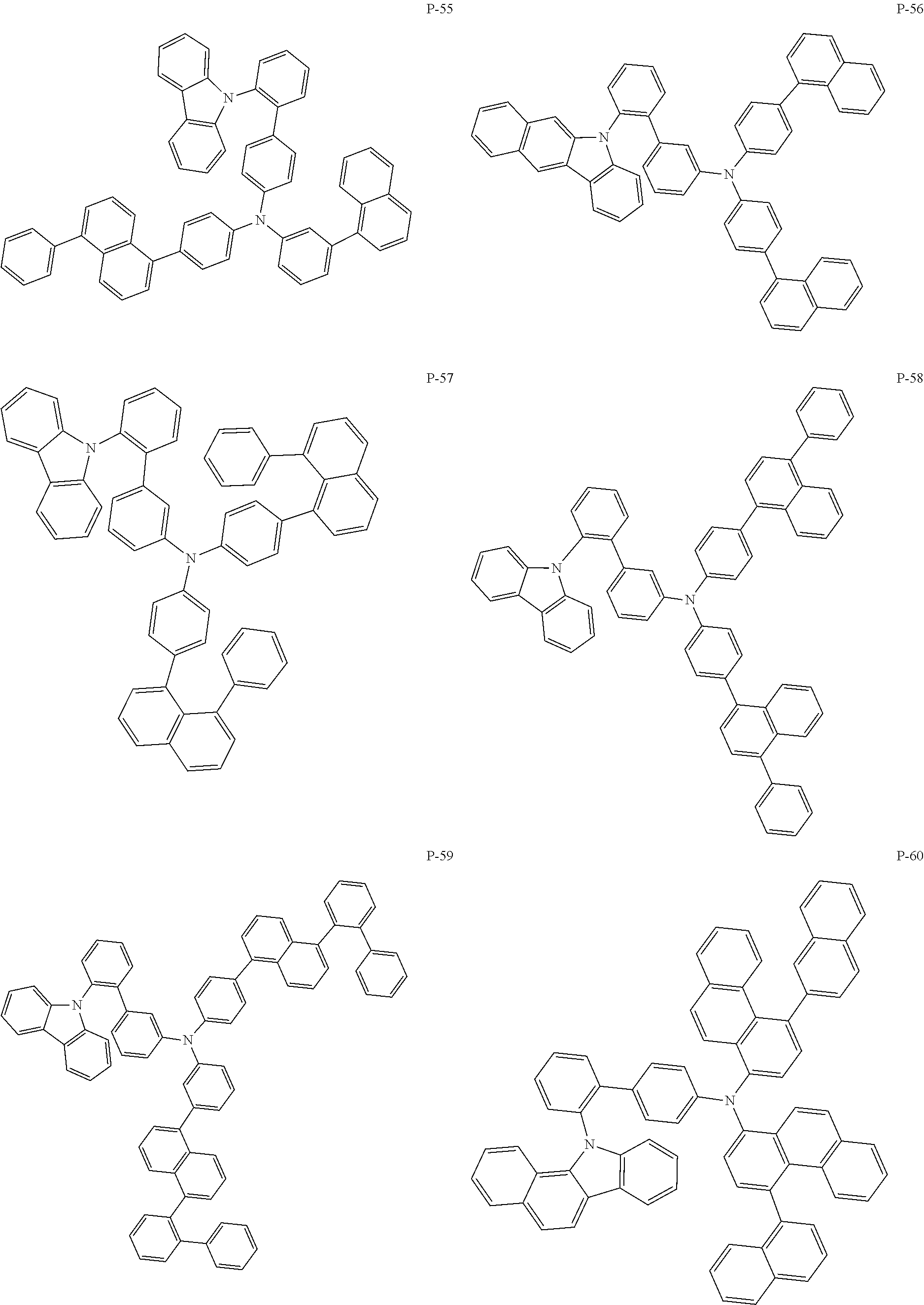

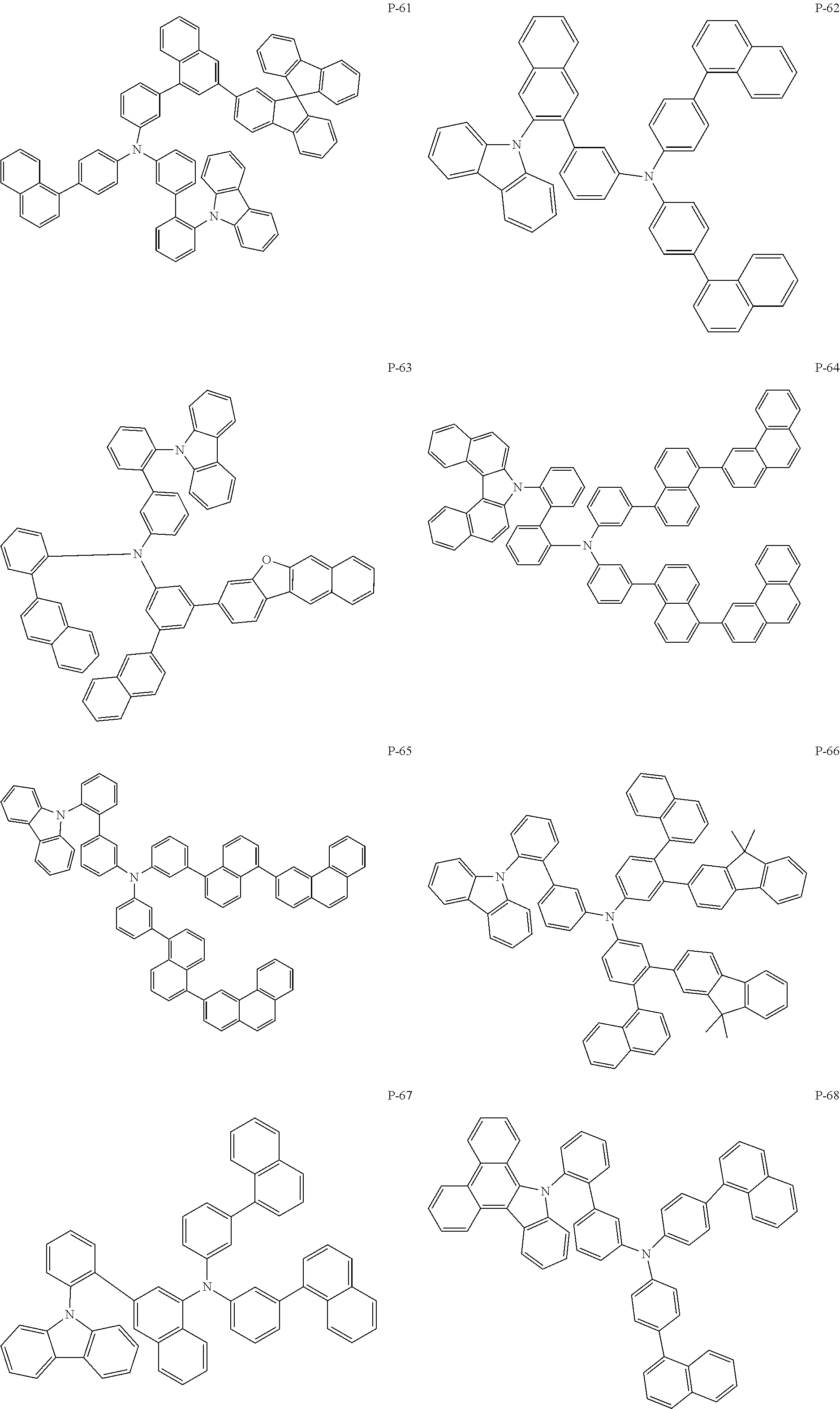

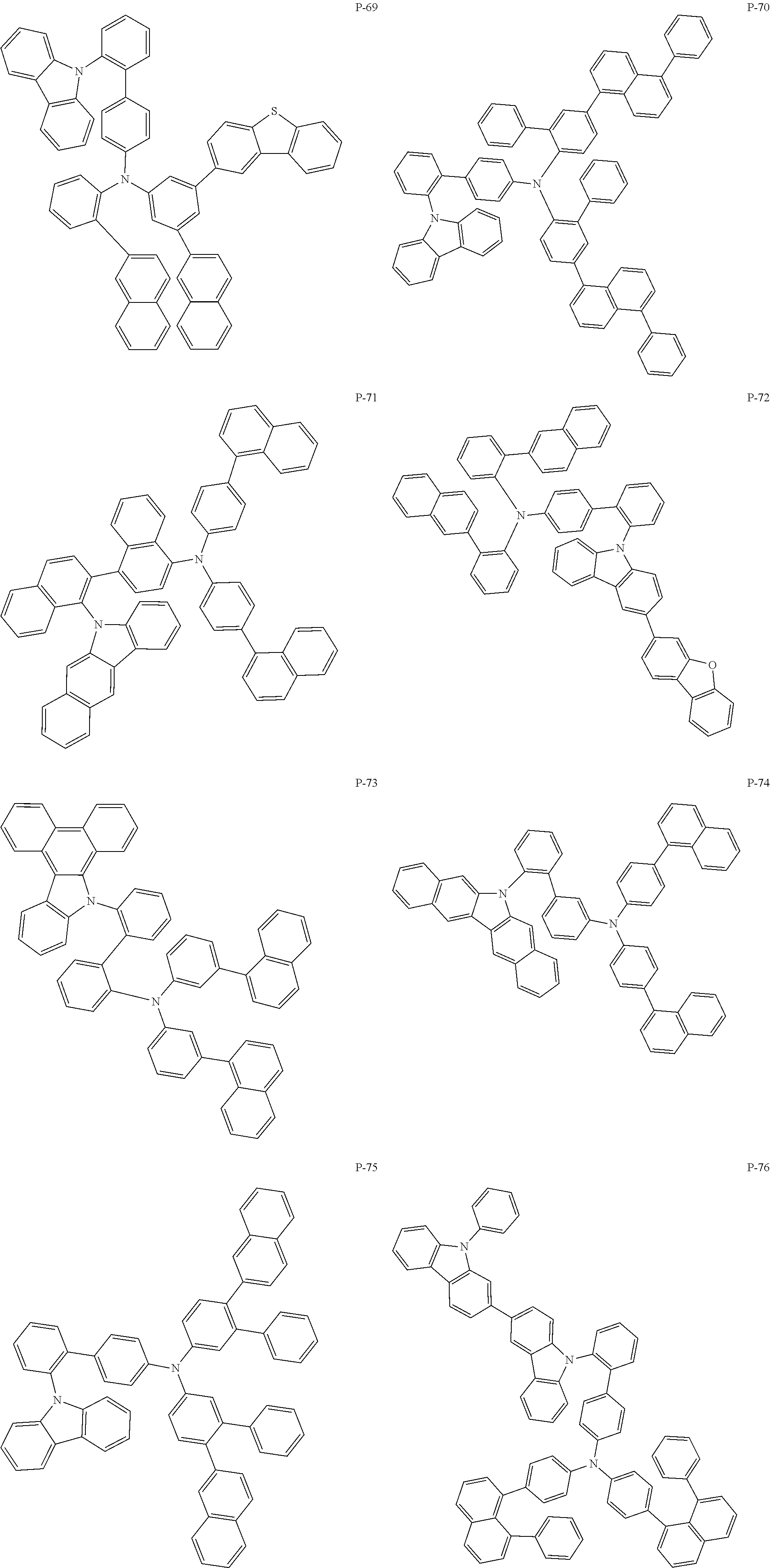

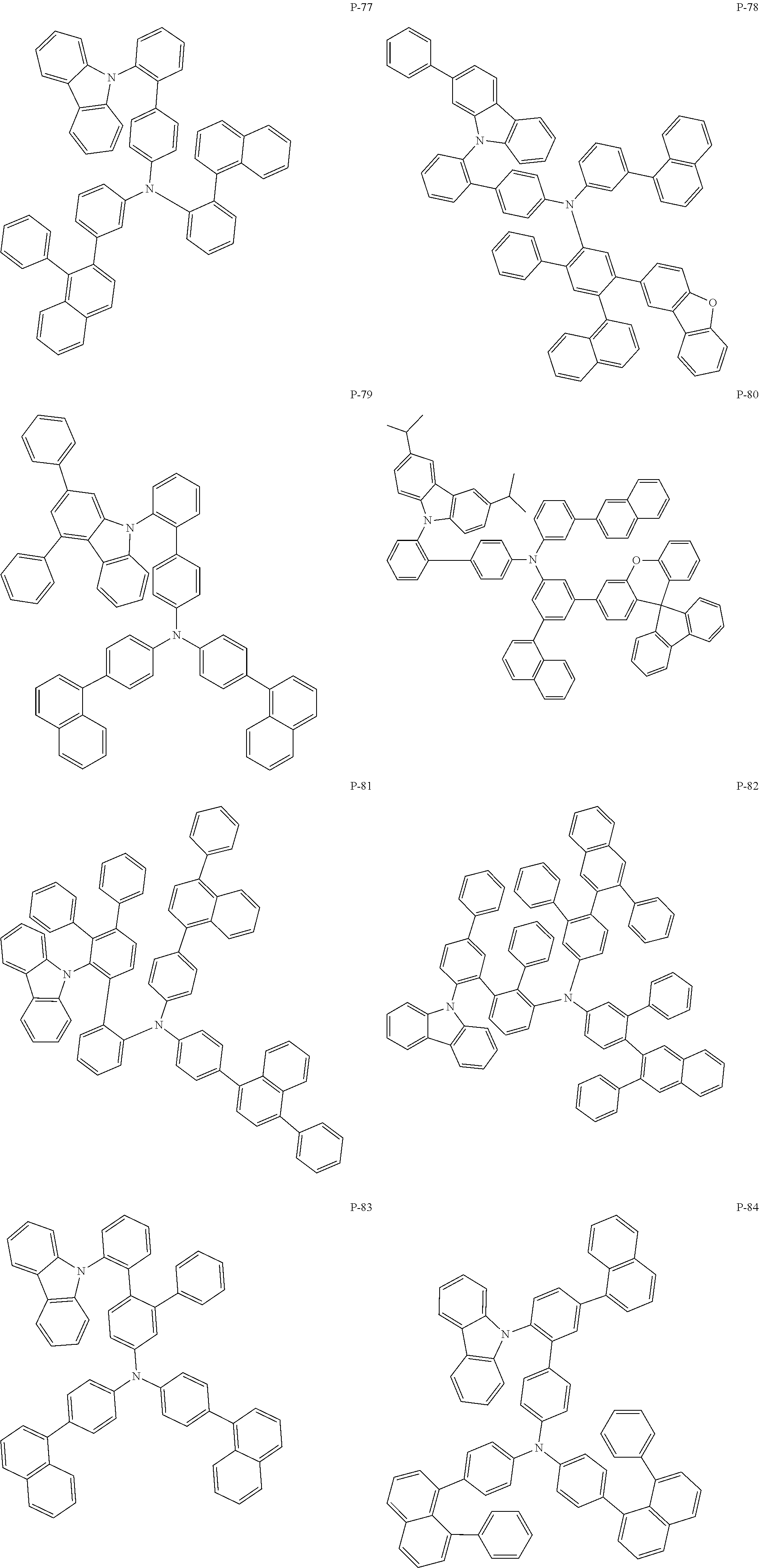

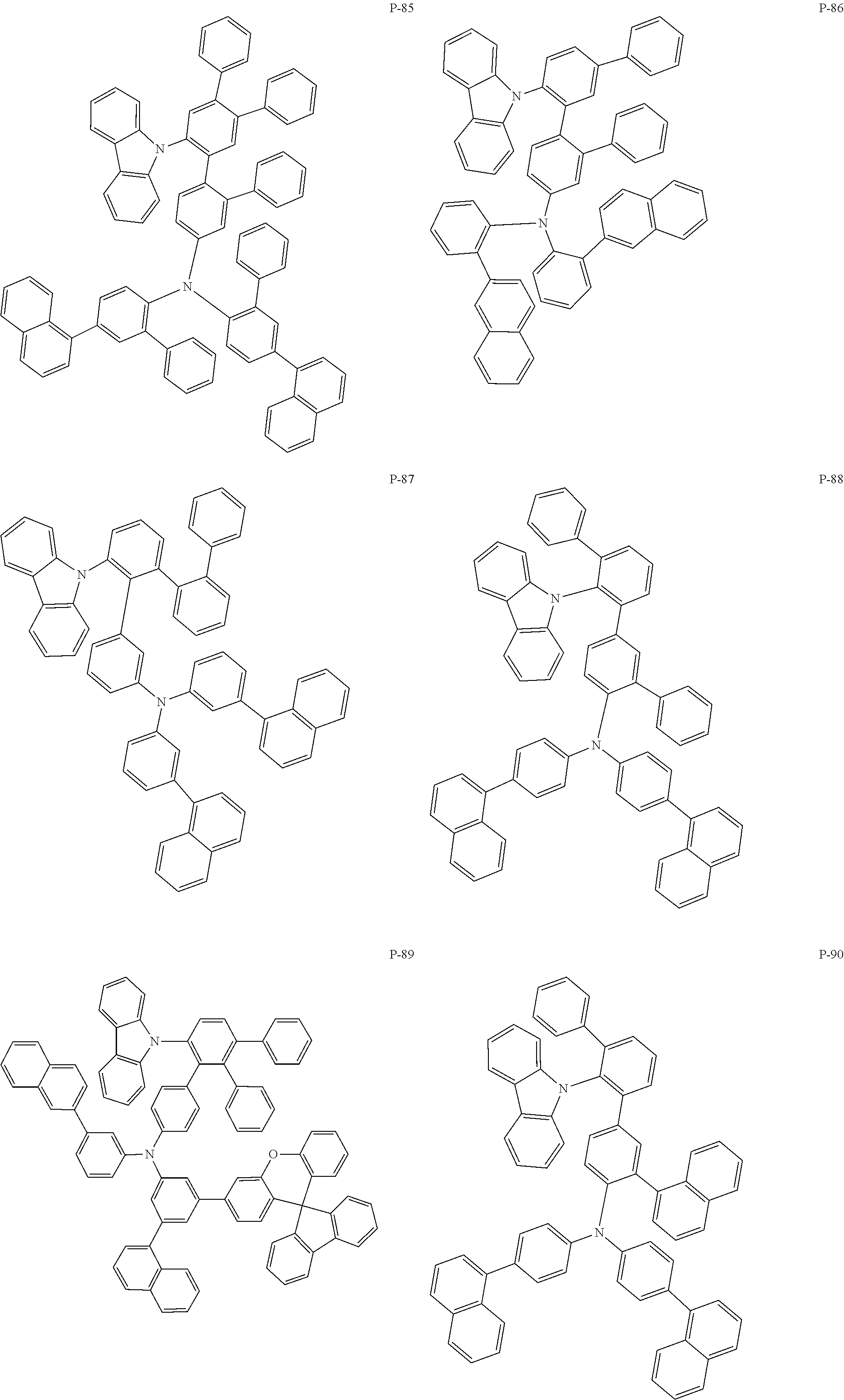

Also, the present invention provides an organic electronic element comprising a first electrode, a second electrode, and an organic material layer formed between the first electrode and the second electrode, wherein the organic material layer comprises a hole transport layer; an emitting layer; and an emitting auxiliary layer formed between the hole transport layer and the emitting layer; wherein the emitting auxiliary layer comprises the compound represented by Formula 1.

Also, the emitting layer is represented by any one of Formulas 2 to 4.

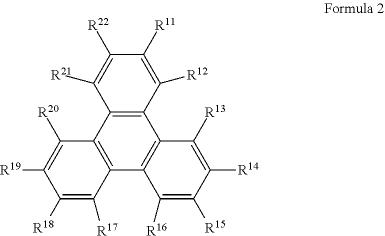

In Formula 2, each symbol may be defined as follows.

1) R¹¹, R¹², R¹³, R¹⁴, R¹⁵, R¹⁶, R¹⁷, R¹⁸, R¹⁹, R²⁰, R²¹ and R²² are each independently selected from the group consisting of hydrogen; deuterium; a C₆-C₆₀ aryl group; a C₂-C₆₀ heterocyclic group including at least one heteroatom of O, N, S, Si or P; a C₁-C₅₀ alkyl group; a C₁-C₅₀ alkoxy group; a C₂-C₂₀ alkenyl group; and -L′-NR^{a}R^{b}; or alternatively, adjacent groups may be bonded to each other to form a ring.

Wherein in case R¹¹, R¹², R¹³, R¹⁴, R¹⁵, R¹⁶, R¹⁷, R¹⁸, R¹⁹, R²⁰, R²¹ and R²² are an alkyl group, it may be preferably a C₁-C₃₀ alkyl group, and more preferably a C₁-C₂₄ alkyl group,

wherein in case R¹¹, R¹², R¹³, R¹⁴, R¹⁵, R¹⁶, R¹⁷, R¹⁸, R¹⁹, R²⁰, R²¹ and R²² are an alkoxyl group, it may be preferably an C₁˜C₃₀ alkoxyl group, and more preferably an C₁˜C₂₄ alkoxyl group,

wherein in case R¹¹, R¹², R¹³, R¹⁴, R¹⁵, R¹⁶, R¹⁷, R¹⁸, R¹⁹, R²⁰, R²¹ and R²² are an aryl group, it may be preferably a C₆-C₃₀ aryl group, and more preferably a C₆-C₂₅ aryl group, for example, it may be phenylene, biphenyl, naphthalene, terphenyl, etc,

wherein in case R¹¹, R¹², R¹³, R¹⁴, R¹⁵, R¹⁶, R¹⁷, R¹⁸, R¹⁹, R²⁰, R²¹ and R²² are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

2) L′ is selected from the group consisting of a single bond; a C₆-C₆₀ arylene group; fluorenylene group; a C₂-C₆₀ heterocyclic group including at least one heteroatom of O, N, S, Si or P; a C₃-C₆₀ aliphatic ring;

wherein in case L′ is an arylene group, it may be preferably a C₆˜C₃₀ arylene group, more preferably a C₆˜C₂₄ arylene group, for example, phenylene, biphenyl, naphthalene, terphenyl, etc.,

wherein in case L′ is a heterocyclic group, it may be preferably a C₂-C₃₀ heterocyclic group, and more preferably a C₂-C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

where L′ is an aliphatic ring group, it may be preferably a C₃-C₃₀ aliphatic group, more preferably a C₃-C₂₄ aliphatic ring group.

3) wherein R^{a} and R^{b} are each independently selected from the group consisting of a C₆˜C₆₀ aryl group; fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring;

wherein in case R^{a} and R^{b} are an aryl group, it may be preferably a C₆-C₃₀ aryl group, more preferably a C₆-C₂₅ aryl group, for example, phenyl, biphenyl, naphthyl, phenanthrene, terphenyl, etc.,

wherein in case R^{a} and R^{b} are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

wherein in case R^{a} and R^{b} are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring,

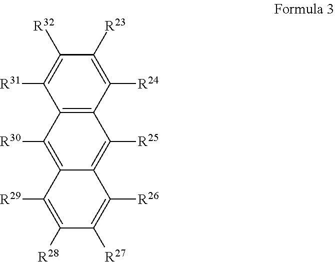

In Formula 3, each symbol may be defined as follows.

1) R²³, R²⁴, R²⁵, R²⁶, R²⁷, R²⁸, R²⁹, R³⁰, R³¹ and R³² are each independently selected from the group consisting of hydrogen; deuterium; halogen; a C₆-C₆₀ aryl group; fluorenyl group; a C₂-C₆₀ heterocyclic group including at least one heteroatom of O, N, S, Si or a fused ring group of a C₃-C₆₀ aliphatic ring and a C₆-C₆₀ aromatic ring; a C₁-C₅₀ alkyl group; C₂-C₂₀ alkenyl group; C₂-C₂₀ alkynyl group; a C₁-C₃₀ alkoxyl group; a C₆-C₃₀ aryloxy group; and -L′-NR^{a}R^{b}, or alternatively, adjacent groups may be bonded to each other to form a ring.

wherein in case R²³, R²⁴, R²⁵, R²⁶, R²⁷, R²⁸, R²⁹, R³⁰, R³¹ and R³² are an alkyl group, it may be preferably a C₁-C₃₀ alkyl group, and more preferably a C₁-C₂₄ alkyl group,

wherein in case R²³, R²⁴, R²⁵, R²⁶, R²⁷, R²⁸, R²⁹, R³⁰, R³¹ and R³² are an alkoxyl group, it may be preferably an C₁˜C₂₄ alkoxyl group,

wherein in case R²³, R²⁴, R²⁵, R²⁶, R²⁷, R²⁸, R²⁹, R³⁰, R³¹ and R³² are an aryloxy group, it may be preferably an C₆˜C₂₄ aryloxy group,

wherein in case R²³, R²⁴, R²⁵, R²⁶, R²⁷, R²⁸, R²⁹, R³⁰, R³¹ and R³² are an aryl group, it may be preferably a C₆-C₃₀ aryl group, and more preferably a C₆-C₂₄ aryl group, for example, it may be phenylene, biphenyl, naphthalene, terphenyl, etc,

wherein in case R²³, R²⁴, R²⁵, R²⁶, R²⁷, R²⁸, R²⁹, R³⁰, R³¹ and R³² are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.,

wherein in case R²³, R²⁴, R²⁵, R²⁶, R²⁷, R²⁸, R²⁹, R³⁰, R³¹ and R³² are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

2) wherein L′, R^{a} and R^{b} are the same as defined in Formula 2.

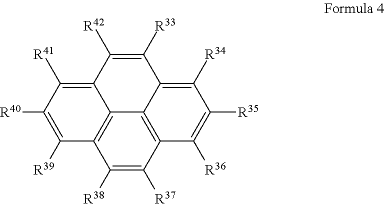

wherein , R³³, R³⁴, R³⁵, R³⁶, R³⁷, R³⁸, R³⁹, R⁴⁰, R⁴¹ and R⁴² are the same as the definition of R²³ in Formula 3.

Also, Formula 2 is represented by any one of the following compounds 8-1 to 8-18.

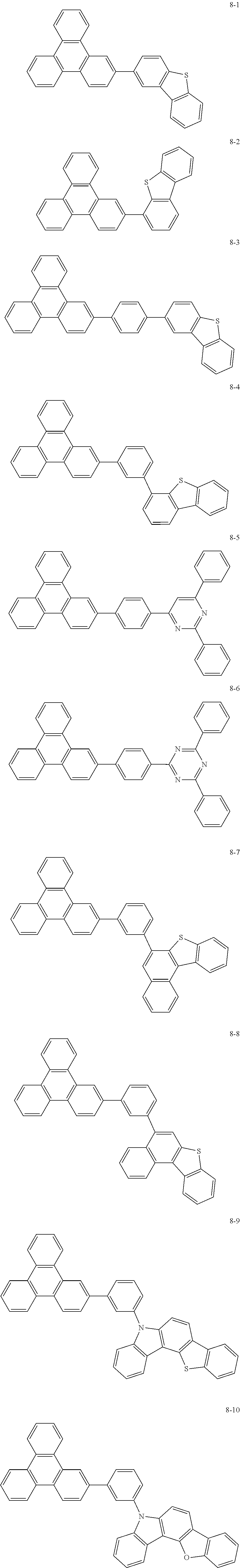

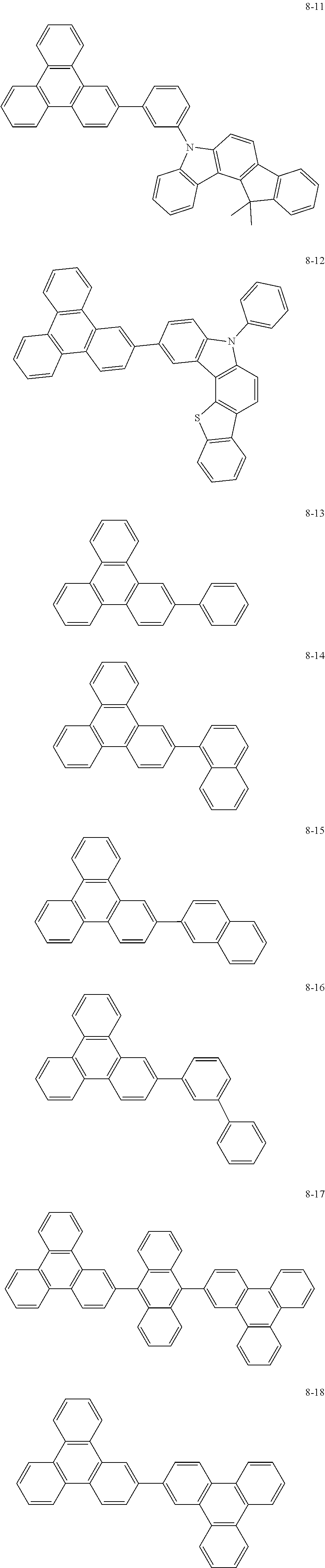

Also, Formula 3 is represented by any one of the following compounds 9-1 to 9-32.

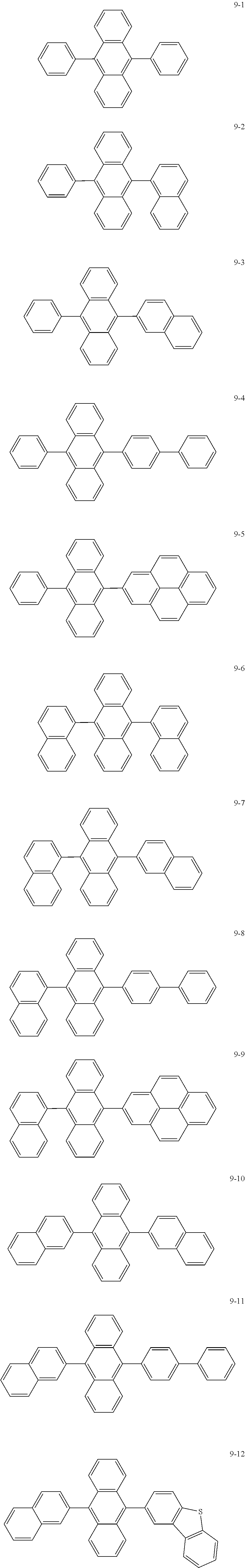

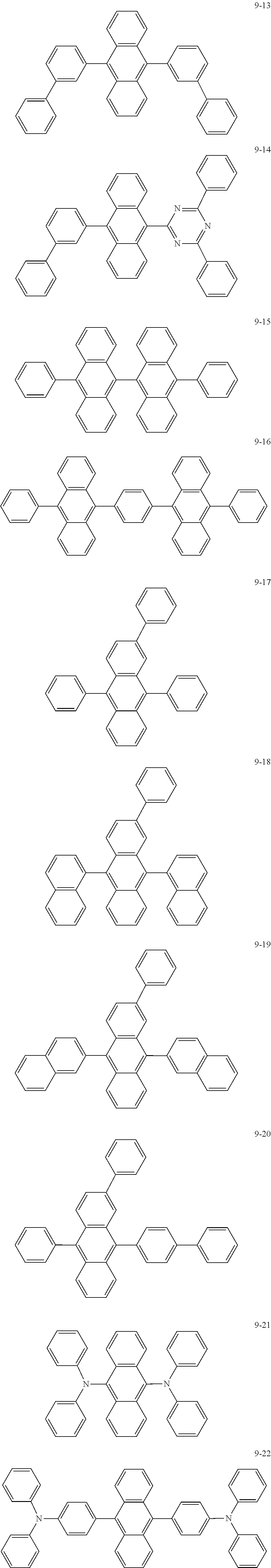

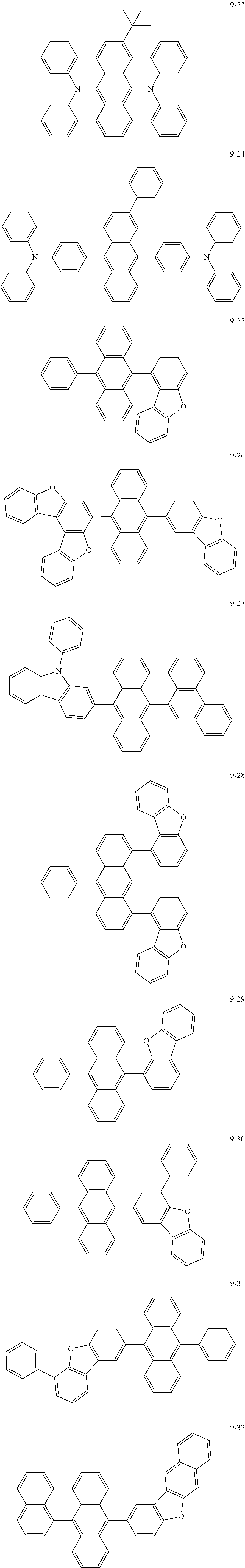

Also, Formula 4 is represented by any one of the following compounds 10-1 to 10-20.

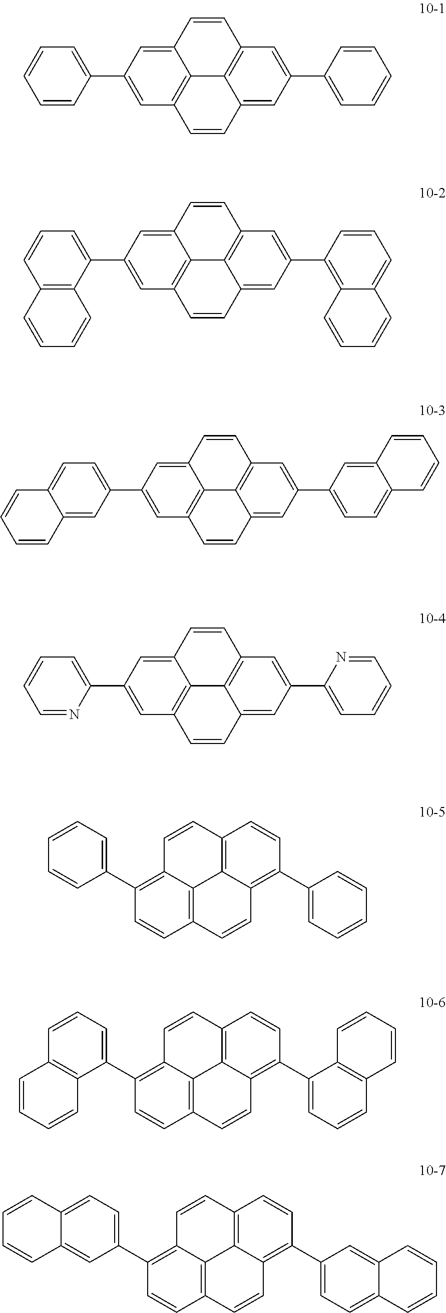

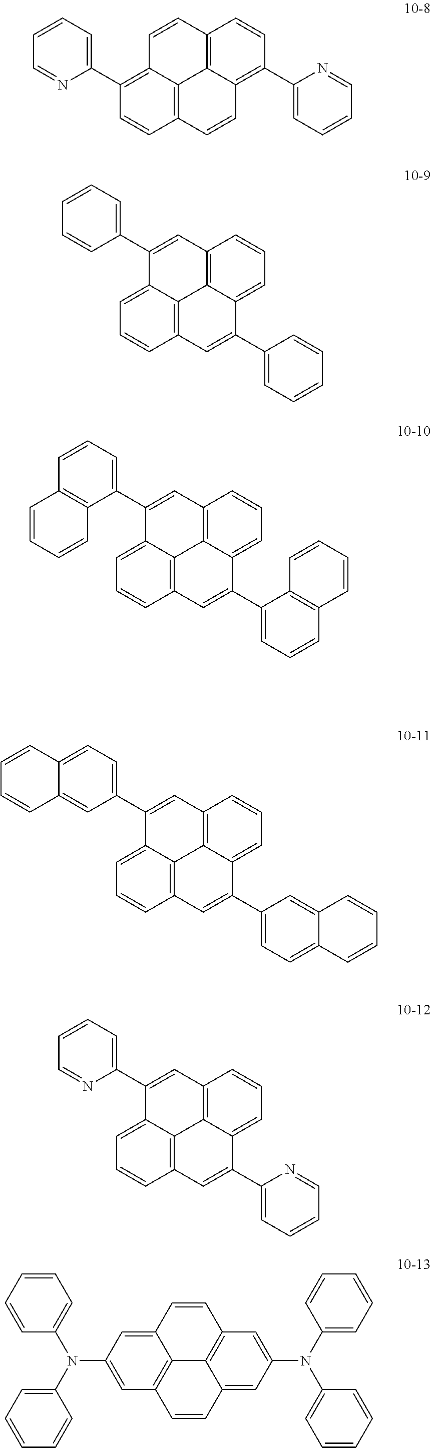

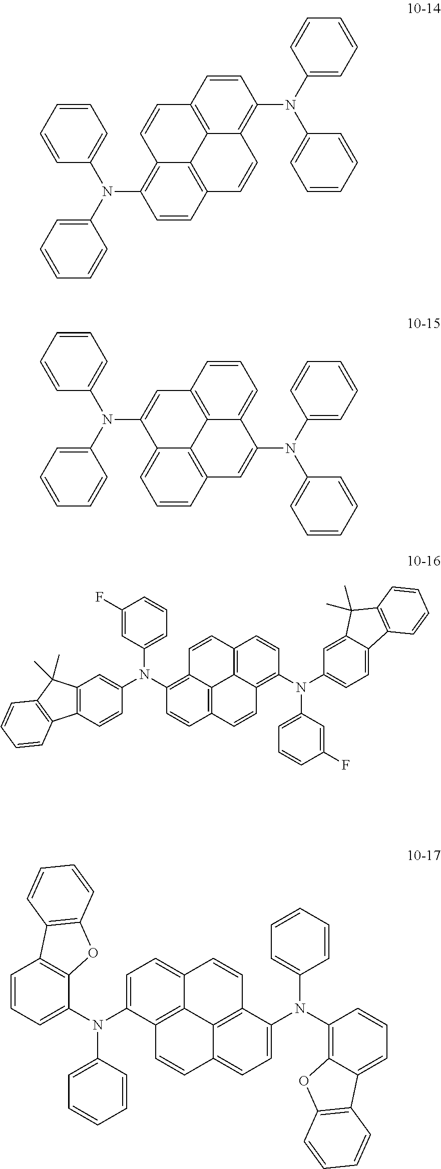 [SMILES: c1ccc(N(c2ccccc2)c2ccc3ccc4c(N(c5ccccc5)c5ccccc5)ccc5ccc2c3c54)cc1 ; c1ccc(N(c2ccccc2)c2cc3cccc4c(N(c5ccccc5)c5ccccc5)cc5cccc2c5c34)cc1 ; CC1(C)c2ccccc2-c2ccc(N(c3cccc(F)c3)c3ccc4ccc5c(N(c6cccc(F)c6)c6ccc7c(c6)C(C)(C)c6ccccc6-7)ccc6ccc3c4c65)cc21 ; c1ccc(N(c2ccc3ccc4c(N(c5ccccc5)c5cccc6c5oc5ccccc56)ccc5ccc2c3c54)c2cccc3c2oc2ccccc23)cc1]

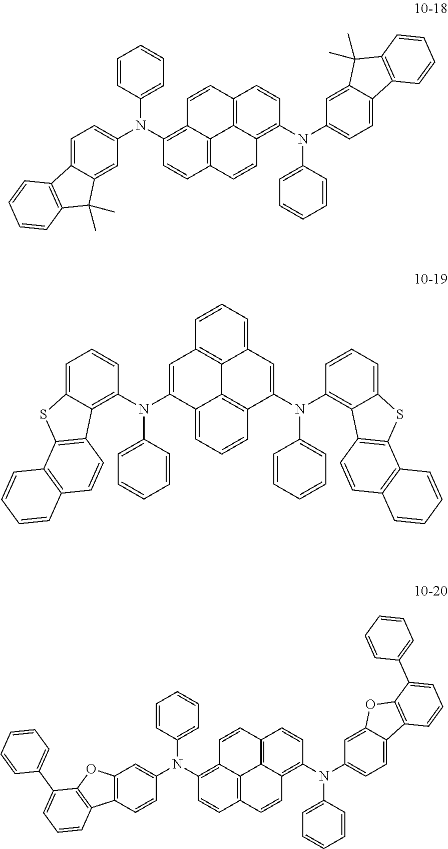 [SMILES: CC1(C)c2ccccc2-c2ccc(N(c3ccccc3)c3ccc4ccc5c(N(c6ccccc6)c6ccc7c(c6)C(C)(C)c6ccccc6-7)ccc6ccc3c4c65)cc21 ; c1ccc(N(c2cc3cccc4cc(N(c5ccccc5)c5cccc6sc7c8ccccc8ccc7c56)c5cccc2c5c34)c2cccc3sc4c5ccccc5ccc4c23)cc1 ; c1ccc(-c2cccc3c2oc2cc(N(c4ccccc4)c4ccc5ccc6c(N(c7ccccc7)c7ccc8c(c7)oc7c(-c9ccccc9)cccc78)ccc7ccc4c5c76)ccc23)cc1]

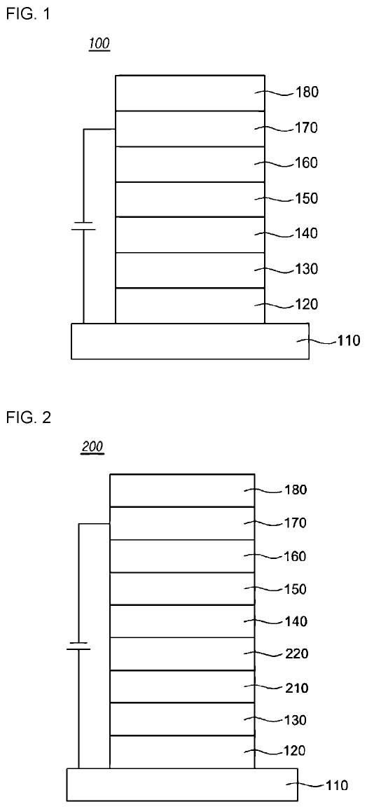

Referring to FIG. 1, the organic electronic element (100) according to the present invention comprises a first electrode (110), a second electrode (170), and an organic material layer comprising a single compound or 2 or more compounds represented by Formula 1 between the first electrode (110) and the second electrode (170). Wherein, the first electrode (110) may be an anode, the second electrode (170) may be a cathode. In the case of an invert type, the first electrode may be a cathode and the second electrode may be an anode.

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2)

Also, the organic electronic element according to an embodiment of the present invention may further include a protective layer or a light efficiency enhancing layer (180). The light efficiency enhancing layer may be formed on one of both surfaces of the first electrode, the surface not in contact with the organic material layer or on one of both surfaces of the second electrode, the surface not in contact with the organic material layer. The compound according to an embodiment of the present invention applied to the organic material layer may be used as a material for the hole injection layer (120), the hole transport layer (130), the emitting-auxiliary layer (220), electron transport auxiliary layer, the electron transport layer (150), and an electron injection layer (160), a host or dopant of the emitting layer (140) or the light efficiency enhancing layer. Preferably, for example, the compound according to Formula 1 of the present invention may be used as a material for an emitting auxiliary layer and the compound according to any one of Formulas 2 to 4 may be used as a material for the emitting layer.

The organic material layer may comprise 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials(mobility, interfacial characteristics, etc.) of each organic material layer is achieved, a long lifespan and high efficiency can be achieved at the same time.

The organic electroluminescent device according to an embodiment of the present invention may be manufactured using a PVD (physical vapor deposition) method. For example, depositing a metal or a metal oxide having conductivity or an alloy thereof on a substrate to form an anode, and after forming an organic material layer including the hole injection layer (120), the hole transport layer (130), the emitting layer (140), the electron transport layer (150) and the electron injection layer (160) thereon, it can be prepared by depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process, and the organic material layer provides an organic electronic element comprising the compound as an electron transport material.

As another specific example, the compound of the same or different types of the compound represented by Formula 1 is mixed and used in the organic material layer.

Also, the present invention provides an electronic device comprising a display device including the organic electric element; and a control unit for driving the display device;

In another aspect, the organic electric element is at least one of an organic electroluminescent device, an organic solar cell, an organic photo conductor, an organic transistor, and a device for monochromatic or white lighting. At this time, the electronic device may be a current or future wired/wireless communication terminal, and covers all kinds of electronic devices including mobile communication terminals such as mobile phones, a personal digital assistant(PDA), an electronic dictionary, a point-to-multipoint(PMP), a remote controller, a navigation unit, a game player, various kinds of TVs, and various kinds of computers.

Hereinafter, a synthesis example of the compound represented by Formula 1 of the present invention and a manufacturing example of an organic electric element of the present invention will be described in detail with reference to Examples, but the present invention is not limited to the following Examples.

### Synthesis Example 1

The compound (Final Product) represented by Formula 1 according to the present invention is synthesized as shown in Reaction Scheme 1, but is not limited thereto. Hal is I, Br or Cl.

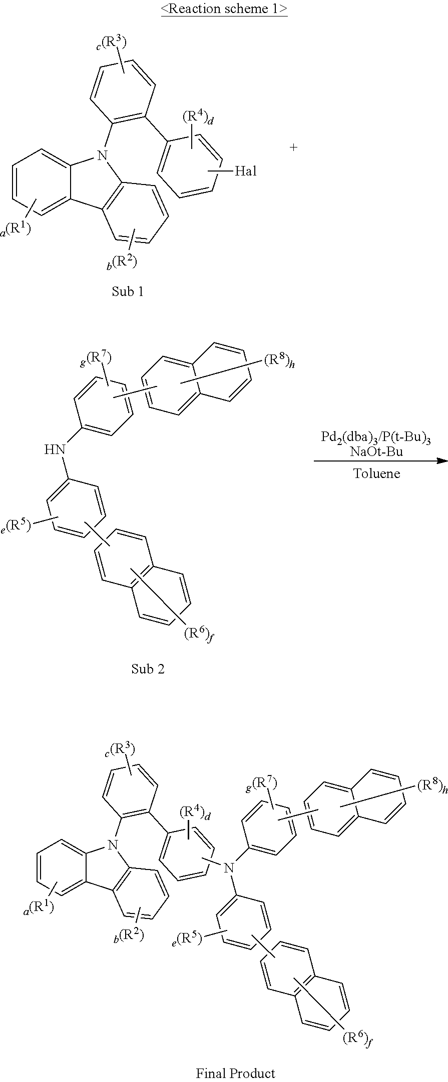

### I. Synthesis of Sub 1

Sub 1 of Reaction Scheme 1 is synthesized by the reaction pathway of Reaction Scheme 2, but is not limited thereto. Hal is I, Br or Cl.

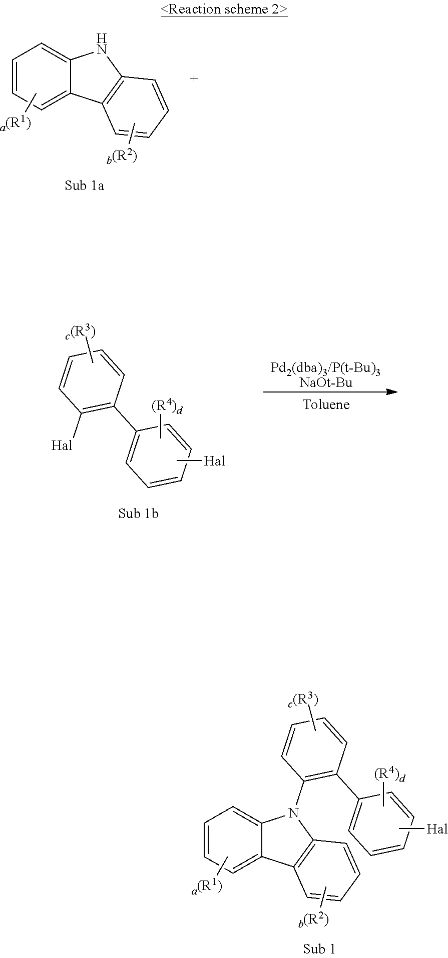

### 1. Synthesis Example of Sub 1-2

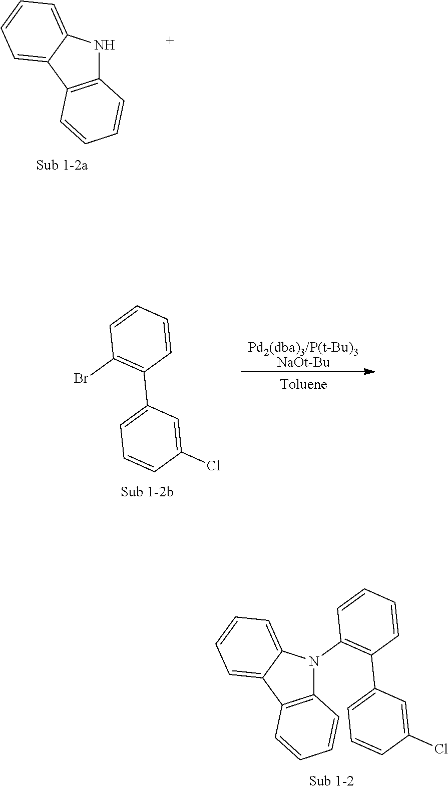 [SMILES: c1ccc2c(c1)[nH]c1ccccc12 ; Clc1cccc(-c2ccccc2Br)c1 ; "Pd2(dba)3/P(t-Bu)3NaOt-Bu" ; "Toluene" ; Clc1cccc(-c2ccccc2-n2c3ccccc3c3ccccc32)c1]

After adding Sub 1-2a (50.0 g, 299.0 mmol), Sub 1-2b (160.0 g, 598.1 mmol), Pd₂(dba)₃ (8.2 g, 9.0 mmol), P(t-Bu)₃ (3.6 g, 17.9 mmol), NaOt-Bu (57.5 g, 598.1 mmol), toluene (1495 mL) to a round bottom flask, the reaction proceeds at 80° C. After the reaction was completed, extraction was performed with CH2012 and water, and the organic layer was dried with MgSO₄, concentrated, and the resulting organic material was recrystallized using a silicagel column to obtain 82.1 g of the product. (Yield: 77.6%)

### 2. Synthesis Example of Sub 1-7

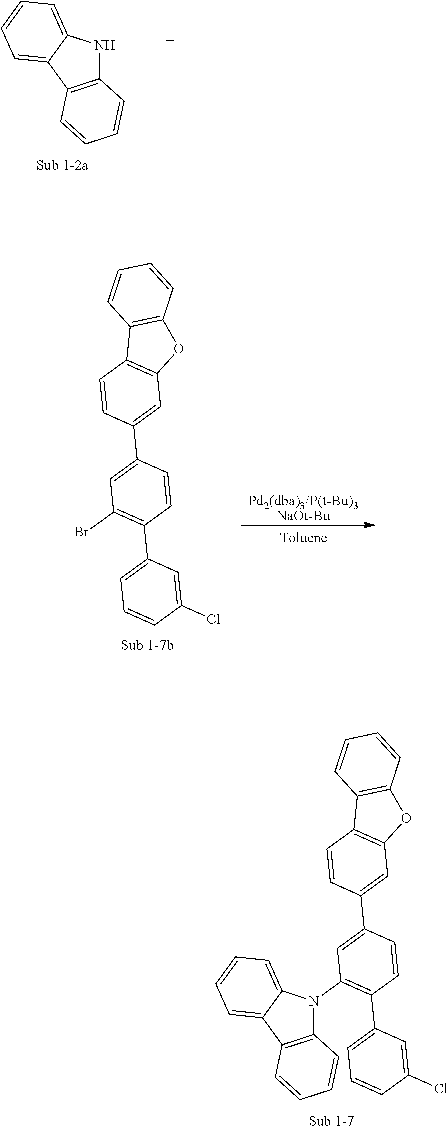 [SMILES: c1ccc2c(c1)[nH]c1ccccc12 ; Clc1cccc(-c2ccc(-c3ccc4c(c3)oc3ccccc34)cc2Br)c1 ; "Pd2(dba)3/P(t-Bu)3NaOt-Bu" ; "Toluene" ; Clc1cccc(-c2ccc(-c3ccc4c(c3)oc3ccccc34)cc2-n2c3ccccc3c3ccccc32)c1]

Sub 1-2a (50.0 g, 299.0 mmol), Sub 1-7b (259.4 g, 598.1 mmol), Pd₂(dba)₃ (8.2 g, 9.0 mmol), P(t-Bu)₃ (3.6 g, 17.9 mmol), NaOt-Bu (57.5 g, 598.1 mmol), Toluene (1495 mL) were added to a round bottom flask in the same manner as in Sub 1-2 to obtain 110.7 g of product. (Yield: 71.2%)

### 3. Synthesis Example of Sub 1-18

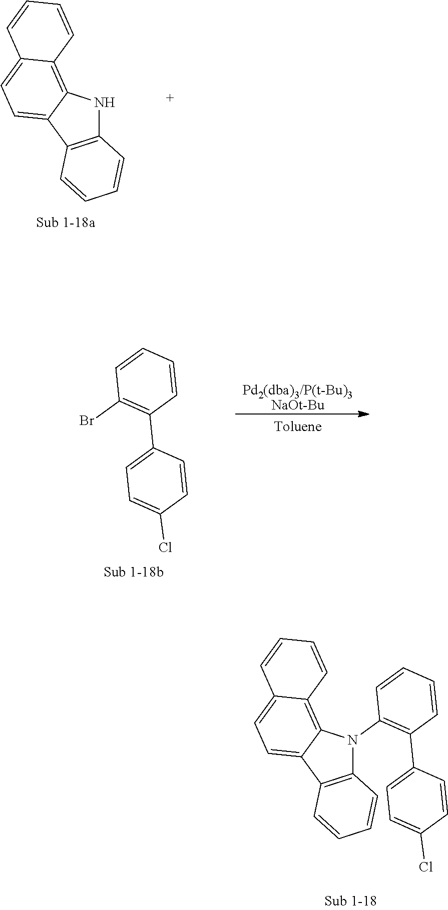 [SMILES: c1ccc2c(c1)ccc1c3ccccc3[nH]c21 ; Clc1ccc(-c2ccccc2Br)cc1 ; "Pd2(dba)3/P(t-Bu)3NaOt-Bu" ; "Toluene" ; Clc1ccc(-c2ccccc2-n2c3ccccc3c3ccc4ccccc4c32)cc1]

Sub 1-18a (50.0 g, 230.1 mmol), Sub 1-18b (123.1 g, 460.3 mmol), Pd₂(dba)₃ (6.3 g, 6.9 mmol), P(t-Bu)₃ (2.8 g, 13.8 mmol), NaOt-Bu (44.2 g, 460.3 mmol), toluene (1151 mL) were added to a round bottom flask in the same manner as in Sub 1-2 to obtain 69.3 g of product. (Yield: 74.6%)

### 4. Synthesis Example of Sub 1-23

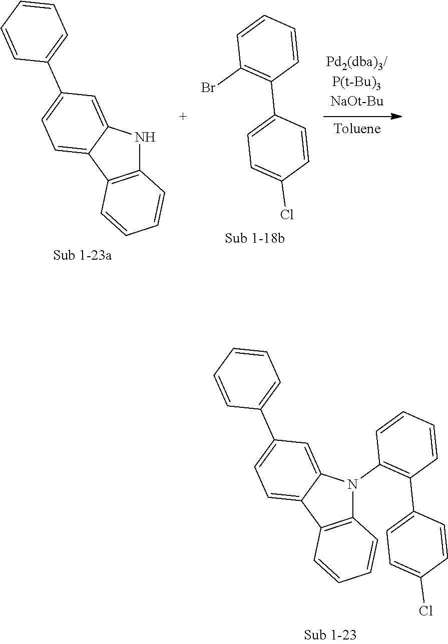 [SMILES: c1ccc(-c2ccc3c(c2)[nH]c2ccccc23)cc1 ; Clc1ccc(-c2ccccc2Br)cc1 ; Clc1ccc(-c2ccccc2-n2c3ccccc3c3ccc(-c4ccccc4)cc32)cc1]

Sub 1-23a (50.0 g, 205.5 mmol), Sub 1-18b (110.0 g, 411.0 mmol), Pd₂(dba)₃ (5.7 g, 6.2 mmol), P(t-Bu)₃ (2.5 g, 12.3 mmol), NaOt-Bu (39.5 g, 411.0 mmol), toluene (1027 mL) were added to a round bottom flask in the same manner as in Sub 1-2 to obtain 68.2 g of product. (Yield: 77.2%)

### 5. Synthesis Example of Sub 1-39

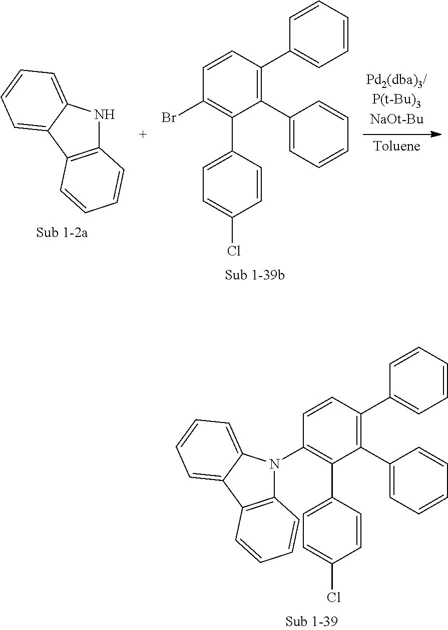 [SMILES: c1ccc2c(c1)[nH]c1ccccc12 ; Clc1ccc(-c2c(Br)ccc(-c3ccccc3)c2-c2ccccc2)cc1 ; Clc1ccc(-c2c(-n3c4ccccc4c4ccccc43)ccc(-c3ccccc3)c2-c2ccccc2)cc1]

Sub 1-2a (50.0 g, 299.0 mmol), Sub 1-39b (251.0 g, 598.1 mmol), Pd₂(dba)₃ (8.2 g, 9.0 mmol), P(t-Bu)₃ (3.6 g, 17.9 mmol), NaOt-Bu (57.5 g, 598.1 mmol), toluene (1495 mL) were added to a round bottom flask in the same manner as in Sub 1-2 to obtain 105.8 g of product. (Yield: 69.9%)

The compound belonging to Sub 1 may be the following compounds, but is not limited thereto, and Table 1 shows FD-MS (Field Desorption-Mass Spectrometry) values of compounds belonging to Sub 1.

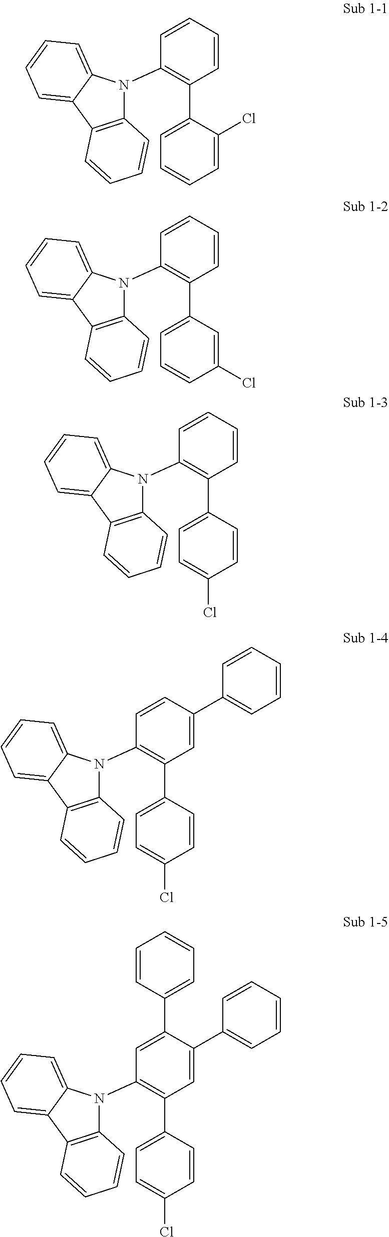

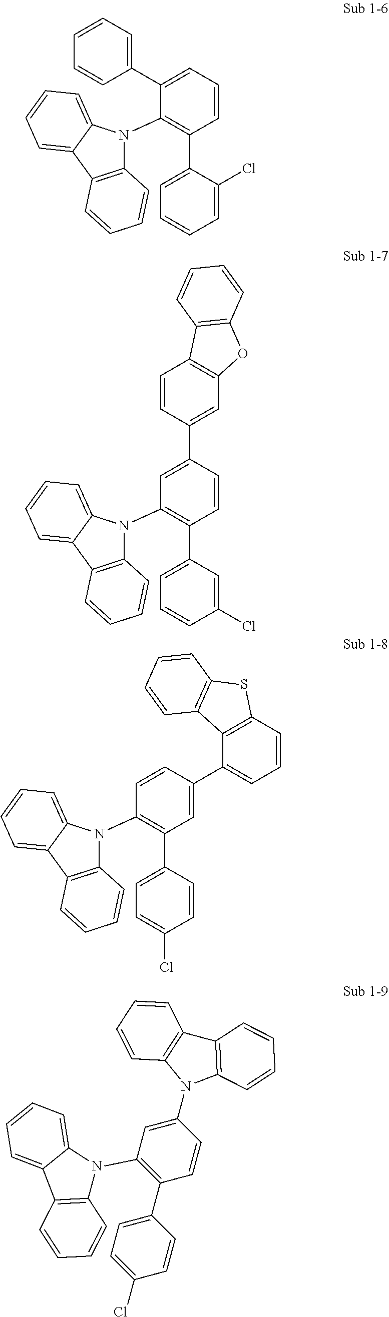

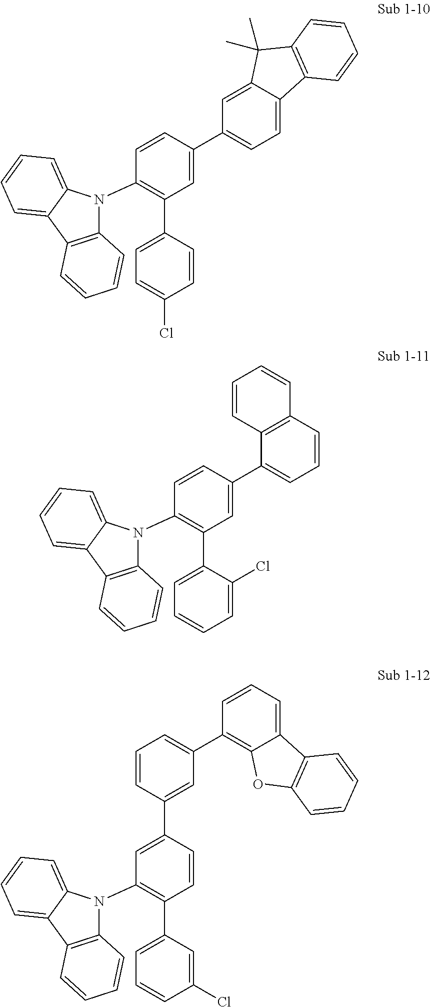 [SMILES: CC1(C)c2ccccc2-c2ccc(-c3ccc(-n4c5ccccc5c5ccccc54)c(-c4ccc(Cl)cc4)c3)cc21 ; Clc1ccccc1-c1cc(-c2cccc3ccccc23)ccc1-n1c2ccccc2c2ccccc21 ; Clc1cccc(-c2ccc(-c3cccc(-c4cccc5c4oc4ccccc45)c3)cc2-n2c3ccccc3c3ccccc32)c1]

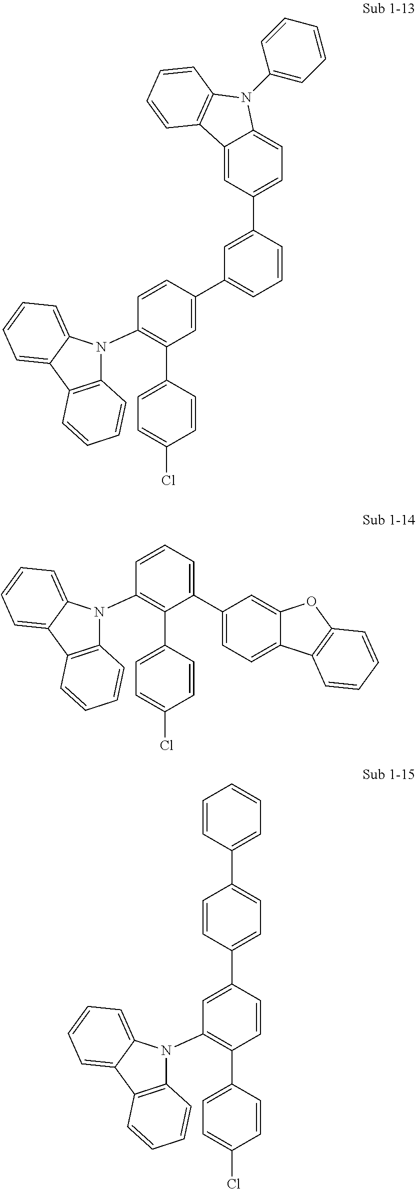 [SMILES: Clc1ccc(-c2cc(-c3cccc(-c4ccc5c(c4)c4ccccc4n5-c4ccccc4)c3)ccc2-n2c3ccccc3c3ccccc32)cc1 ; Clc1ccc(-c2c(-c3ccc4c(c3)oc3ccccc34)cccc2-n2c3ccccc3c3ccccc32)cc1 ; Clc1ccc(-c2ccc(-c3ccc(-c4ccccc4)cc3)cc2-n2c3ccccc3c3ccccc32)cc1]

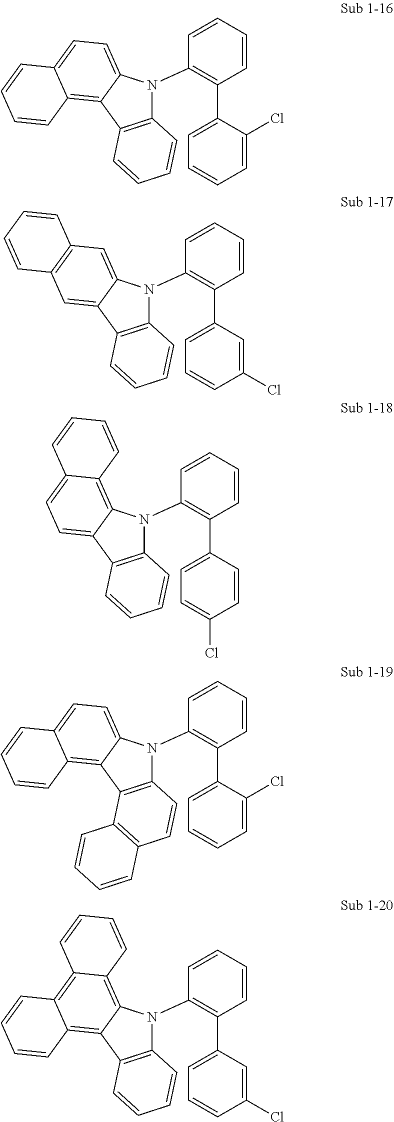

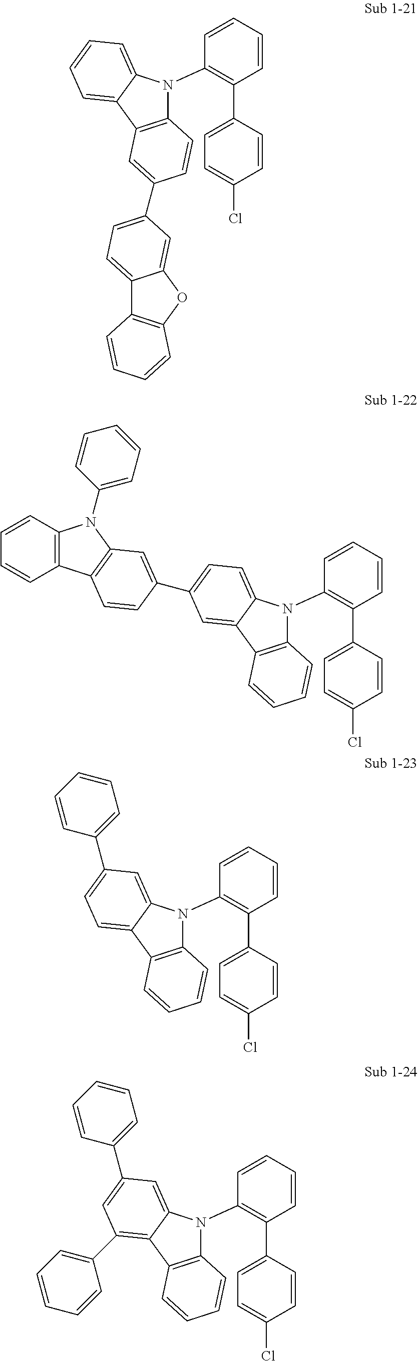 [SMILES: Clc1ccc(-c2ccccc2-n2c3ccccc3c3cc(-c4ccc5c(c4)oc4ccccc45)ccc32)cc1 ; Clc1ccc(-c2ccccc2-n2c3ccccc3c3cc(-c4ccc5c6ccccc6n(-c6ccccc6)c5c4)ccc32)cc1 ; Clc1ccc(-c2ccccc2-n2c3ccccc3c3ccc(-c4ccccc4)cc32)cc1 ; Clc1ccc(-c2ccccc2-n2c3ccccc3c3c(-c4ccccc4)cc(-c4ccccc4)cc32)cc1]

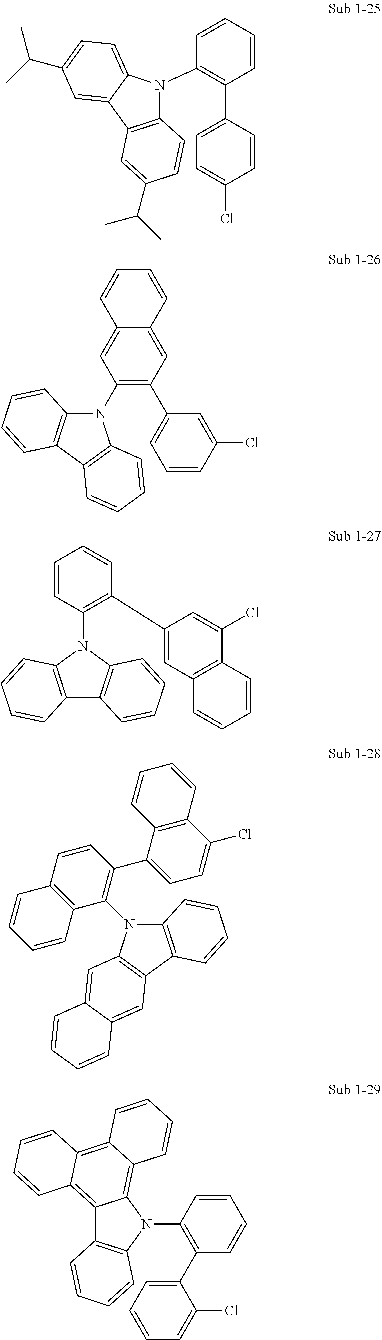

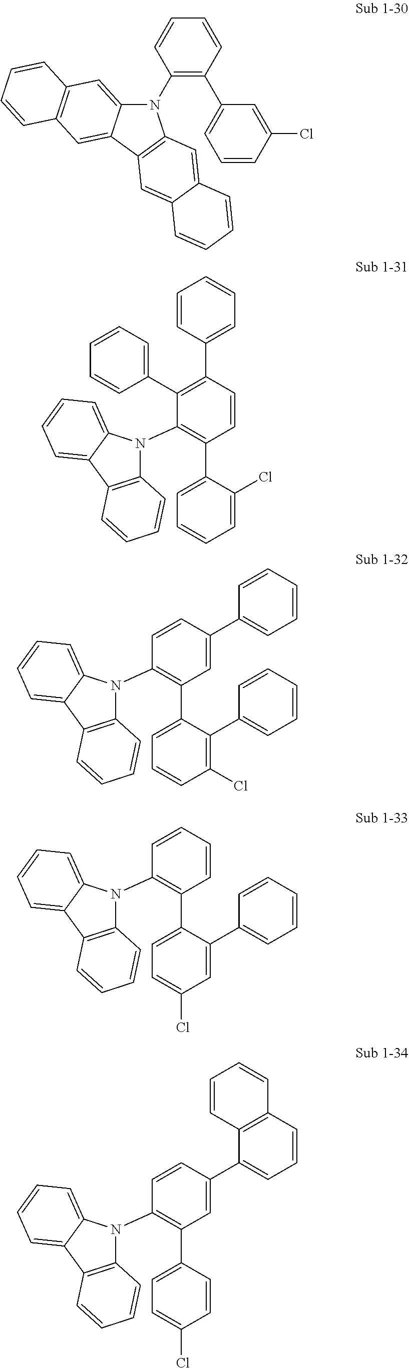

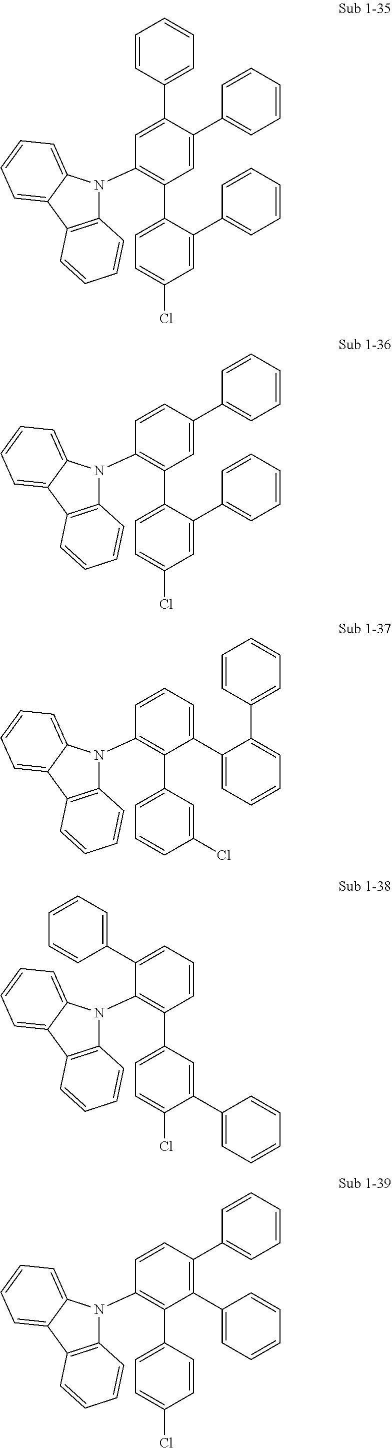

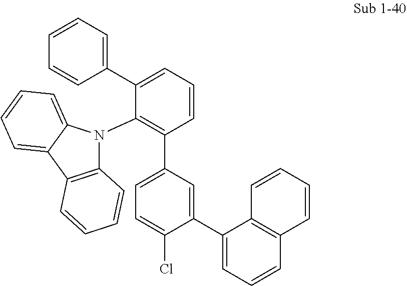 [SMILES: Clc1ccc(-c2cccc(-c3ccccc3)c2-n2c3ccccc3c3ccccc32)cc1-c1cccc2ccccc12]

TABLE 1

| compound | FD-MS | compound | FD-MS |
|---|---|---|---|
| Sub 1-1 | m/z = 353.10(C₂₄H₁₆ClN = 353.85) | Sub 1-2 | m/z = 353.10(C₂₄H₁₆ClN = 353.85) |
| Sub 1-3 | m/z = 353.10(C₂₄H₁₆ClN = 353.85) | Sub 1-4 | m/z = 429.13(C₃₀H₂₀ClN = 429.95) |
| Sub 1-5 | m/z = 505.16(C₃₆H₂₄ClN = 506.05) | Sub 1-6 | m/z = 429.13(C₃₀H₂₀ClN = 429.95) |
| Sub 1-7 | m/z = 519.14(C₃₆H₂₂ClNO = 520.03) | Sub 1-8 | m/z = 535.12(C₃₆H₂₂ClNS = 536.09) |
| Sub 1-9 | m/z = 518.15(C₃₆H₂₃ClN₂ = 519.04) | Sub 1-10 | m/z = 545.19(C₃₉H₂₈ClN = 546.11) |
| Sub 1-11 | m/z = 479.14(C₃₄H₂₂ClN = 480.01) | Sub 1-12 | m/z = 595.17(C₄₂H₂₆ClNO = 596.13) |
| Sub 1-13 | m/z = 670.22(C₄₈H₃₁ClN₂ = 671.24) | Sub 1-14 | m/z = 519.14(C₃₆H₂₂ClNO = 520.03) |
| Sub 1-15 | m/z = 505.16(C₃₆H₂₄ClN = 506.05) | Sub 1-16 | m/z = 403.11(C₂₈H₁₈ClN = 403.91) |
| Sub 1-17 | m/z = 403.11(C₂₈H₁₈ClN = 403.91) | Sub 1-18 | m/z = 403.11(C₂₈H₁₈ClN = 403.91) |
| Sub 1-19 | m/z = 453.13(C₃₂H₂₀ClN = 453.97) | Sub 1-20 | m/z = 453.13(C₃₂H₂₀ClN = 453.97) |
| Sub 1-21 | m/z = 519.14(C₃₆H₂₂ClNO = 520.03) | Sub 1-22 | m/z = 594.19(C₄₂H₂₇ClN₂ = 595.14) |
| Sub 1-23 | m/z = 429.13(C₃₀H₂₀ClN = 429.95) | Sub 1-24 | m/z = 505.16(C₃₆H₂₄ClN = 506.05) |
| Sub 1-25 | m/z = 437.19(C₃₀H₂₈ClN = 438.01) | Sub 1-26 | m/z = 403.11(C₂₈H₁₈ClN = 403.91) |
| Sub 1-27 | m/z = 403.11(C₂₈H₁₈ClN = 403.91) | Sub 1-28 | m/z = 503.14(C₃₆H₂₂ClN = 504.03) |
| Sub 1-29 | m/z = 453.13(C₃₂H₂₀ClN = 453.97) | Sub 1-30 | m/z = 453.13(C₃₂H₂₀ClN = 453.97) |
| Sub 1-31 | m/z = 505.16(C₃₆H₂₄ClN = 506.05) | Sub 1-32 | m/z = 505.16(C₃₆H₂₄ClN = 506.05) |
| Sub 1-33 | m/z = 429.13(C₃₀H₂₀ClN = 429.95) | Sub 1-34 | m/z = 479.14(C₃₄H₂₂ClN = 480.01) |
| Sub 1-35 | m/z = 581.19(C₄₂H₂₈ClN = 582.14) | Sub 1-36 | m/z = 505.16(C₃₆H₂₄ClN = 506.05) |
| Sub 1-37 | m/z = 505.16(C₃₆H₂₄ClN = 506.05) | Sub 1-38 | m/z = 505.16(C₃₆H₂₄ClN = 506.05) |
| Sub 1-39 | m/z = 505.16(C₃₆H₂₄ClN = 506.05) | Sub 1-40 | m/z = 555.18(C₄₀H₂₆ClN = 556.11) |

### II. Synthesis of Sub 2

Sub 2 of Reaction Scheme 1 is synthesized by the reaction pathway of Reaction Scheme 3, but is not limited thereto. Hal is I, Br or Cl.

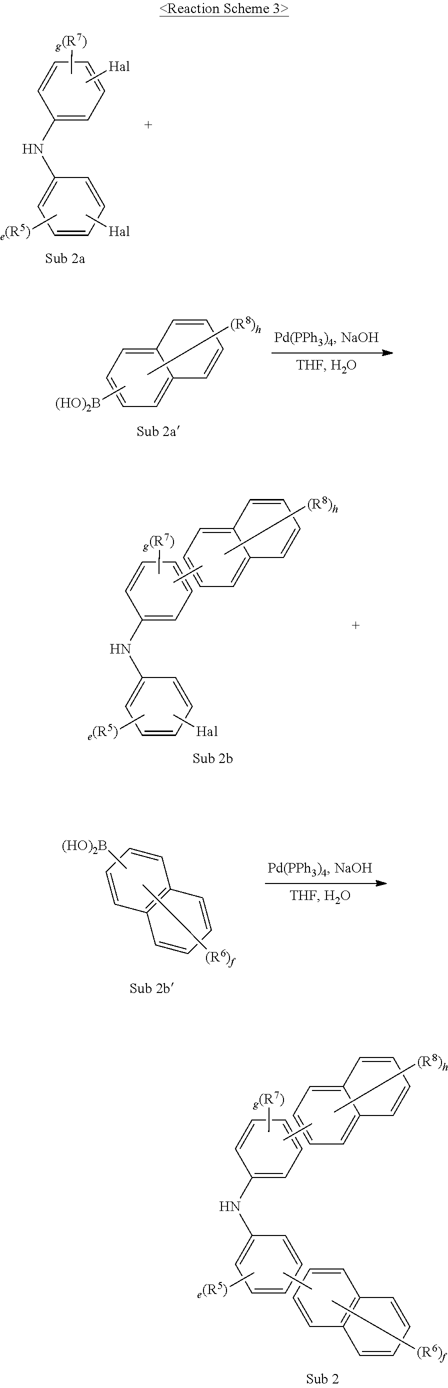

When Sub 2a′ and Sub 2b′ are the same, Sub 2 can be synthesized in one reaction.

### 1. Synthesis Example of Sub 2-2

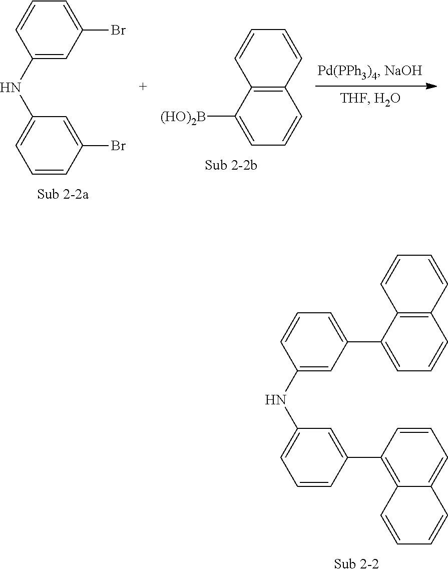 [SMILES: Brc1cccc(Nc2cccc(Br)c2)c1 ; OB(O)c1cccc2ccccc12 ; c1cc(Nc2cccc(-c3cccc4ccccc34)c2)cc(-c2cccc3ccccc23)c1]

After putting Sub 2-2a (50.0 g, 152.9 mmol) in a round bottom flask and dissolving in THF (764 ml), Sub 2-2b (52.6 g, 305.8 mmol), Pd(PPh₃)₄ (10.6 g, 9.2 mmol), NaOH (18.3 g, 458.7 mmol) and water (382 ml) were added and the reaction proceeded at 80° C. After the reaction was completed, extraction was performed with CH₂Cl₂ and water, and the organic layer was dried with MgSO₄, concentrated, and the resulting organic material was recrystallized using a silicagel column to obtain 53.9 g of the product. (Yield: 83.7%)

### 2. Synthesis Example of Sub 2-13

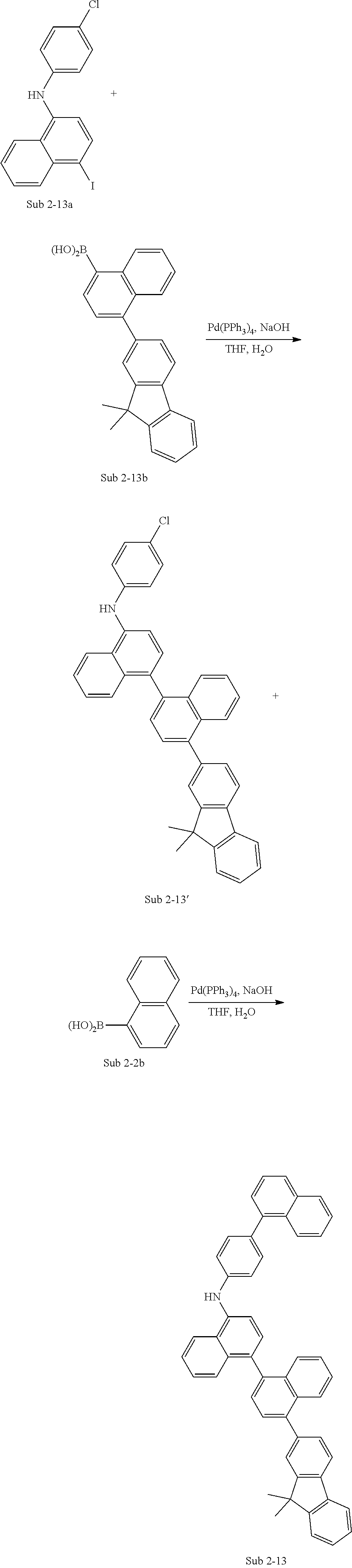 [SMILES: Clc1ccc(Nc2ccc(I)c3ccccc23)cc1 ; CC1(C)c2ccccc2-c2ccc(-c3ccc(B(O)O)c4ccccc34)cc21 ; CC1(C)c2ccccc2-c2ccc(-c3ccc(-c4ccc(Nc5ccc(Cl)cc5)c5ccccc45)c4ccccc34)cc21 ; OB(O)c1cccc2ccccc12 ; CC1(C)c2ccccc2-c2ccc(-c3ccc(-c4ccc(Nc5ccc(-c6cccc7ccccc67)cc5)c5ccccc45)c4ccccc34)cc21]

### (1) Synthesis of Sub 2-13′

After putting Sub 2-13a (50.0 g, 131.7 mmol) in a round bottom flask and dissolving in THF (659 ml), Sub 2-13b (48.0 g, 131.7 mmol), Pd(PPh₃)₄ (9.1 g, 7.9 mmol), NaOH (15.8 g, 395.1 mmol) and water (329 ml) were added and 61.1 g of the product was obtained in the same manner as in Sub 2-2. (Yield: 81.1%)

### (2) Synthesis of Sub 2-13

After putting Sub 2-13′ (61.1 g, 106.8 mmol) in a round bottom flask and dissolving in THF (534 ml), Sub 2-2b (18.4 g, 106.8 mmol), Pd(PPh₃)₄ (7.4 g, 6.4 mmol), NaOH (12.8 g, 320.4 mmol), water (267 ml) were added and 58.6 g of the product was obtained in the same manner as in Sub 2-2. (Yield: 80.2%)

The compound belonging to Sub 2 may be the following compounds, but is not limited thereto, and Table 2 below shows the FD-MS (Field Desorption-Mass Spectrometry) values of the compounds belonging to Sub 2.

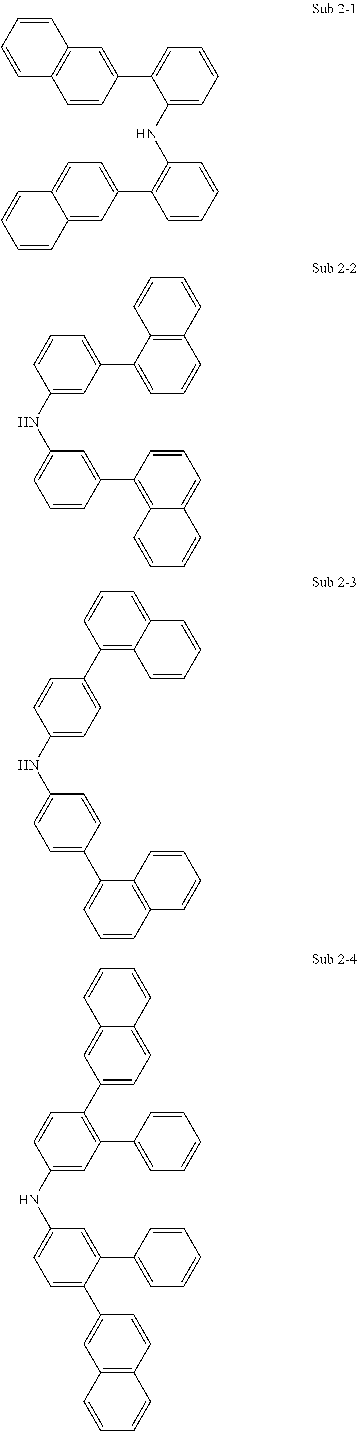

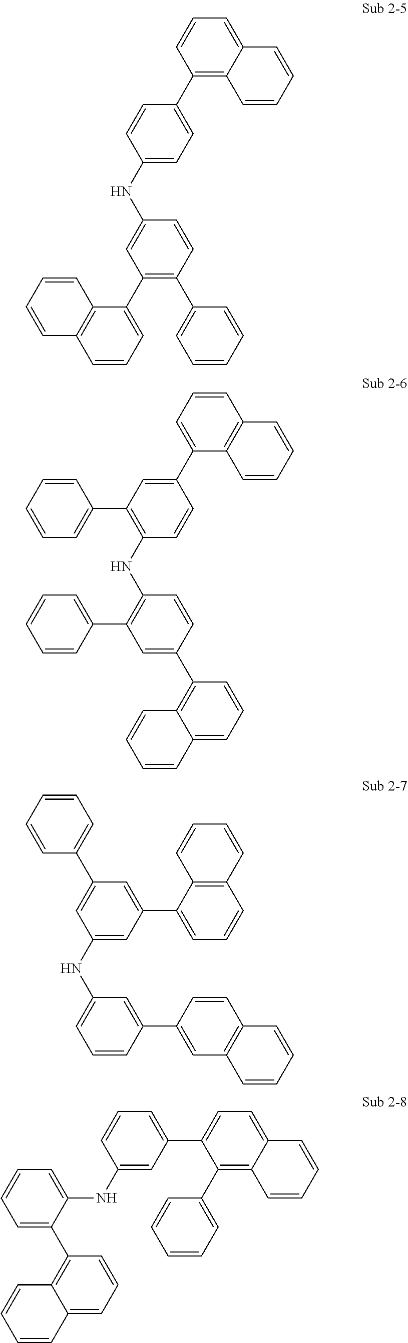

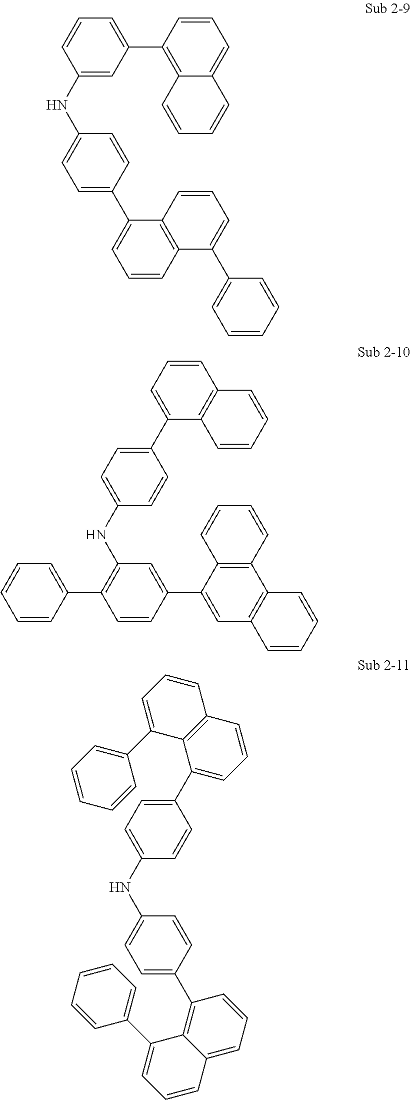

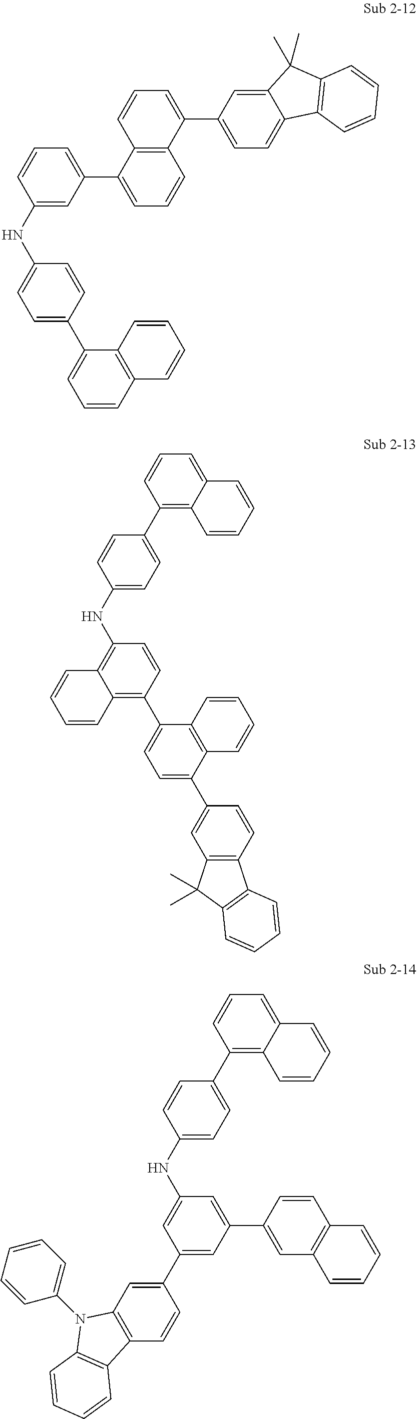

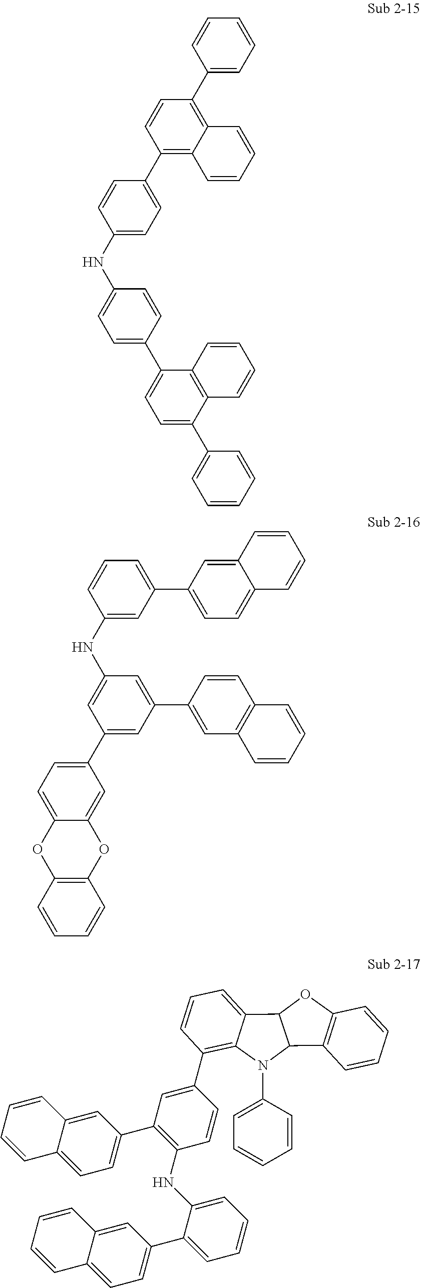

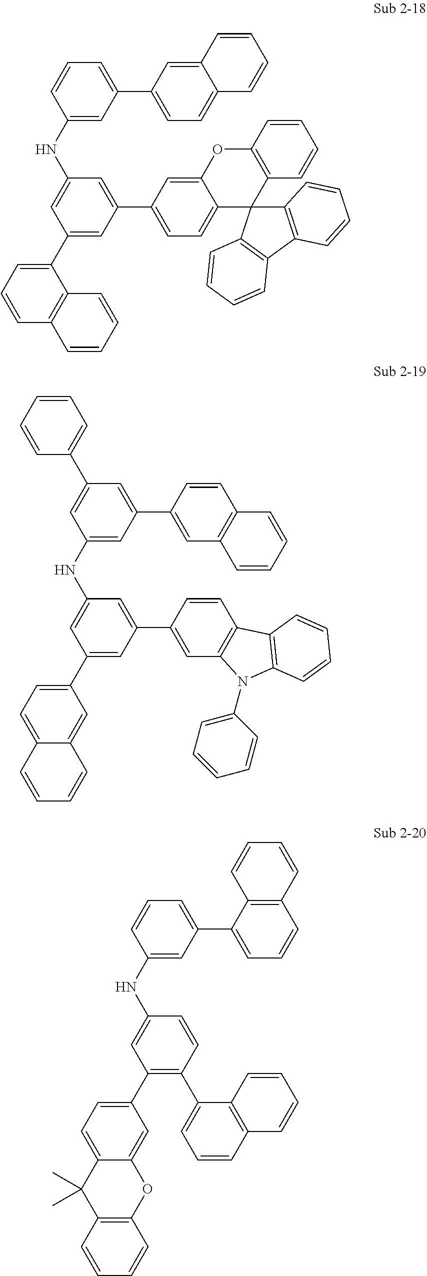

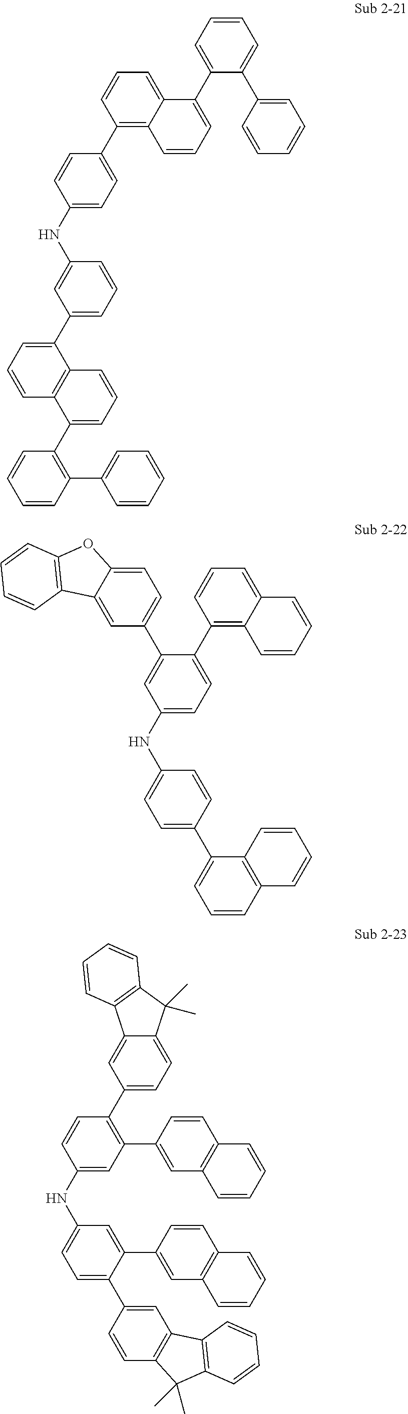

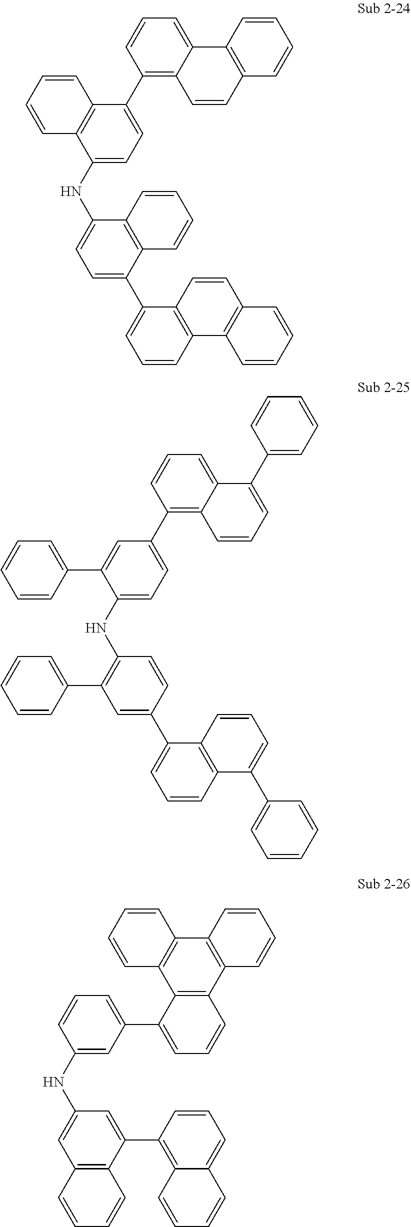

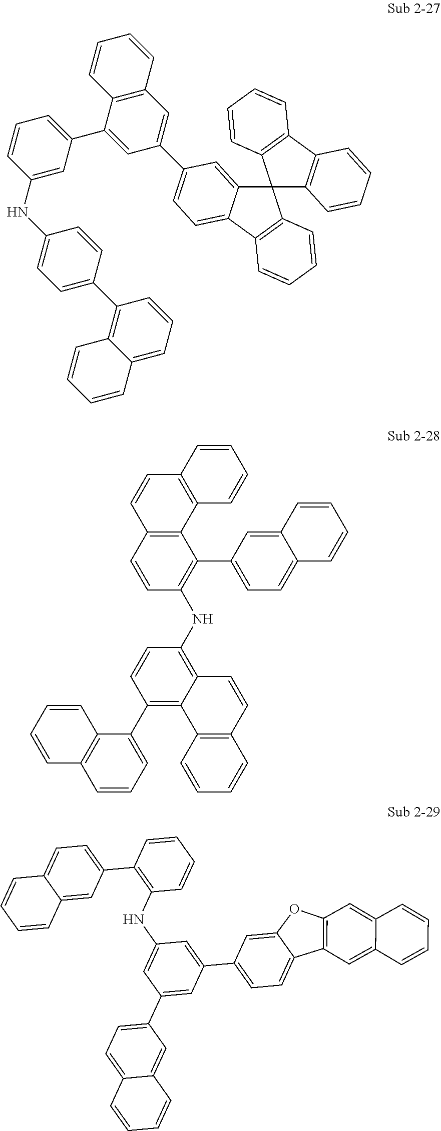 [SMILES: c1cc(Nc2ccc(-c3cccc4ccccc34)cc2)cc(-c2cc(-c3ccc4c(c3)C3(c5ccccc5-c5ccccc53)c3ccccc3-4)cc3ccccc23)c1 ; c1ccc2cc(-c3c(Nc4ccc(-c5cccc6ccccc56)c5c4ccc4ccccc45)ccc4ccc5ccccc5c34)ccc2c1 ; c1ccc(-c2ccc3ccccc3c2)c(Nc2cc(-c3ccc4ccccc4c3)cc(-c3ccc4c(c3)oc3cc5ccccc5cc34)c2)c1]

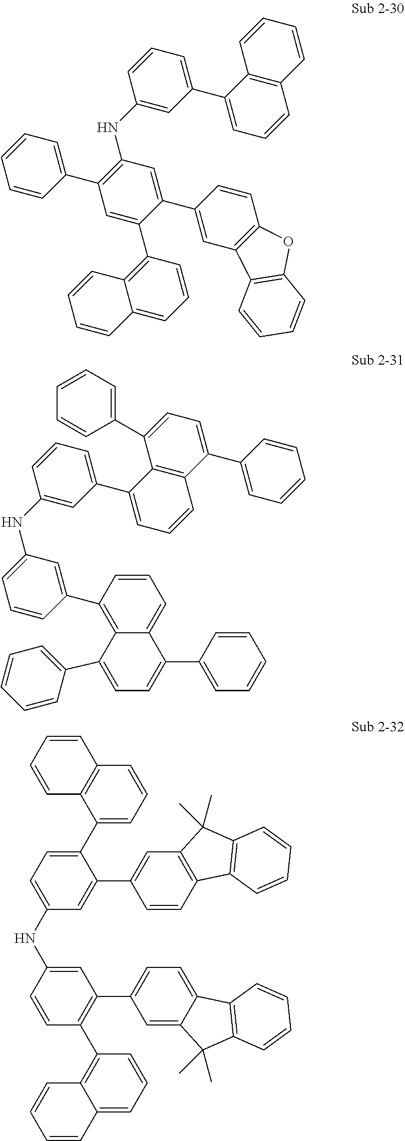

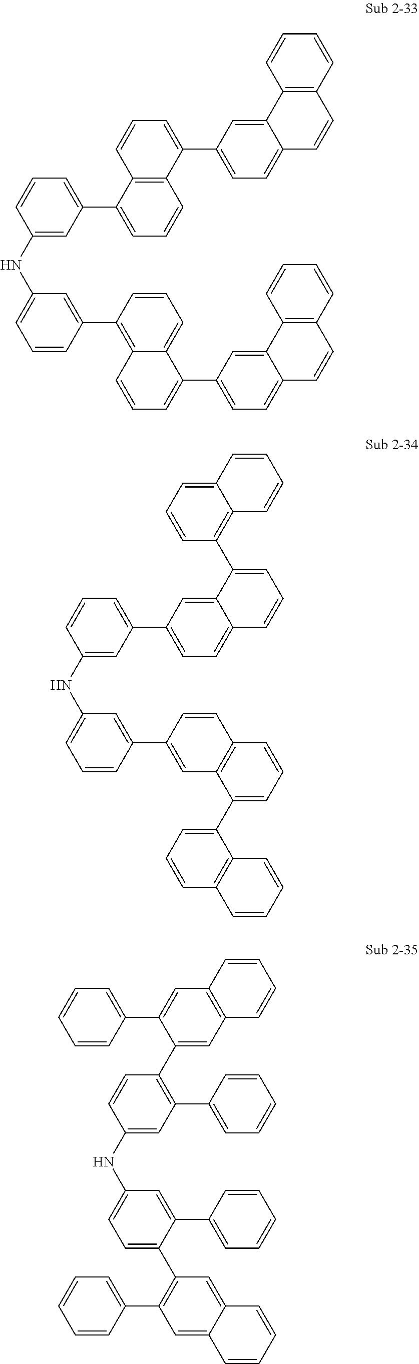

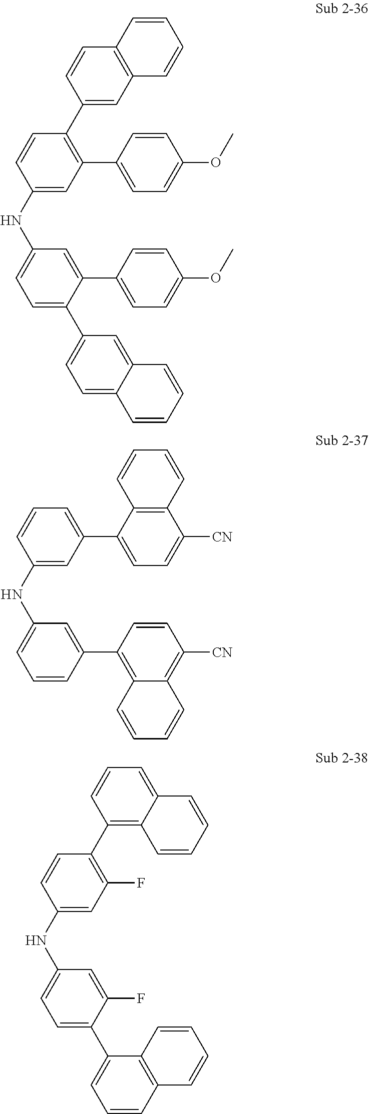

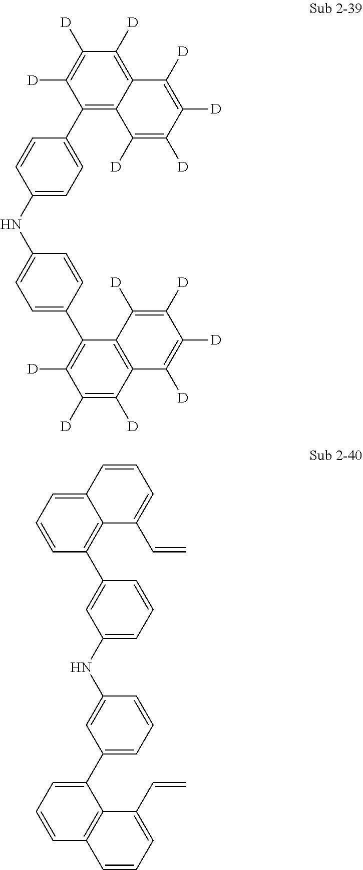

TABLE 2

| compound | FD-MS | compound | FD-MS |
|---|---|---|---|
| Sub 2-1 | m/z = 421.18(C₃₂H₂₃N = 421.54) | Sub 2-2 | m/z = 421.18(C₃₂H₂₃N = 421.54) |
| Sub 2-3 | m/z = 421.18(C₃₂H₂₃N = 421.54) | Sub 2-4 | m/z = 573.25(C₄₄H₃₁N = 573.74) |
| Sub 2-5 | m/z = 497.21(C₃₈H₂₇N = 497.64) | Sub 2-6 | m/z = 573.25(C₄₄H₃₁N = 573.74) |
| Sub 2-7 | m/z = 497.21(C₃₈H₂₇N = 497.64) | Sub 2-8 | m/z = 497.21(C₃₈H₂₇N = 497.64) |
| Sub 2-9 | m/z = 497.21(C₃₈H₂₇N = 497.64) | Sub 2-10 | m/z = 547.23(C₄₂H₂₉N = 547.70) |
| Sub 2-11 | m/z = 573.25(C₄₄H₃₁N = 573.74) | Sub 2-12 | m/z = 613.28(C₄₇H₃₅N = 613.80) |
| Sub 2-13 | m/z = 663.29(C₅₁H₃₇N = 663.86) | Sub 2-14 | m/z = 662.27(C₅₀H₃₄N₂ = 662.84) |
| Sub 2-15 | m/z = 573.25(C₄₄H₃₁N = 573.74) | Sub 2-16 | m/z = 603.22(C₄₄H₂₉NO₂ = 603.72) |
| Sub 2-17 | m/z = 702.27(C₅₂H₃₄N₂O = 702.86) | Sub 2-18 | m/z = 751.29(C₅₇H₃₇NO = 751.93) |
| Sub 2-19 | m/z = 738.30(C₅₆H₃₈N₂ = 738.93) | Sub 2-20 | m/z = 629.27(C₄₇H₃₅NO = 629.80) |
| Sub 2-21 | m/z = 725.31(C₅₆H₃₉N = 725.94) | Sub 2-22 | m/z = 587.22(C₄₄H₂₉NO = 587.72) |
| Sub 2-23 | m/z = 805.37(C₆₂H₄₇N = 806.07) | Sub 2-24 | m/z = 621.25(C₄₈H₃₁N = 621.78) |
| Sub 2-25 | m/z = 725.31(C₅₆H₃₉N = 725.94) | Sub 2-26 | m/z = 571.23(C₄₄H₂₉N = 571.72) |
| Sub 2-27 | m/z = 735.29(C₅₇H₃₇N = 735.93) | Sub 2-28 | m/z = 621.25(C₄₈H₃₁N = 621.78) |
| Sub 2-29 | m/z = 637.24(C₄₈H₃₁NO = 637.78) | Sub 2-30 | m/z = 663.26(C₅₀H₃₃NO = 663.82) |
| Sub 2-31 | m/z = 725.31(C₅₆H₃₉N = 725.94) | Sub 2-32 | m/z = 805.37(C₆₂H₄₇N = 806.07) |
| Sub 2-33 | m/z = 773.31(C₆₀H₃₉N = 773.98) | Sub 2-34 | m/z = 673.28(C₅₂H₃₅N = 673.86) |
| Sub 2-35 | m/z = 725.31(C₅₆H₃₉N = 725.94) | Sub 2-36 | m/z = 633.27(C₄₆H₃₅NO₂ = 633.79) |
| Sub 2-37 | m/z = 471.17(C₃₄H₂₁N₃ = 471.56) | Sub 2-38 | m/z = 457.16(C₃₂H₂₁F₂N = 457.52) |
| Sub 2-39 | m/z = 435.27(C₃₂H₉D₁₄N = 435.63) | Sub 2-40 | m/z = 473.21(C₃₆H₂₇N = 473.62) |

### III. Synthesis of Final Product

### 1. Synthesis Example of P-2

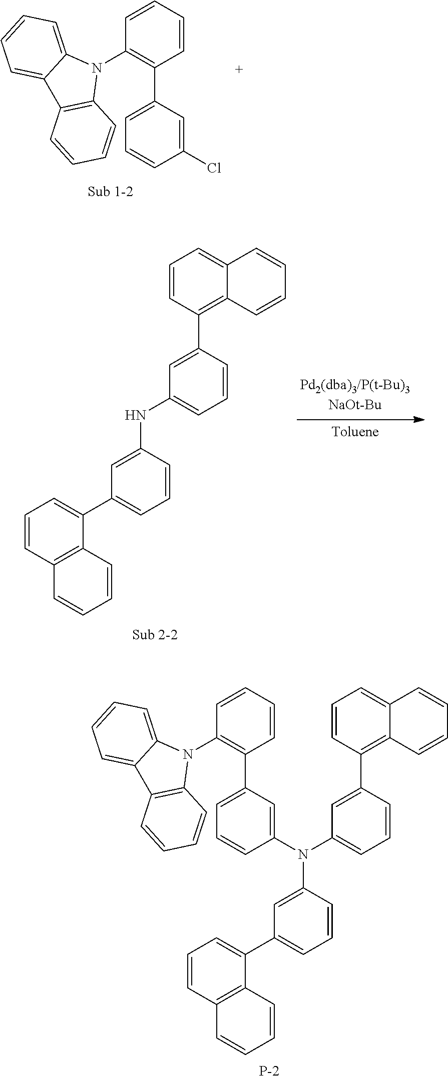

Sub 1-2 (10.0 g, 28.3 mmol), Sub 2-2 (11.9 g, 28.3 mmol), Pd₂(dba)₃ (0.8 g, 0.9 mmol), P(t-Bu)₃ (0.3 g, 1.7 mmol), NaOt-Bu (5.4 g, 56.5 mmol), Toluene (141 mL) were added to a round bottom flask in the same manner as in Sub 1-2 to obtain 15.3 g of product. (Yield: 73.4%)

### 2. Synthesis Example of P-3

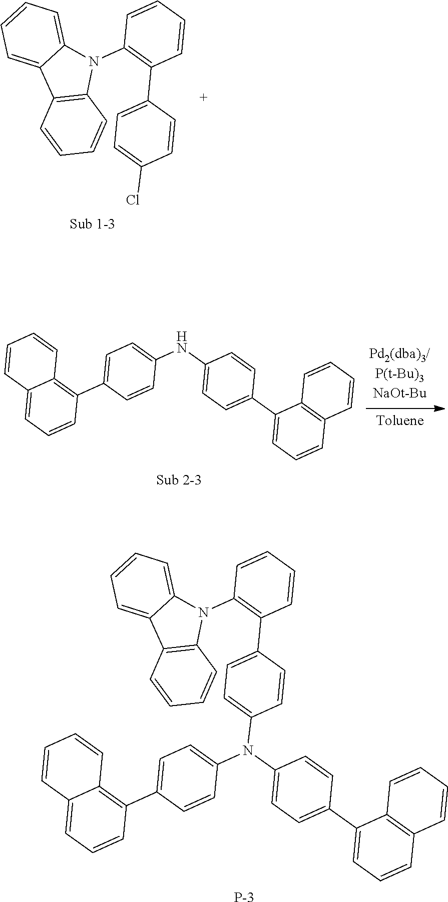

Sub 1-3 (10.0 g, 28.3 mmol), Sub 2-3 (11.9 g, 28.3 mmol), Pd₂(dba)₃ (0.8 g, mmol), P(t-Bu)₃ (0.3 g, 1.7 mmol), NaOt-Bu (5.4 g, 56.5 mmol), toluene (141 mL) were added to a round bottom flask in the same manner as in Sub 1-2 to obtain 15.4 g of product. (Yield: 73.8%)

### 3. Synthesis Example of P-18

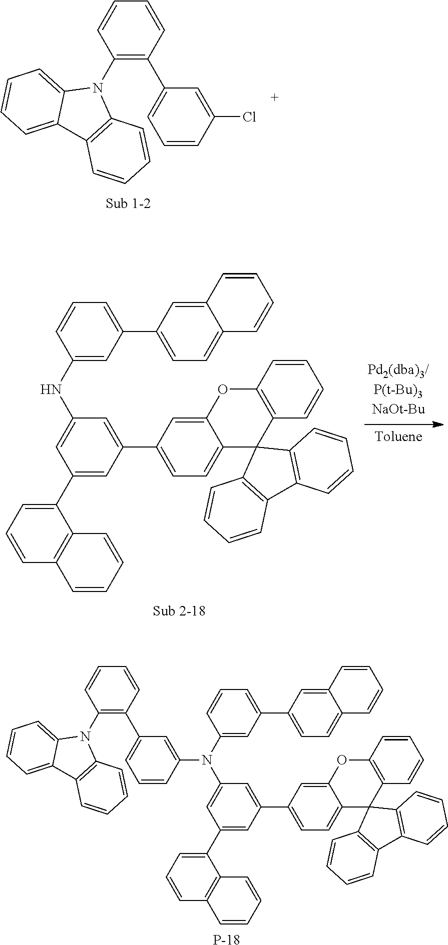

Sub 1-2 (10.0 g, 28.3 mmol), Sub 2-18 (21.2 g, 28.3 mmol), Pd₂(dba)₃ (0.8 g, mmol), P(t-Bu)₃ (0.3 g, 1.7 mmol), NaOt-Bu (5.4 g, 56.5 mmol), toluene (141 mL) were added to a round bottom flask in the same manner as in Sub 1-2 to obtain 20.8 g of product. (Yield: 68.7%)

### 4. Synthesis Example of P-39

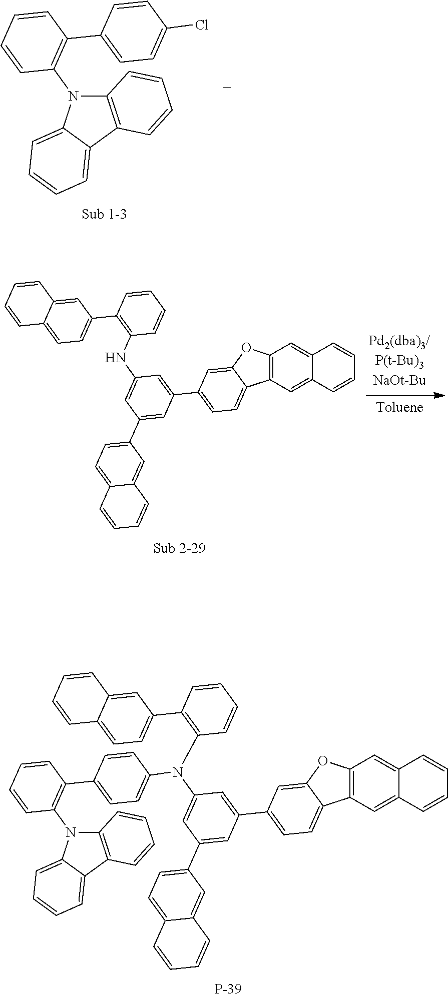

Sub 1-3 (10.0 g, 28.3 mmol), Sub 2-29 (18.0 g, 28.3 mmol), Pd₂(dba)₃ (0.8 g, mmol), P(t-Bu)₃ (0.3 g, 1.7 mmol), NaOt-Bu (5.4 g, 56.5 mmol), toluene (141 mL) were added to a round bottom flask in the same manner as in Sub 1-2 to obtain 15.2 g of product. (Yield: 71.2%)

### 5. Synthesis Example of P-78

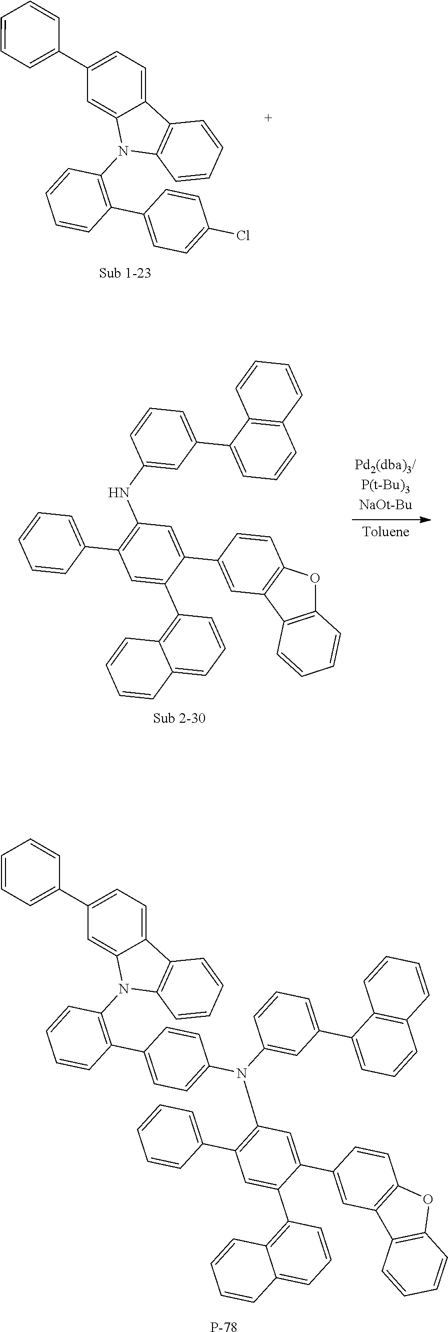

Sub 1-23 (10.0 g, 23.3 mmol), Sub 2-30 (15.4 g, 23.3 mmol), Pd₂(dba)₃ (0.6 g, mmol), P(t-Bu)₃ (0.3 g, 1.4 mmol), NaOt-Bu (4.5 g, 46.5 mmol), toluene (116 mL) were added to a round bottom flask in the same manner as in Sub 1-2 to obtain 17.4 g of product. (Yield: 70.7%)

### 6. Synthesis Example of P-86

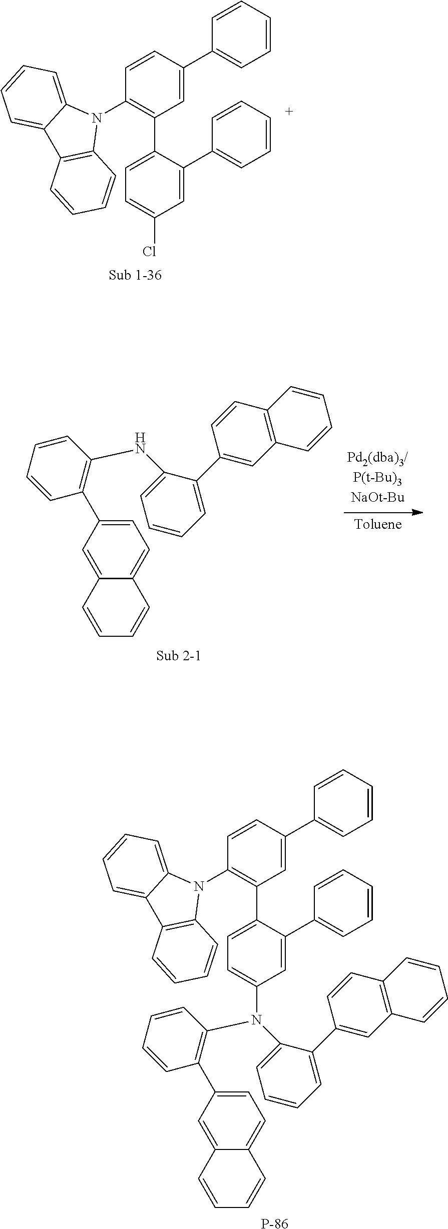

Sub 1-36 (10.0 g, 19.8 mmol), Sub 2-1 (8.3 g, 19.8 mmol), Pd₂(dba)₃ (0.5 g, mmol), P(t-Bu)₃ (0.2 g, 1.2 mmol), NaOt-Bu (3.8 g, 39.5 mmol), toluene (99 mL) were added to a round bottom flask in the same manner as in Sub 1-2 to obtain 12.8 g of product. (Yield: 72.5%)

The FD-MS values of the compounds P-1 to P-90 of the present invention prepared according to the Synthesis Example as described above are shown in Table 3.

TABLE 3

| compound | FD-MS | compound | FD-MS |
|---|---|---|---|
| P-1 | m/z = 738.30(C₅₆H₃₈N₂ = 738.93) | P-2 | m/z = 738.30(C₅₆H₃₈N₂ = 738.93) |
| P-3 | m/z = 738.30(C₅₆H₃₈N₂ = 738.93) | P-4 | m/z = 966.40(C₇₄H₅₀N₂ = 967.23) |
| P-5 | m/z = 814.33(C₆₂H₄₂N₂ = 815.03) | P-6 | m/z = 890.37(C₆₈H₄₆N₂ = 891.13) |
| P-7 | m/z = 814.33(C₆₂H₄₂N₂ = 815.03) | P-8 | m/z = 966.40(C₇₄H₅₀N₂ = 967.23) |
| P-9 | m/z = 814.33(C₆₂H₄₂N₂ = 815.03) | P-10 | m/z = 864.35(C₆₆H₄₄N₂ = 865.09) |
| P-11 | m/z = 890.37(C₆₈H₄₆N₂ = 891.13) | P-12 | m/z = 1006.43(C₇₇H₅₄N₂ = 1007.29) |
| P-13 | m/z = 980.41(C₇₅H₅₂N₂ = 981.26) | P-14 | m/z = 979.39(C₇₄H₄₉N₃ = 980.23) |
| P-15 | m/z = 890.37(C₆₈H₄₆N₂ = 891.13) | P-16 | m/z = 1086.38(C₈₀H₅₀N₂O₃ = 1087.29) |
| P-17 | m/z = 1019.39(C₇₆H₄₉N₃O = 1020.25) | P-18 | m/z = 1068.41(C₈₁H₅₂N₂O = 1069.32) |
| P-19 | m/z = 1055.42(C₈₀H₅₃N₃ = 1056.33) | P-20 | m/z = 1128.41(C₈₃H₅₆N₂OS = 1129.43) |
| P-21 | m/z = 1042.43(C₈₀H₅₄N₂ = 1043.33) | P-22 | m/z = 904.35(C₆₈H₄₄N₂O = 905.11) |
| P-23 | m/z = 1122.49(C₈₆H₆₂N₂ = 1123.46) | P-24 | m/z = 1103.42(C₈₄H₅₃N₃ = 1104.37) |
| P-25 | m/z = 1042.43(C₈₀H₅₄N₂ = 1043.33) | P-26 | m/z = 920.34(C₆₈H₄₄N₂O₂ = 921.11) |
| P-27 | m/z = 1019.39(C₇₆H₄₉N₃O = 1020.25) | P-28 | m/z = 1260.50(C₉₆H₆₄N₂O = 1261.58) |
| P-29 | m/z = 1055.42(C₈₀H₅₃N₃ = 1056.33) | P-30 | m/z = 946.39(C₇₁H₅₀N₂O = 947.19) |
| P-31 | m/z = 1042.43(C₈₀H₅₄N₂ = 1043.33) | P-32 | m/z = 1030.39(C₇₈H₅₀N₂O = 1031.27) |
| P-33 | m/z = 1122.49(C₈₆H₆₂N₂ = 1123.46) | P-34 | m/z = 1068.42(C₈₀H₅₂N₄ = 1069.32) |
| P-35 | m/z = 1042.43(C₈₀H₅₄N₂ = 1043.33) | P-36 | m/z = 1130.42(C₈₆H₅₄N₂O = 1131.39) |
| P-37 | m/z = 1052.41(C₈₁H₅₂N₂ = 1053.32) | P-38 | m/z = 938.37(C₇₂H₄₆N₂ = 939.17) |
| P-39 | m/z = 954.36(C₇₂H₄₆N₂O = 955.17) | P-40 | m/z = 1297.50(C₉₈H₆₃N₃O = 1298.60) |
| P-41 | m/z = 1042.43(C₈₀H₅₄N₂ = 1043.33) | P-42 | m/z = 1122.49(C₈₆H₆₂N₂ = 1123.46) |
| P-43 | m/z = 1090.43(C₈₄H₅₄N₂ = 1091.37) | P-44 | m/z = 1156.44(C₈₈H₅₆N₂O = 1157.43) |
| P-45 | m/z = 1042.43(C₈₀H₅₄N₂ = 1043.33) | P-46 | m/z = 950.39(C₇₀H₅₀N₂O₂ = 951.18) |
| P-47 | m/z = 788.29(C₅₈H₃₆N₄ = 788.95) | P-48 | m/z = 926.35(C₆₈H₄₄F₂N₂ = 927.11) |
| P-49 | m/z = 752.39(C₅₆H₂₄D₁₄N₂ = 753.02) | P-50 | m/z = 790.33(C₆₀H₄₂N₂ = 791.01) |
| P-51 | m/z = 738.30(C₅₆H₃₈N₂ = 738.93) | P-52 | m/z = 864.35(C₆₆H₄₄N₂ = 865.09) |
| P-53 | m/z = 890.37(C₆₈H₄₆N₂ = 891.13) | P-54 | m/z = 814.33(C₆₂H₄₂N₂ = 815.03) |
| P-55 | m/z = 814.33(C₆₂H₄₂N₂ = 815.03) | P-56 | m/z = 788.32(C₆₀H₄₀N₂ = 788.99) |
| P-57 | m/z = 890.37(C₆₈H₄₆N₂ = 891.13) | P-58 | m/z = 890.37(C₆₈H₄₆N₂ = 891.13) |
| P-59 | m/z = 1042.43(C₈₀H₅₄N₂ = 1043.33) | P-60 | m/z = 988.38(C₇₆H₄₈N₂ = 989.23) |
| P-61 | m/z = 1052.41(C₈₁H₅₂N₂ = 1053.32) | P-62 | m/z = 788.32(C₆₀H₄₀N₂ = 788.99) |
| P-63 | m/z = 954.36(C₇₂H₄₆N₂O = 955.17) | P-64 | m/z = 1190.46(C₉₂H₅₈N₂ = 1191.49) |
| P-65 | m/z = 1090.43(C₈₄H₅₄N₂ = 1091.37) | P-66 | m/z = 1122.49(C₈₆H₆₂N₂ = 1123.46) |
| P-67 | m/z = 788.32(C₆₀H₄₀N₂ = 788.99) | P-68 | m/z = 838.33(C₆₄H₄₂N₂ = 839.05) |
| P-69 | m/z = 921.17(C₆₈H₄₄N₂S = 921.17) | P-70 | m/z = 1042.43(C₈₀H₅₄N₂ = 1043.33) |
| P-71 | m/z = 888.35(C₆₈H₄₄N₂ = 889.11) | P-72 | m/z = 904.35(C₆₈H₄₄N₂O = 905.11) |
| P-73 | m/z = 838.33(C₆₄H₄₂N₂ = 839.05) | P-74 | m/z = 838.33(C₆₄H₄₂N₂ = 839.05) |
| P-75 | m/z = 890.37(C₆₈H₄₆N₂ = 891.13) | P-76 | m/z = 1131.46(C₈₆H₅₇N₃ = 1132.42) |
| P-77 | m/z = 814.33(C₆₂H₄₂N₂ = 815.03) | P-78 | m/z = 1056.41(C₈₀H₅₂N₂O = 1057.31) |
| P-79 | m/z = 890.37(C₆₈H₄₆N₂ = 891.13) | P-80 | m/z = 1152.50(C₈₇H₆₄N₂O = 1153.48) |
| P-81 | m/z = 1042.43(C₈₀H₅₄N₂ = 1043.33) | P-82 | m/z = 1194.49(C₉₂H₆₂N₂ = 1195.52) |
| P-83 | m/z = 814.33(C₆₂H₄₂N₂ = 815.03) | P-84 | m/z = 1016.41(C₇₈H₅₂N₂ = 1017.29) |
| P-85 | m/z = 1118.46(C₈₆H₅₈N₂ = 1119.42) | P-86 | m/z = 890.37(C₆₈H₄₆N₂ = 891.13) |
| P-87 | m/z = 890.37(C₆₈H₄₆N₂ = 891.13) | P-88 | m/z = 890.37(C₆₈H₄₆N₂ = 891.13) |
| P-89 | m/z = 1220.47(C₉₃H₆₀N₂O = 1221.52) | P-90 | m/z = 940.38(C₇₂H₄₈N₂ = 941.19) |

### Synthesis Example 2

The synthesis example of Product 8-6 among the compounds represented by Formula 2 according to the present invention is shown in Reaction Scheme 4.

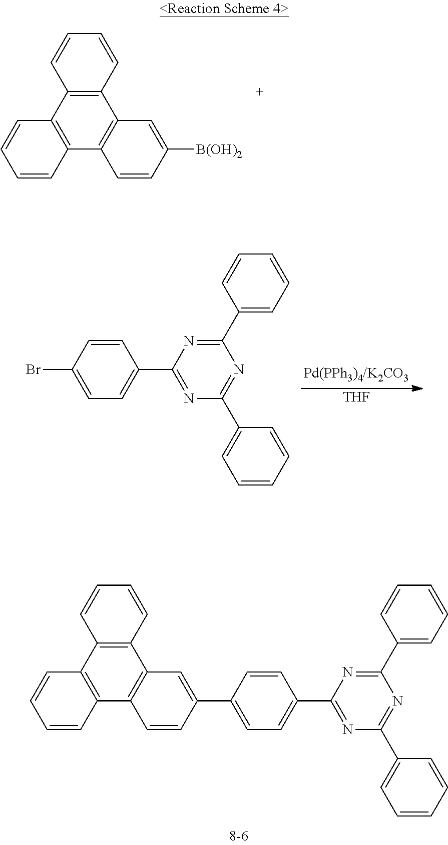 [SMILES: OB(O)c1ccc2c3ccccc3c3ccccc3c2c1 ; Brc1ccc(-c2nc(-c3ccccc3)nc(-c3ccccc3)n2)cc1 ; c1ccc(-c2nc(-c3ccccc3)nc(-c3ccc(-c4ccc5c6ccccc6c6ccccc6c5c4)cc3)n2)cc1 ; "THF"]

After dissolving triphenylen-2-ylboronic acid (5.4 g, 20 mmol) in THF, adding 2-(4-bromophenyl)-4,6-diphenyl-1,3,5-triazine (7.8 g, 20 mmol), Pd(PPh₃)₄ (0.03 equiv.), K₂CO₃ (3 equiv.) and water, and stir and reflux. When the reaction was completed, the mixture was extracted with ether and water, and the organic layer was dried over MgSO₄, concentrated, and the organic material was recrystallized using a silica gel column to obtain 7.5 g of the product. (Yield: 70%)

FD-MS values of compounds 8-1 to 8-18 of the present invention are shown in Table 4.

TABLE 4

| compound | FD-MS | compound | FD-MS |
|---|---|---|---|
| 8-1 | m/z = 401.11(C₃₀H₁₈S = 410.53) | 8-2 | m/z = 401.11(C₃₀H₁₈S = 410.53) |
| 8-3 | m/z = 486.14(C₃₆H₂₂S = 486.62) | 8-4 | m/z = 486.14(C₃₆H₂₂S = 486.62) |
| 8-5 | m/z = 534.21(C₄₀H₂₆N₂ = 534.65) | 8-6 | m/z = 535.20(C₃₉H₂₅N₃ = 535.64) |
| 8-7 | m/z = 536.16(C₄₀H₂₄S = 536.68) | 8-8 | m/z = 536.16(C₄₀H₂₄S = 536.68) |
| 8-9 | m/z = 575.17(C₄₂H₂₅NS = 575.72) | 8-10 | m/z = 559.19(C₄₂H₂₅NO = 559.65) |
| 8-11 | m/z = 585.25(C₄₅H₃₁N = 585.73) | 8-12 | m/z = 575.17(C₄₂H₂₅NS = 575.72) |
| 8-13 | m/z = 304.13(C₂₄H₁₆ = 304.39) | 8-14 | m/z = 354.14(C₂₈H₁₈ = 354.45) |
| 8-15 | m/z = 354.14(C₂₈H₁₈ = 354.45) | 8-16 | m/z = 380.16(C₃₀H₂₀ = 380.49) |
| 8-17 | m/z = 630.23(C₅₀H₃₀ = 630.79) | 8-18 | m/z = 454.17(C₃₆H₂₂ = 454.57) |

### Synthesis Example 3

An example of the synthesis of Product 9-10 among the compounds represented by Formula 3 according to the present invention is shown in Reaction Scheme 5.

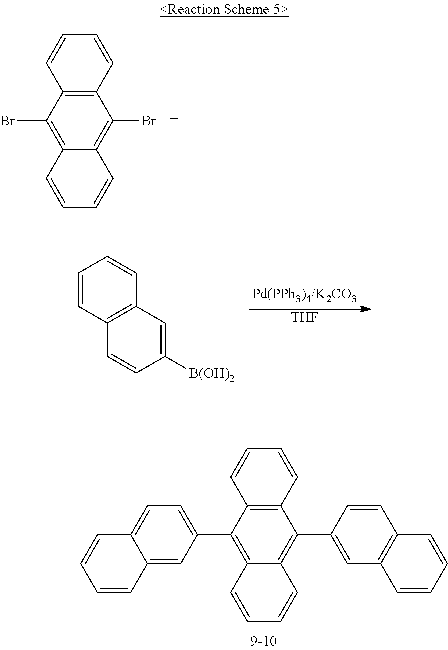

After dissolving 9,10-dibromoanthracene (6.7 g, 20 mmol) in THF, adding naphthalen-2-ylboronic acid (7.6g, 44 mmol), Pd(PPh₃)₄ (0.03 equiv.), K₂CO₃ (3 equiv.) and water, and stir and reflux. When the reaction was completed, the mixture was extracted with ether and water, and the organic layer was dried over MgSO₄, concentrated, and the organic material was recrystallized using a silica gel column to obtain 6.7 g of the product. (Yield: 78%)

FD-MS values of compounds 9-1 to 9-32 of the present invention are shown in Table 5.

TABLE 5

| compound | FD-MS | compound | FD-MS |
|---|---|---|---|
| 9-1 | m/z = 330.14(C₂₆H₁₈ = 330.42) | 9-2 | m/z = 380.16(C₃₀H₂₀ = 380.48) |
| 9-3 | m/z = 380.16(C₃₀H₂₀ = 380.48) | 9-4 | m/z = 406.17(C₃₂H₂₂ = 406.52) |
| 9-5 | m/z = 454.17(C₃₆H₂₂ = 454.56) | 9-6 | m/z = 430.17(C₃₄H₂₂ = 430.54) |
| 9-7 | m/z = 430.17(C₃₄H₂₂ = 430.54) | 9-8 | m/z = 456.19(C₃₆H₂₄ = 456.58) |
| 9-9 | m/z = 504.19(C₄₀H₂₄ = 504.62) | 9-10 | m/z = 430.17(C₃₄H₂₂ = 430.54) |
| 9-11 | m/z = 456.19(C₃₆H₂₄ = 456.58) | 9-12 | m/z = 486.14(C₃₆H₂₂S = 486.62) |
| 9-13 | m/z = 482.20(C₃₈H₂₆ = 482.61) | 9-14 | m/z = 561.22(C₄₁H₂₇N₃ = 561.67) |
| 9-15 | m/z = 506.20(C₄₀H₂₆ = 506.63) | 9-16 | m/z = 582.23(C₄₈H₃₀ = 582.73) |
| 9-17 | m/z = 406.17(C₃₂H₂₂ = 406.52) | 9-18 | m/z = 506.20(C₄₀H₂₆ = 506.63) |
| 9-19 | m/z = 506.20(C₄₀H₂₆ = 506.63) | 9-20 | m/z = 482.20(C₃₈H₂₆ = 482.61) |
| 9-21 | m/z = 512.23(C₃₈H₂₈N₂ = 512.64) | 9-22 | m/z = 664.29(C₅₀H₃₆N₂ = 664.83) |
| 9-23 | m/z = 568.29(C₄₂H₃₆N₂ = 568.75) | 9-24 | m/z = 740.32(C₅₆H₄₀N₂ = 740.93) |
| 9-25 | m/z = 420.15(C₃₂H₂₀O = 420.51) | 9-26 | m/z = 600.17(C₄₄H₂₄O₃ = 600.67) |
| 9-27 | m/z = 595.23(C₄₆H₂₉N = 595.75) | 9-28 | m/z = 586.19(C₄₄H₂₆O₂ = 586.69) |
| 9-29 | m/z = 420.15(C₃₂H₂₀O = 420.51) | 9-30 | m/z = 496.18(C₃₈H₂₄O = 496.61) |
| 9-31 | m/z = 496.18(C₃₈H₂₄O = 496.61) | 9-32 | m/z = 520.18(C₄₀H₂₄O = 520.63) |

### Synthesis Example 4

An example of the synthesis of Product 10-14 among the compounds represented by Formula 4 according to the present invention is shown in Reaction Scheme 6.

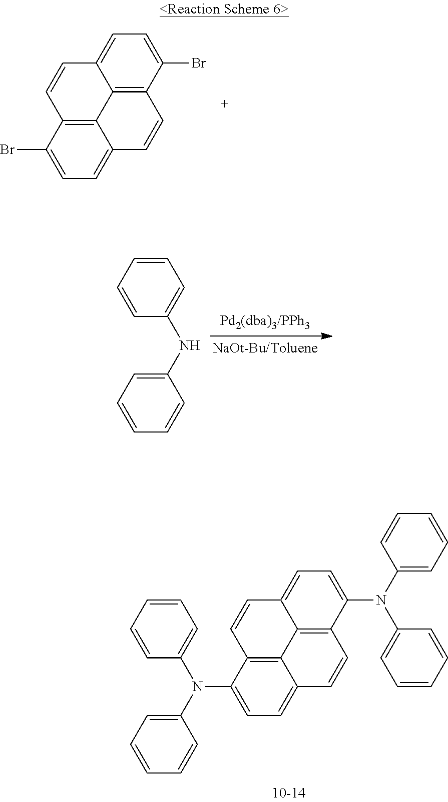 [SMILES: Brc1ccc2ccc3c(Br)ccc4ccc1c2c43 ; c1ccc(Nc2ccccc2)cc1 ; c1ccc(N(c2ccccc2)c2ccc3ccc4c(N(c5ccccc5)c5ccccc5)ccc5ccc2c3c54)cc1]

After adding 1,6-dibromo-1,6-dihydropyrene (7.2 g, 20 mmol), diphenylamine (7.4 g, 44 mmol), Pd₂(dba)₃ (0.03-0.05 mmol), P(t-Bu)₃ (0.1 equiv.), NaOt-Bu (3 equiv.), toluene (10.5 mL/1 mmol) to a round bottom flask, the reaction is carried out at 100° C. When the reaction was completed, the mixture was extracted with ether and water, and the organic layer was dried over MgSO₄, concentrated, and the organic material was recrystallized using a silica gel column to obtain 8.1 g of the product. (Yield: 75%)

FD-MS values of compounds 10-1 to 10-20 of the present invention are shown in Table 6.

TABLE 6

| compound | FD-MS | compound | FD-MS |
|---|---|---|---|
| 10-1 | m/z = 354.14(C₂₈H₁₈ = 354.44) | 10-2 | m/z = 454.17(C₃₆H₂₂ = 454.56) |
| 10-3 | m/z = 454.17(C₃₆H₂₂ = 454.56) | 10-4 | m/z = 356.13(C₂₆H₁₆N₂ = 356.42) |
| 10-5 | m/z = 354.14(C₂₈H₁₈ = 354.44) | 10-6 | m/z = 454.17(C₃₆H₂₂ = 454.56) |
| 10-7 | m/z = 454.17(C₃₆H₂₂ = 454.56) | 10-8 | m/z = 356.13(C₂₆H₁₆N₂ = 356.42) |
| 10-9 | m/z = 354.14(C₂₈H₁₈ = 354.44) | 10-10 | m/z = 454.17(C₃₆H₂₂ = 454.56) |
| 10-11 | m/z = 454.17(C₃₆H₂₂ = 454.56) | 10-12 | m/z = 356.13(C₂₆H₁₆N₂ = 356.42) |
| 10-13 | m/z = 536.23(C₄₀H₂₈N₂ = 536.66) | 10-14 | m/z = 536.23(C₄₀H₂₈N₂ = 536.66) |
| 10-15 | m/z = 536.23(C₄₀H₂₈N₂ = 536.66) | 10-16 | m/z = 804.33(C₅₈H₄₂F₂N₂ = 804.96) |
| 10-17 | m/z = 716.25(C₅₂H₃₂N₂O₂ = 716.84) | 10-18 | m/z = 768.35(C₅₈H₄₄N₂ = 769.00) |
| 10-19 | m/z = 848.23(C₆₀H₃₆N₂S₂ = 849.08) | 10-20 | m/z = 868.31(C₆₄H₄₀N₂O₂ = 869.04) |

### Manufacturing Evaluation of Organic Electronic Elements

### Example 1 Blue Organic Light Emitting Device

An organic light emitting device was manufactured according to a conventional method using the compound of the present invention as the material for the emitting auxiliary layer. First, N¹-(naphthalen-2-yl)-N⁴,N⁴-bis(4-(naphthalen-2-yl(phenyl)amino)phenyl)-N¹-phenylbenzene-1,4-diamine (hereinafter abbreviated as 2-TNATA) film was vacuum-deposited as a hole injection layer on an ITO layer (anode) formed on a glass substrate to have a thickness of 60 nm, and then N,N′-bis(1-naphthalenyl)-N,N′-bis-phenyl-(1,1′-biphenyl)-4,4′-diamine (hereinafter abbreviated as NPB) film was vacuum-deposited to a thickness of 60 nm to form a hole transport layer.

Thereafter, on the hole transport layer, the compound P-3 of the present invention was vacuum deposited to a thickness of 20 nm to form an emitting auxiliary layer, and on the emitting auxiliary layer, 9,10-di(naphthalen-2-yl)anthracene as a host and BD-052X (Idemitsu kosan) as a dopant were used, but the dopant was doped so that the weight ratio of these was 96:4, and a 30 nm thick of emitting layer was deposited.

Next, a hole blocking layer was formed by vacuum depositing (1,1′-biphenyl-4-olato)bis(2-methyl-8-quinolinolato)aluminum (hereinafter abbreviated as BAlq) to a thickness of 10 nm on the emitting layer, and an electron transport layer was formed by depositing tris(8-quinolinol)aluminum (hereinafter abbreviated as Alq₃) to a thickness of 40 nm on the hole blocking layer.

Thereafter, LiF was deposited on the electron transport layer to a thickness of nm to form an electron injection layer, and Al was deposited on the electron injection layer to a thickness of 150 nm to form a cathode.

### Example 2 to Example 20

An organic electroluminescent device was manufactured in the same manner as in Example 1, except that the compound of the present invention shown in Table 7 was used instead of the compound P-3 of the present invention as a material for the emitting auxiliary layer.

### Comparative Example 1

An organic light emitting device was manufactured in the same manner as in Example 1 except that the emitting auxiliary layer was not formed.

### Comparative Example 2 and Comparative Example 3

An organic electroluminescent device was manufactured in the same manner as in Example 1, except that Comparative Compound A and Comparative Compound B were used instead of Compound P-3 of the present invention as a material for the emitting auxiliary layer.

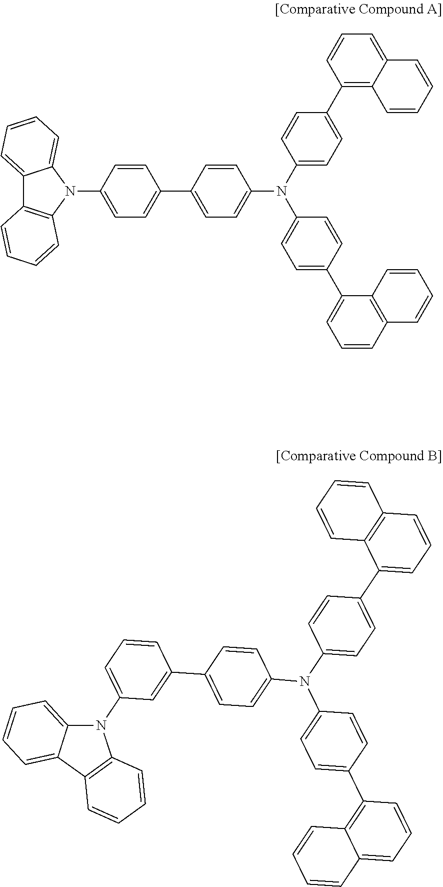 [SMILES: c1ccc2c(-c3ccc(N(c4ccc(-c5ccc(-n6c7ccccc7c7ccccc76)cc5)cc4)c4ccc(-c5cccc6ccccc56)cc4)cc3)cccc2c1 ; c1cc(-c2ccc(N(c3ccc(-c4cccc5ccccc45)cc3)c3ccc(-c4cccc5ccccc45)cc3)cc2)cc(-n2c3ccccc3c3ccccc32)c1]

A forward bias DC voltage was applied to the organic electroluminescent devices of Examples and Comparative Examples prepared as described above to measure electroluminescence (EL) characteristics with PR-650 of Photoresearch, and the T95 lifespan was measured using a lifespan measuring device manufactured by McScience at 500 cd/m² standard luminance. The measurement results are shown in Table 7.

TABLE 7

| | | | Current | | | |
|---|---|---|---|---|---|---|
| | | | Density | Brightness | Efficiency | |
| | Compound | Voltage | (mA/cm²) | (cd/m²) | (cd/A) | T(95) |
| comparative | — | 5.7 | 14.3 | 500.0 | 3.5 | 52.4 |
| example(1) | | | | | | |
| comparative | comparative | 5.0 | 9.6 | 500.0 | 5.2 | 88.7 |
| example(2) | compound A | | | | | |
| comparative | comparative | 5.1 | 9.4 | 500.0 | 5.3 | 91.1 |
| example(3) | compound B | | | | | |
| example(1) | P-3 | 4.5 | 7.9 | 500.0 | 6.3 | 120.0 |
| example(2) | P-5 | 4.7 | 9.1 | 500.0 | 5.5 | 115.7 |
| example(3) | P-8 | 4.7 | 9.1 | 500.0 | 5.5 | 115.9 |
| example(4) | P-9 | 4.7 | 9.3 | 500.0 | 5.4 | 115.1 |
| example(5) | P-13 | 4.7 | 9.1 | 500.0 | 5.5 | 112.4 |
| example(6) | P-14 | 4.8 | 8.9 | 500.0 | 5.6 | 113.0 |
| example(7) | P-22 | 4.8 | 8.6 | 500.0 | 5.8 | 114.1 |
| example(8) | P-30 | 4.9 | 9.1 | 500.0 | 5.5 | 112.1 |
| example(9) | P-38 | 4.6 | 8.2 | 500.0 | 6.1 | 117.2 |
| example(10) | P-39 | 4.0 | 8.6 | 500.0 | 5.8 | 113.8 |
| example(11) | P-47 | 4.6 | 8.1 | 500.0 | 6.2 | 117.8 |
| example(12) | P-51 | 4.6 | 8.1 | 500.0 | 6.2 | 118.0 |
| example(13) | P-52 | 4.8 | 8.9 | 500.0 | 5.6 | 114.6 |
| example(14) | P-53 | 4.5 | 8.1 | 500.0 | 6.2 | 119.2 |
| example(15) | P-58 | 4.5 | 7.9 | 500.0 | 6.3 | 118.7 |
| example(16) | P-69 | 4.8 | 8.8 | 500.0 | 5.7 | 113.5 |
| example(17) | P-72 | 4.6 | 8.2 | 500.0 | 6.1 | 117.5 |
| example(18) | P-77 | 4.7 | 9.4 | 500.0 | 5.3 | 114.8 |
| example(19) | P-83 | 4.6 | 8.3 | 500.0 | 6.0 | 120.3 |
| example(20) | P-86 | 4.6 | 8.3 | 500.0 | 6.0 | 119.6 |

As can be seen from the results of Table 7, when a blue organic light emitting device is manufactured using the material for an organic light emitting device of the present invention as the material for the emitting auxiliary layer, it is possible to improve the driving voltage, luminous efficiency and lifespan of the organic light emitting device compared to Comparative Example using Comparative Compound A and Comparative Compound B, which do not form an emitting auxiliary layer or have a basic skeleton similar to the compound of the present invention. In other words, in the case of Comparative Example 2 and Comparative Example 3 using the emitting auxiliary layer using Comparative Compound A and Comparative Compound B compared to Comparative Example 1 in which the emitting auxiliary layer was not formed, the driving voltage, efficiency, and lifespan of the device were improved, and When the compound of the present invention is used as the material for the emitting auxiliary layer, compared to the case where one of Comparative Examples 2 and 3 was used as the emitting auxiliary layer, the driving voltage of the organic light emitting device is generally lowered, and the luminous efficiency and especially the lifespan are remarkably improved.

Comparative Compound A and Comparative Compound B are the same as the compounds of the present invention in that one side of the tertiary amine compound is substituted with 9-carbazole and the other two moieties are substituted with phenyl-naphthyl, but are different from the compounds of the present invention in that carbazole is substituted at the para or meta position based on the substituted biphenyl.

As such, it can be confirmed that the device results are improved despite the fact that Comparative Compound A, Comparative Compound B and the compounds of the present invention are composed of similar components. In particular, there is a significant difference in the lifespan of the device, this is because the carbazole moiety of the compound of the present invention is twisted more than the comparative compounds in being substituted at the ortho position, so the intermolecular distance is increased and the Tg value is lowered, and as a result, it is possible to sufficiently deposit on the device even at a low temperature, and to have a great effect on the lifespan of the element.

Among the compounds of the present invention, when 2 phenyl-naphthyls substituted for tertiary amines in Formula 1 are identical to each other, it can be confirmed that the lifespan is increased more than when they are not.

Besides, the data measured using the DFT method (B3LYP/6-31g (D)) of the Gaussian program for Comparative Compound A, Comparative Compound B, and Compound P-3 of the present invention are shown in Table 8.

TABLE 8

| | Comparative | Comparative | |
|---|---|---|---|
| | compound A | compound B | P-3 |
| HOMO(eV) | −5.041 | −5.075 | −4.993 |

In Table 8, it can be seen that the HOMO level of the compound P-3 of the present invention is higher than that of Comparative Compound A and Comparative Compound B, and due to this difference, the hole transport from the hole transport layer to the emitting layer is faster and smoother, so the charge balance is improved, as a result, since interfacial light emitting between the emitting auxiliary layer and the host is reduced, and light emitting is mainly made in the host, it is determined that the overall performance of the element is affected. These results show that even for compounds with similar molecular skeletons, such as Comparative Compound A and Comparative Compound B and the compound of the present invention, depending on the substitution position of the substituent, the properties of the compound such as hole characteristics, light efficiency characteristics, energy level (HOMO, LUMO), hole injection and mobility characteristics, charge balance of holes and electrons, etc. may vary, and as a result, the result of the device can be remarkably derived to the extent that it is difficult to predict.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device having excellent element characteristics of high luminance, high light emission and long lifespan, and thus there is industrial applicability.

1. A compound represented by Formula 1: 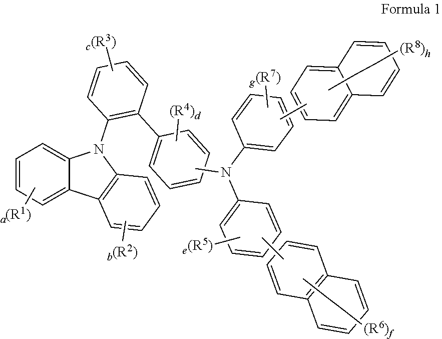
wherein:
1) R¹, R², R³, R⁴, R⁵ and R⁷ are each independently the same as or different from each other, and are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a C₂ -C₆₀ heterocyclic group including at least one heteroatom of O, N, S, Si or P; a fused ring group of a C₃-C₆₀ aliphatic ring and a C₆-C₆₀ aromatic ring; or in case a, b, c, d, e and g are 2 or more, a plurality of adjacent R¹s, or a plurality of R²s, or a plurality of R³s, or a plurality of R⁴s, or a plurality of R⁵s, or a plurality of R⁷s may be bonded to each other to form a ring,
2) R⁶ and R⁸ are each independently the same as or different from each other, and are each independently selected from the group consisting of hydrogen; deuterium; halogen; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; fluorenyl group; a fused ring group of a C₃-C₆₀ aliphatic ring and a C₆-C₆₀ aromatic ring; or in case f and h are 2 or more, a plurality of adjacent R⁶ s, or a plurality of R⁸ s may be bonded to each other to form a ring,
3) a, b, c, d, e and g are each independently integers from 0 to 4, h and f are independently integers from 0 to 7,
4) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound of claim 1, wherein the compound represented by Formula 1 is represented by any one of Formulas 1-1 to 1-3: 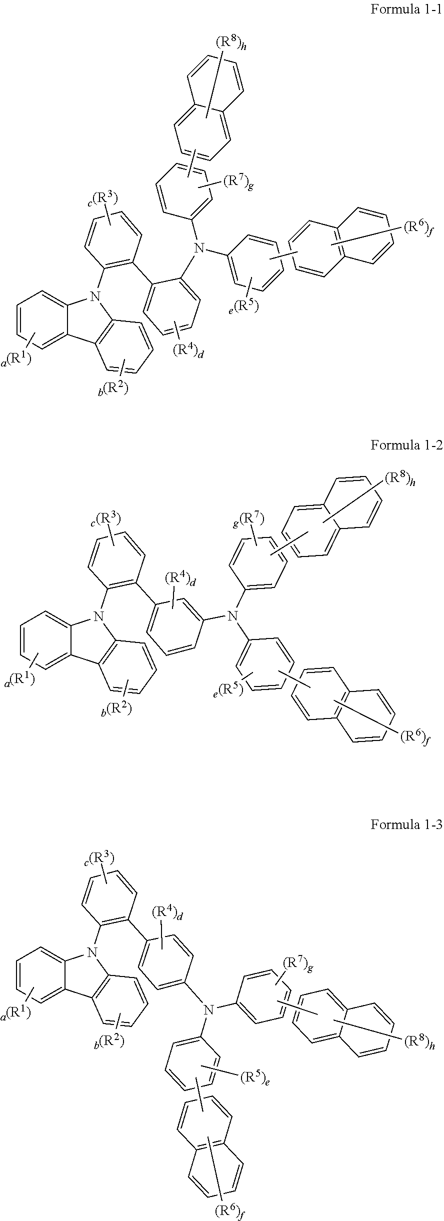
wherein R¹, R², R³, R⁴, R⁵, R⁶, R⁷, R⁸, a, b, c, d, e, f, g and h are the same as defined in claim 1.

3. The compound of claim 1, wherein the compound represented by Formula 1 is represented by any one of Formulas 1-4 to 1-6: 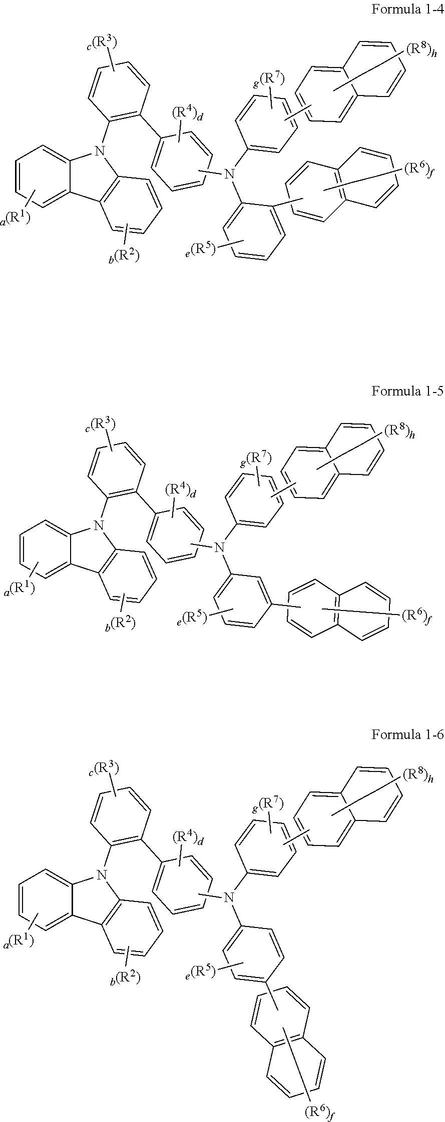
wherein R¹, R², R³, R⁴, R⁵, R⁶, R⁷, R⁸, a, b, c, d, e, f, g and h are the same as defined in claim 1.

4. The compound of claim 1, wherein the compound represented by Formula 1 is represented by Formula 1-7 or 1-8: 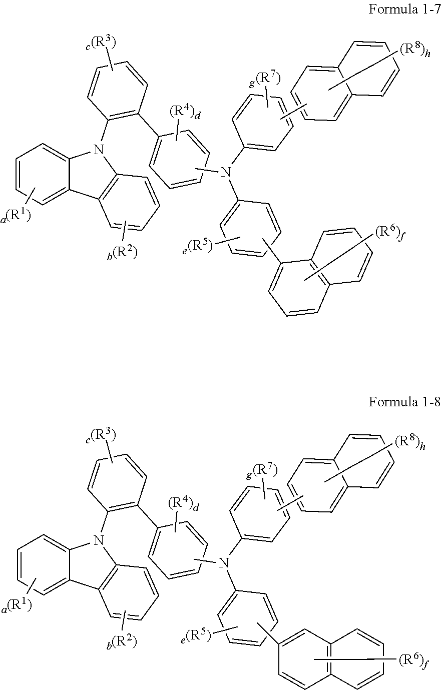
wherein R¹, R², R³, R⁴, R⁵, R⁶, R⁷, R⁸, a, b, c, d, e, f, g and h are the same as defined in claim 1.

5. The compound of claim 1, wherein the compound represented by Formula 1 is represented by Formula 1-9: 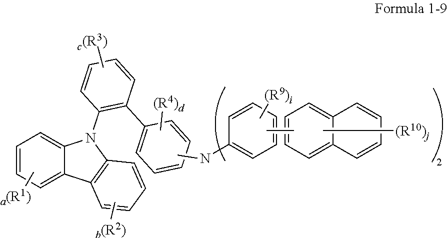
wherein:
1) R¹, R², R³, R⁴, a, b, c and d are the same as defined in claim 1,
2) R⁹ is the same as the definition of R¹ in claim 1,
3) R¹⁹ is the same as the definition of R⁶ in claims 1, and
4) i is an integer of 0 to 4, j is an integer of 0 to 7.

6. The compound of claim 1, wherein the compound represented by Formula 1 is represented by any one of Formulas 1-10 to 1-12: 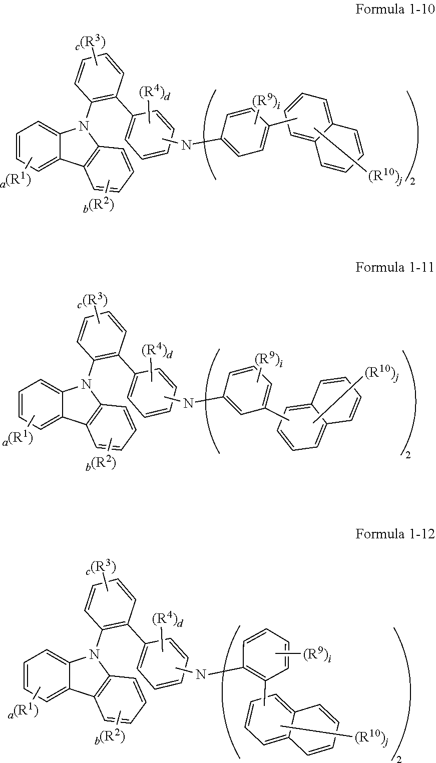
wherein:
1) R¹, R², R³, R⁴, a, b, c and d are the same as defined in claim 1, and
2) R⁹ is the same as the definition of R¹ in claim 1,
3) R¹⁰ is the same as the definition of R⁶ in claims 1, and
4) i is an integer of 0 to 4, j is an integer of 0 to 7.

7. The compound of claim 1, wherein the compound represented by Formula 1 is any one of following compounds P-1 to P-90:

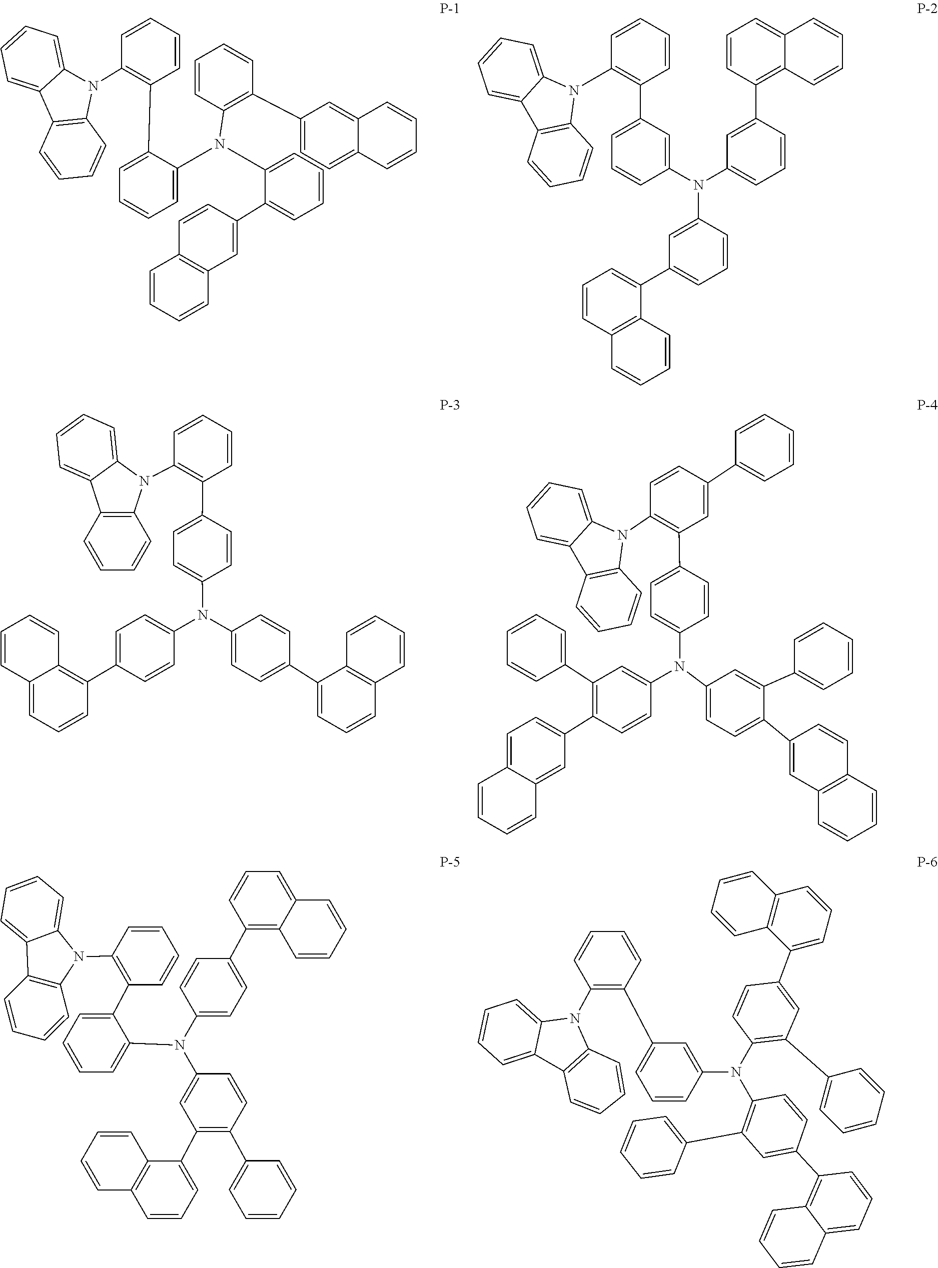

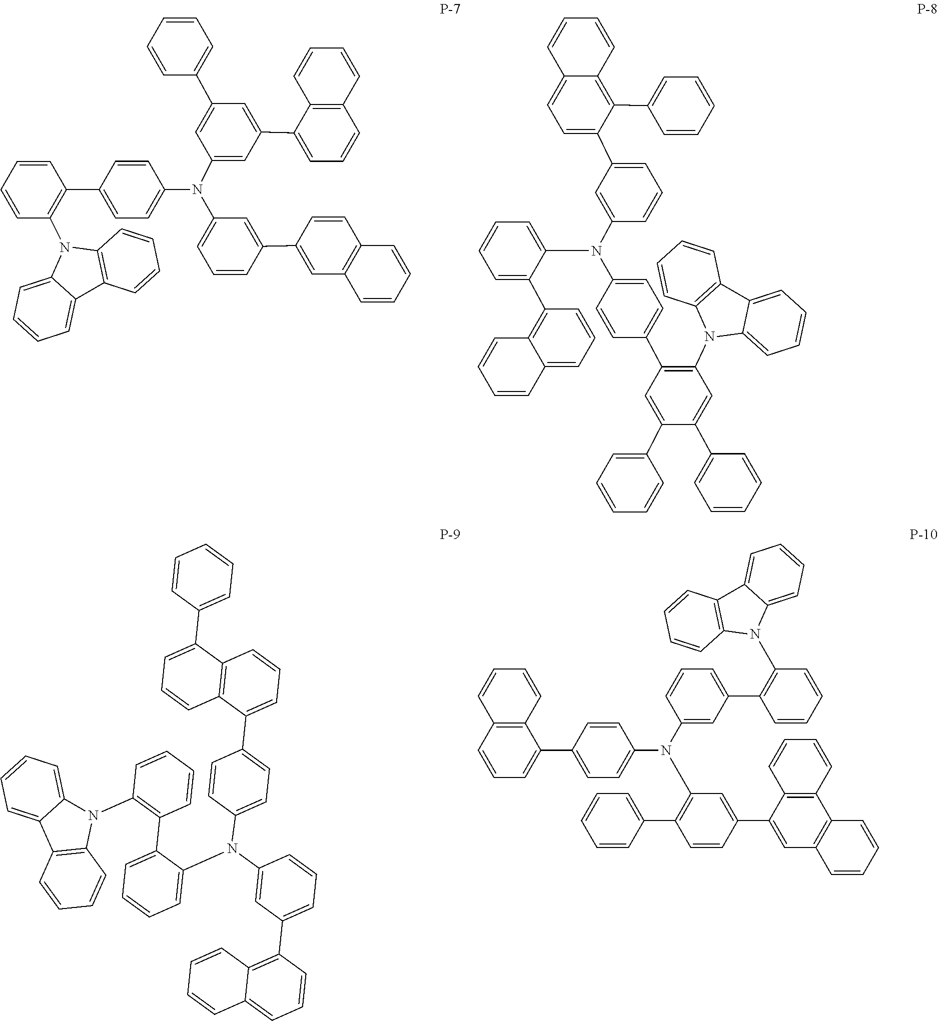

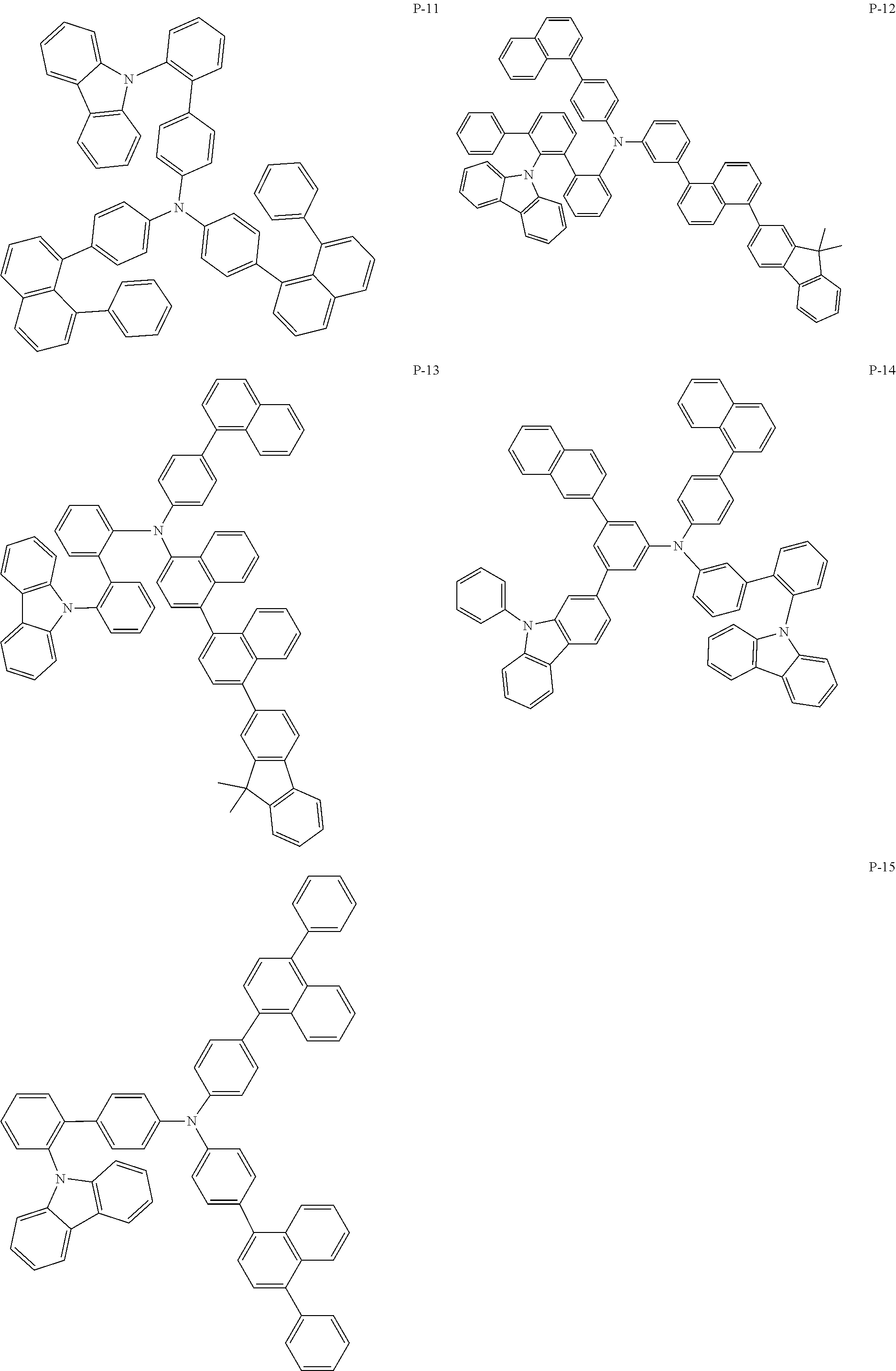

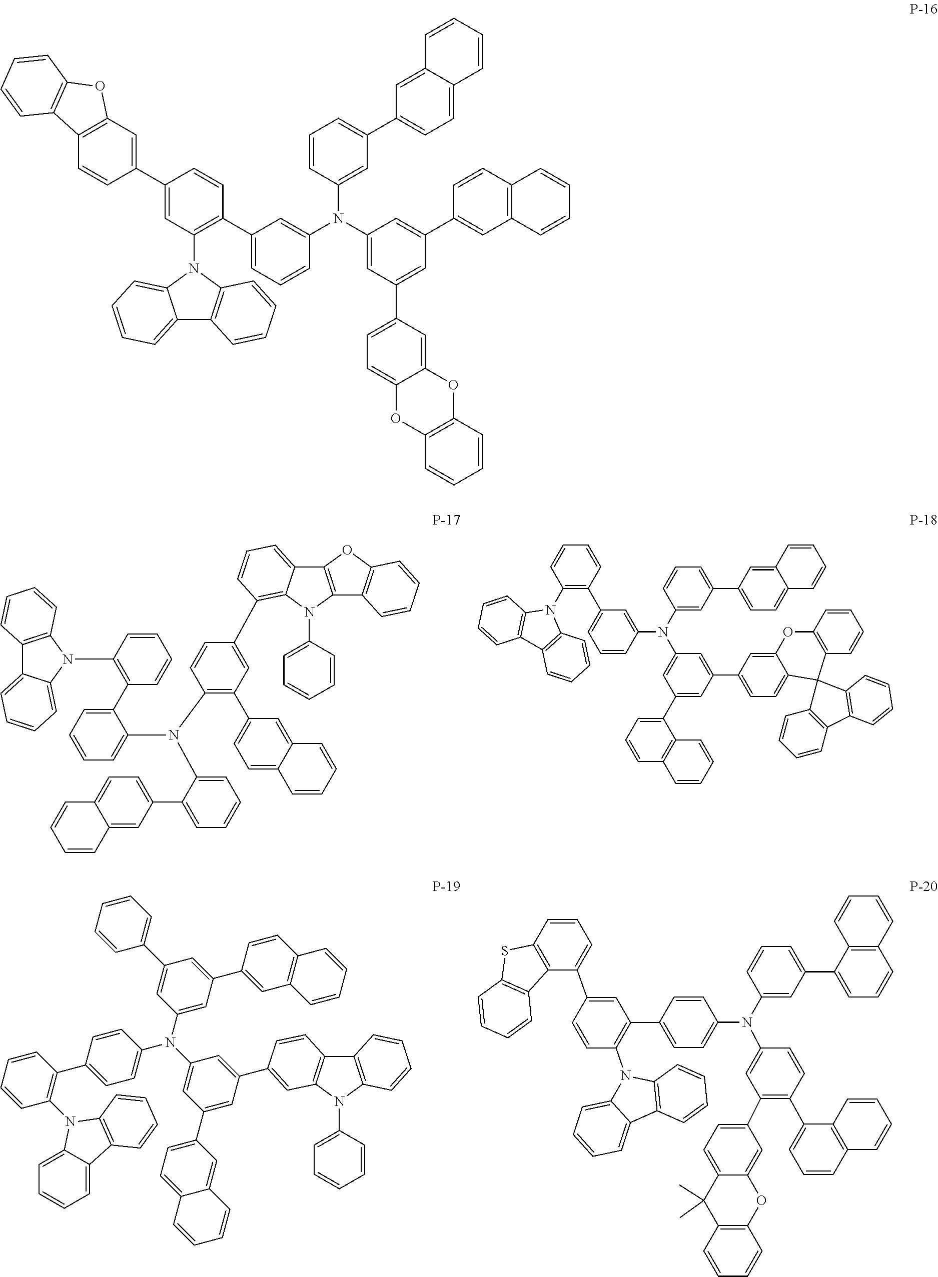

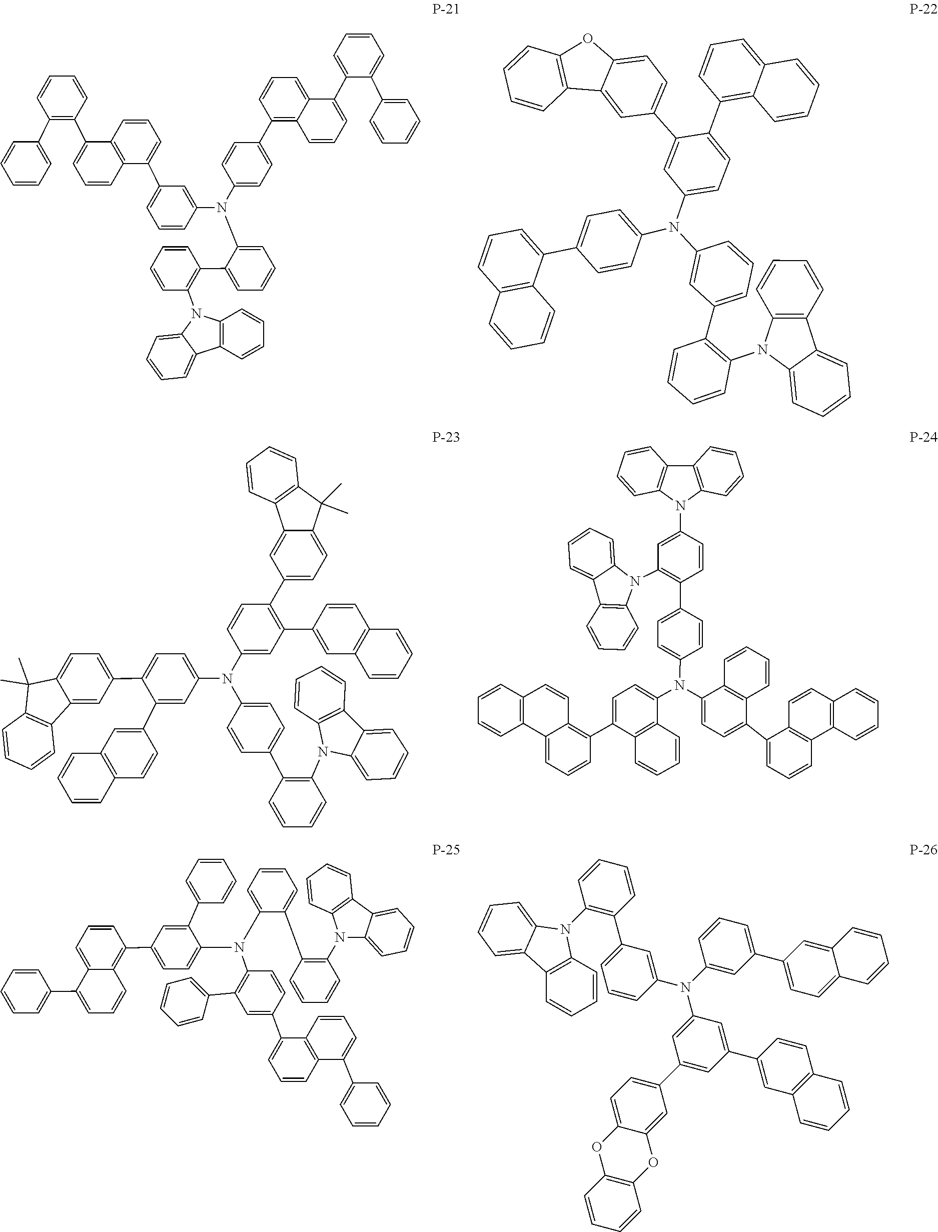

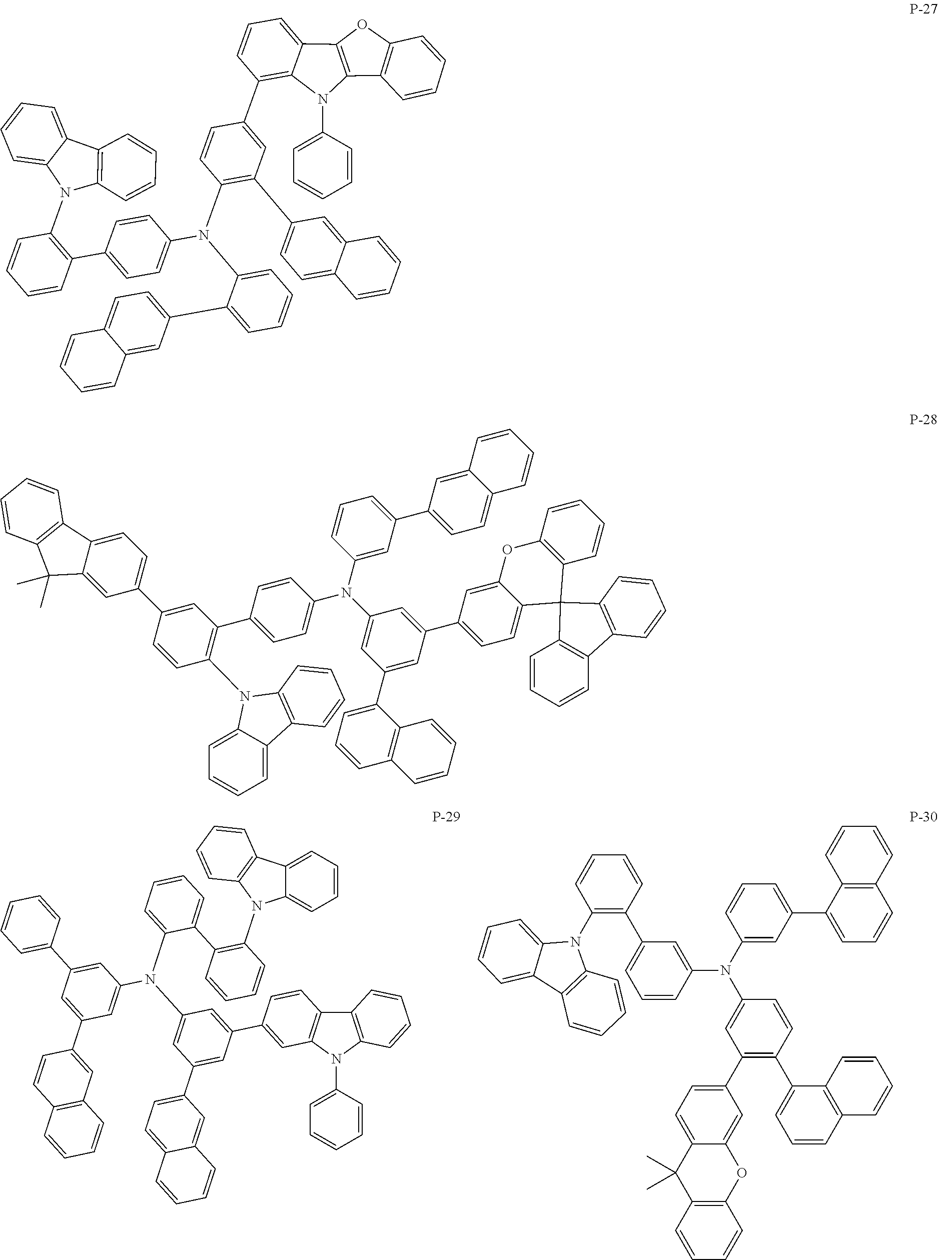

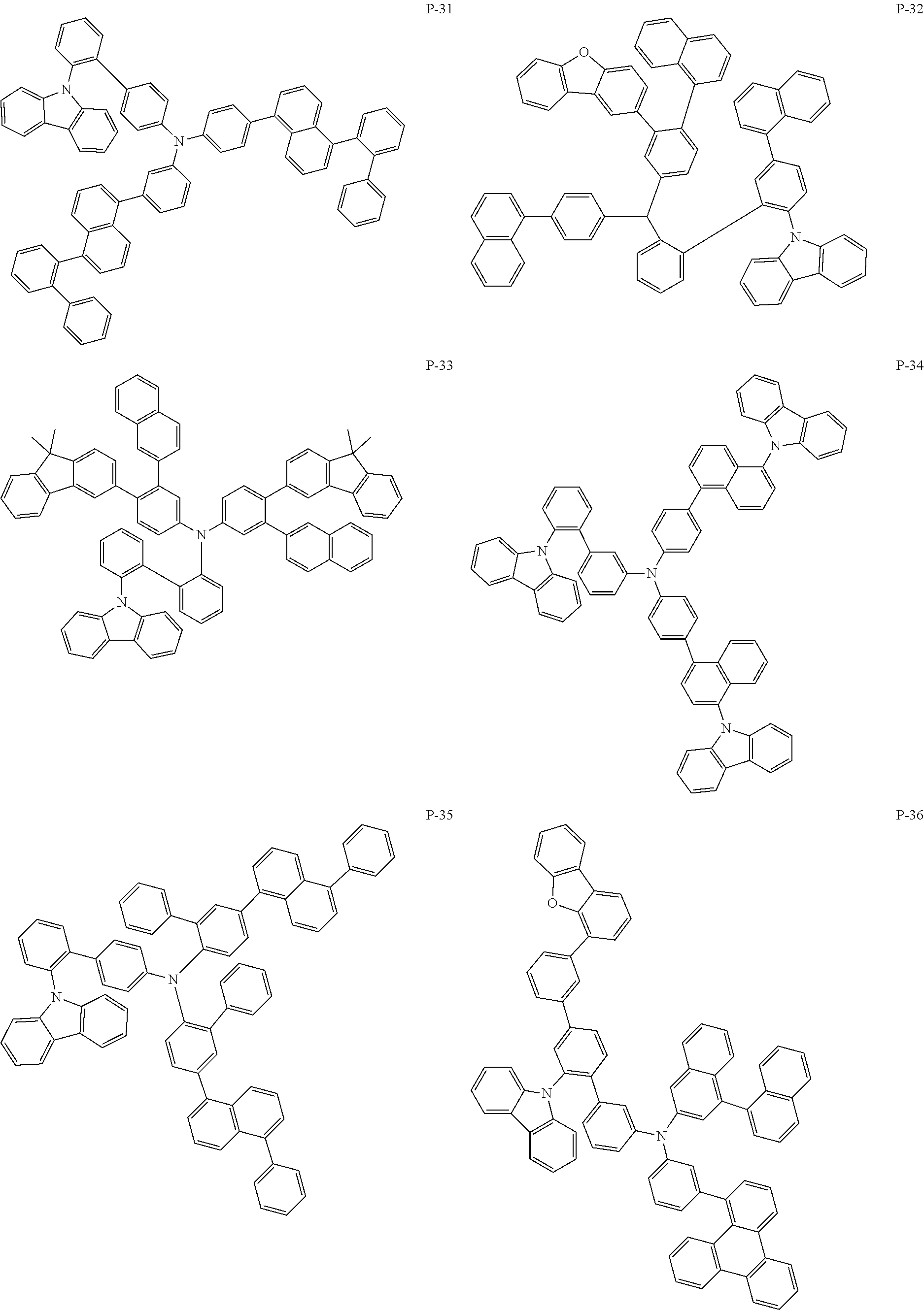

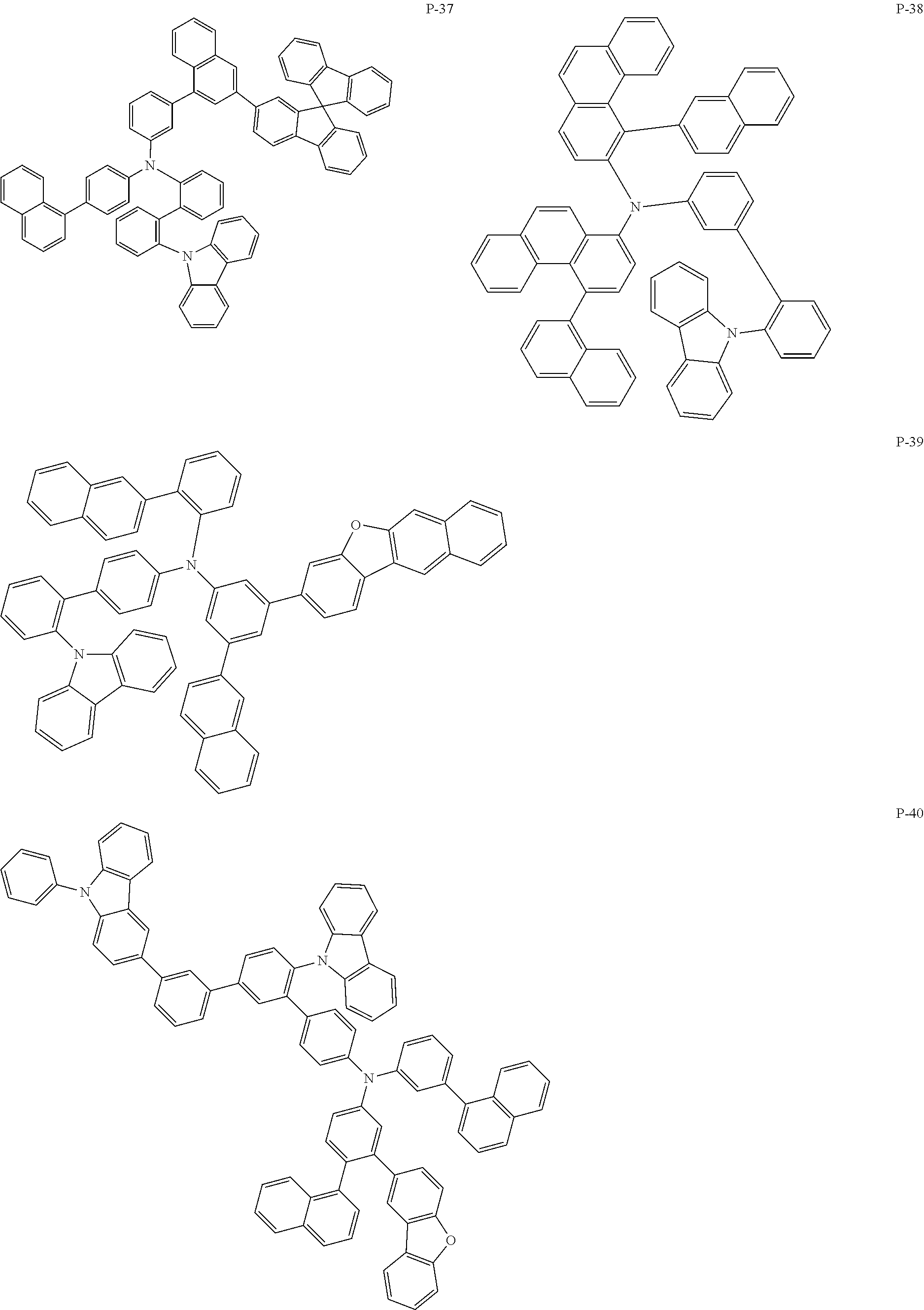

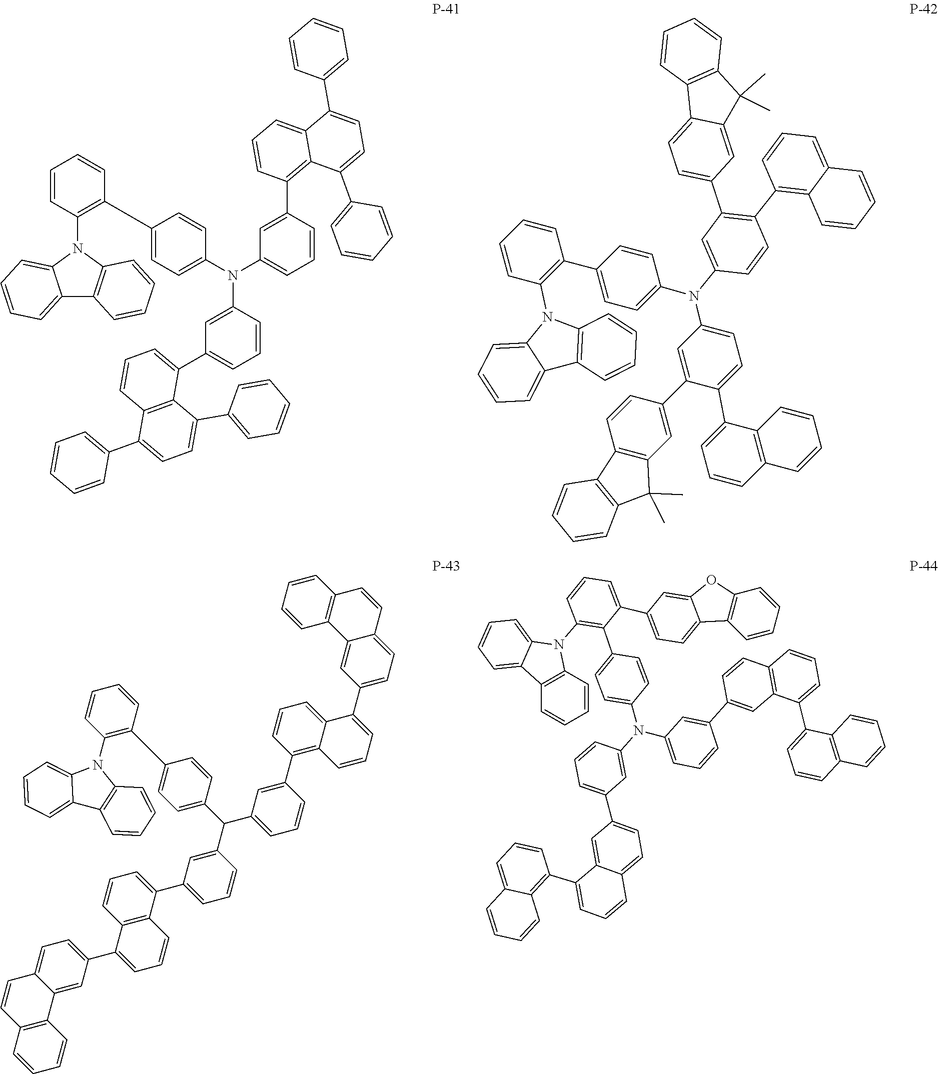

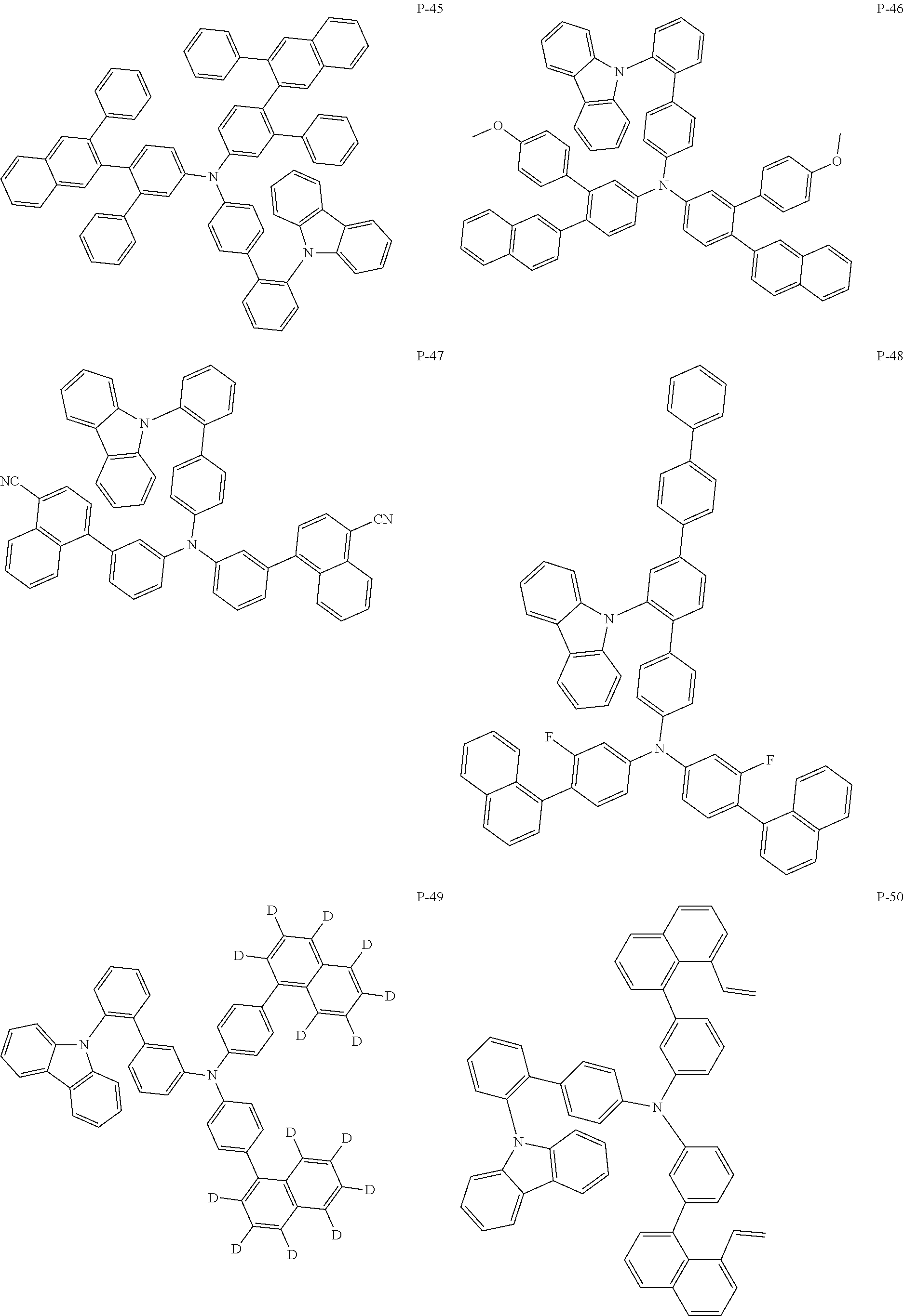

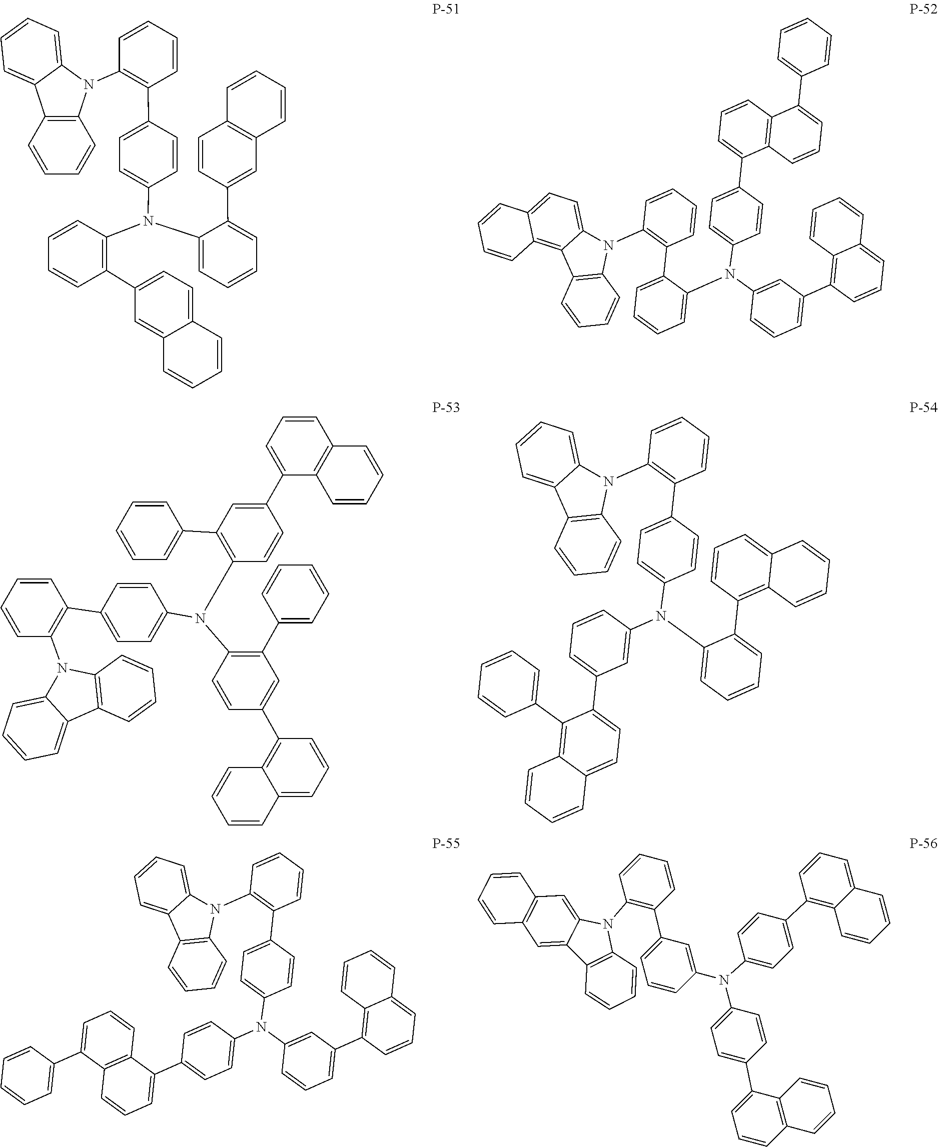

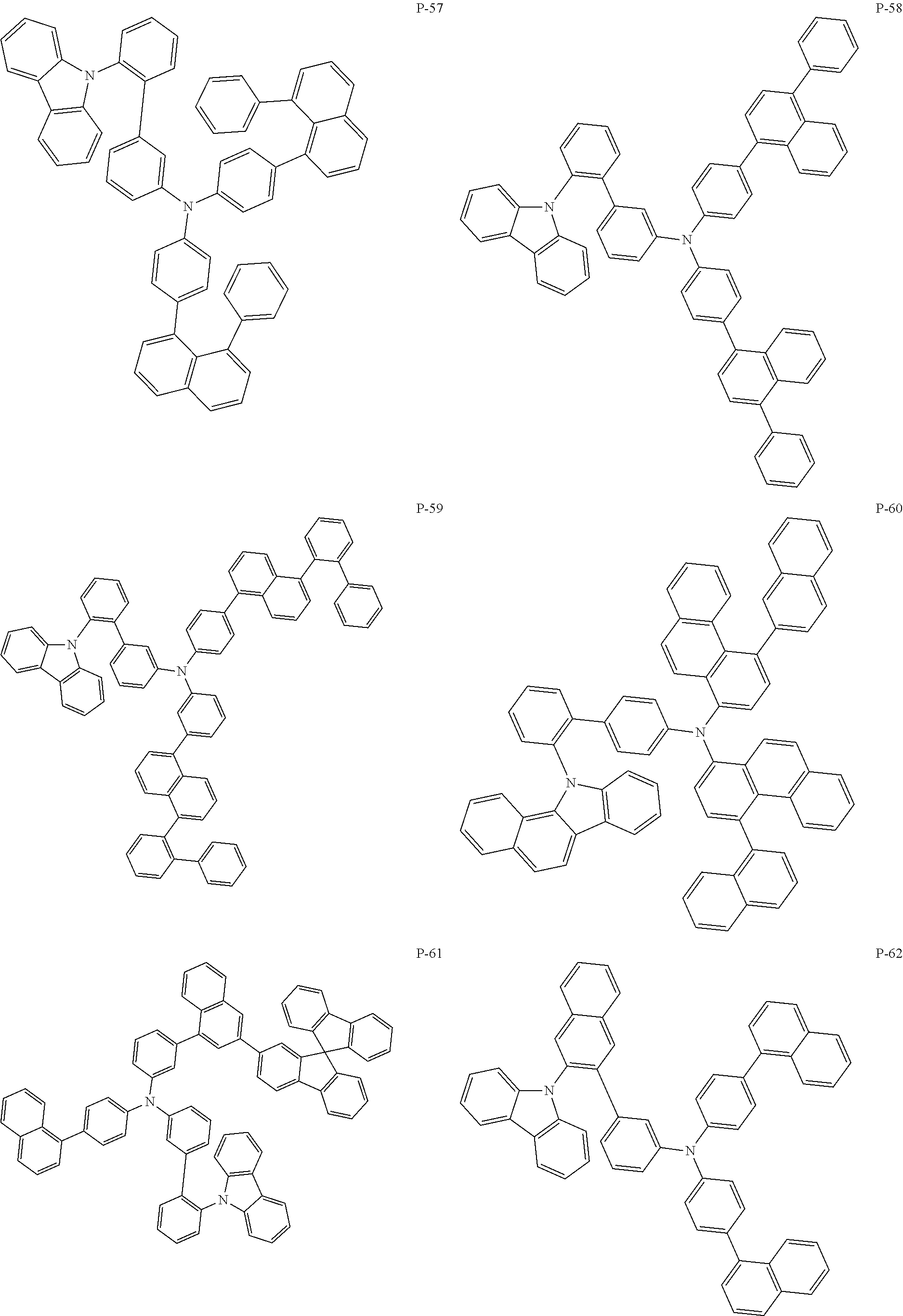

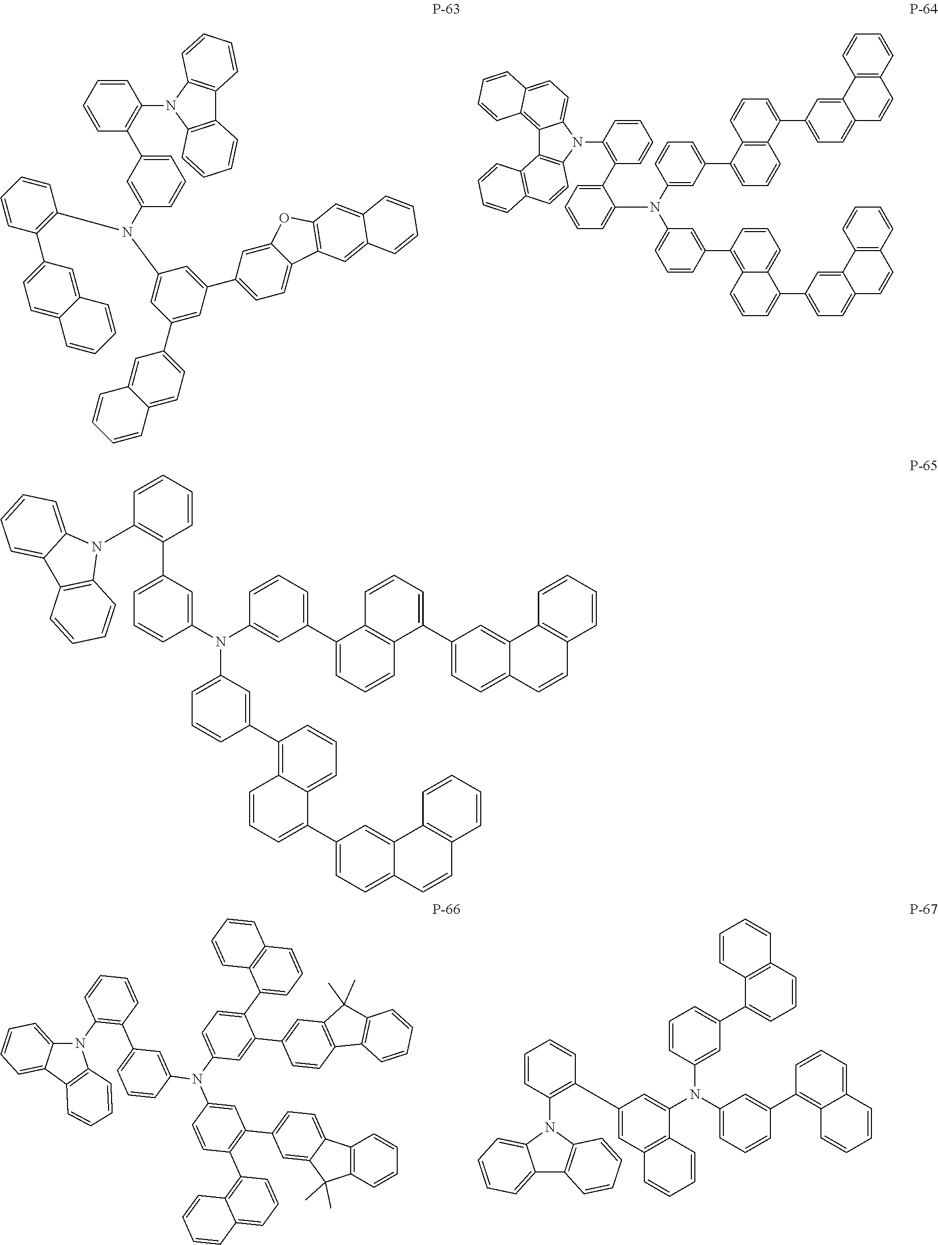

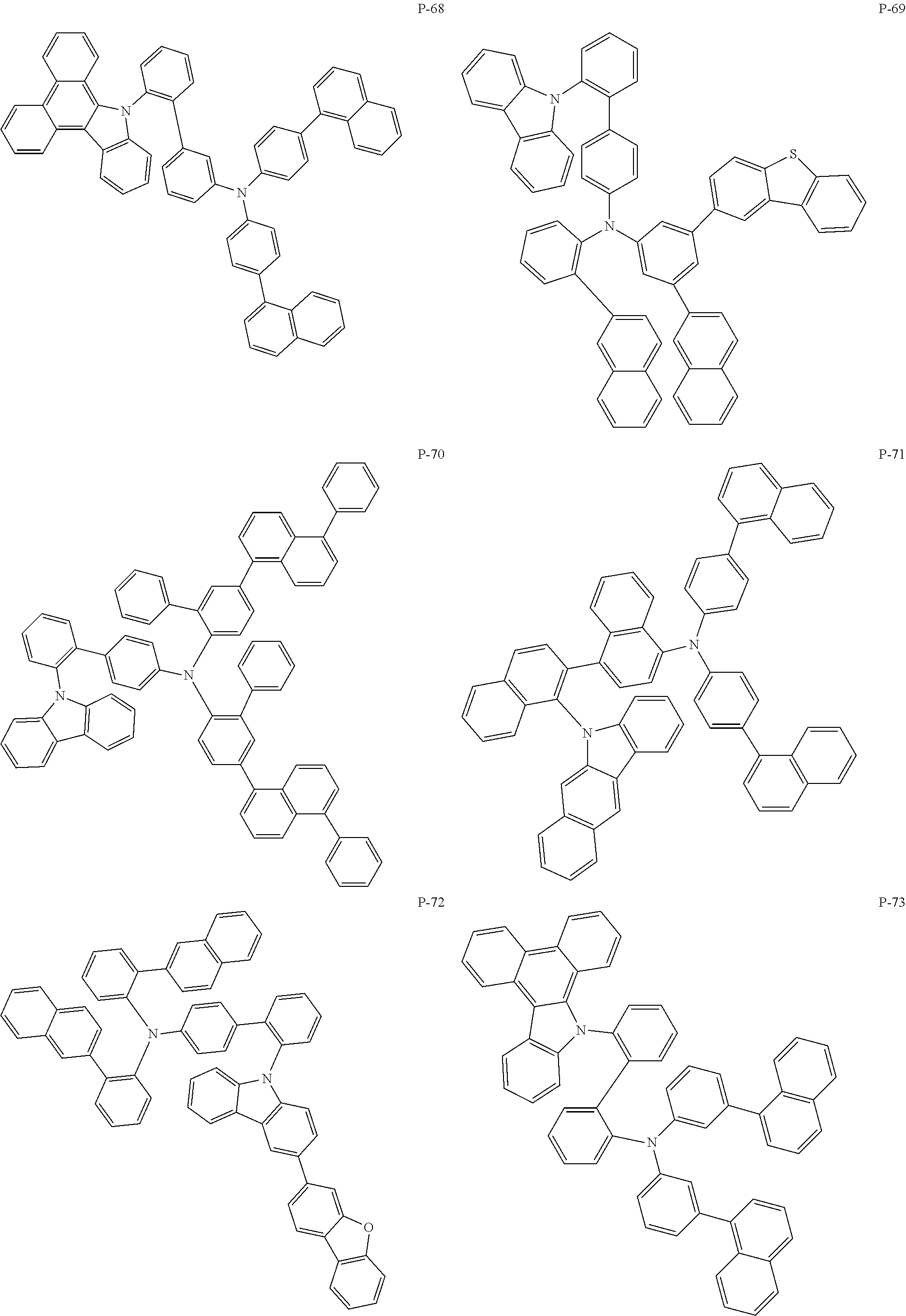

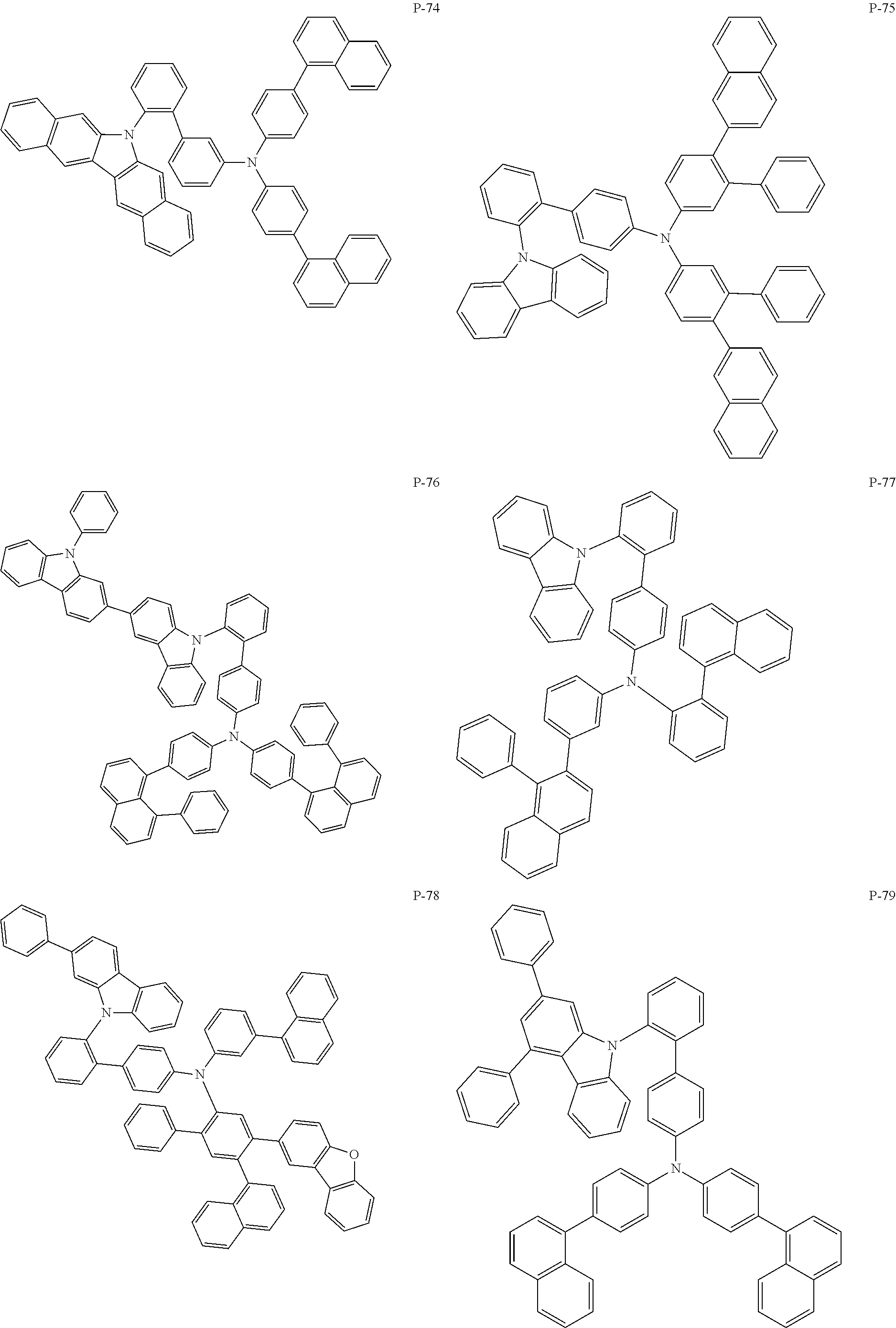

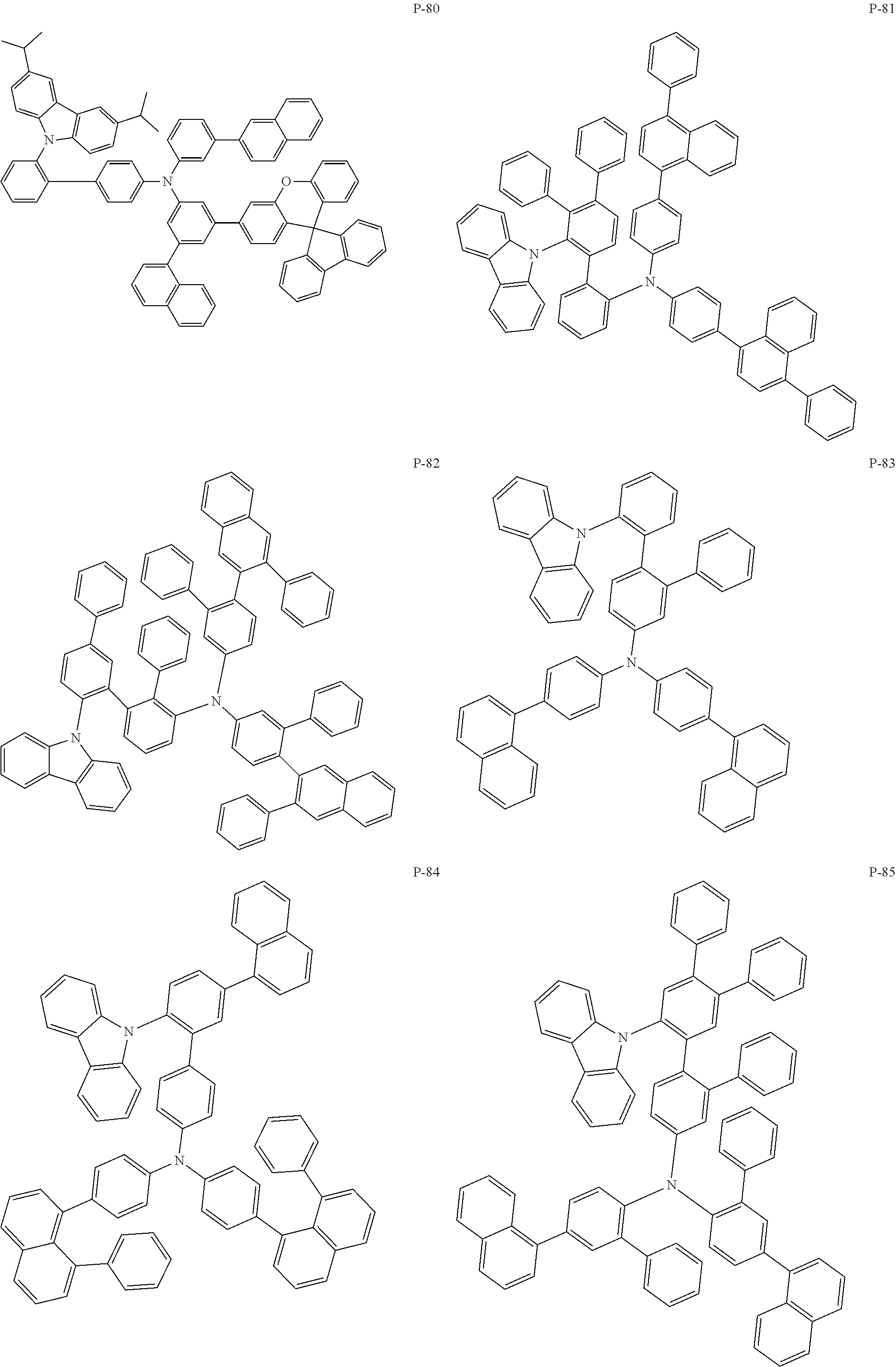

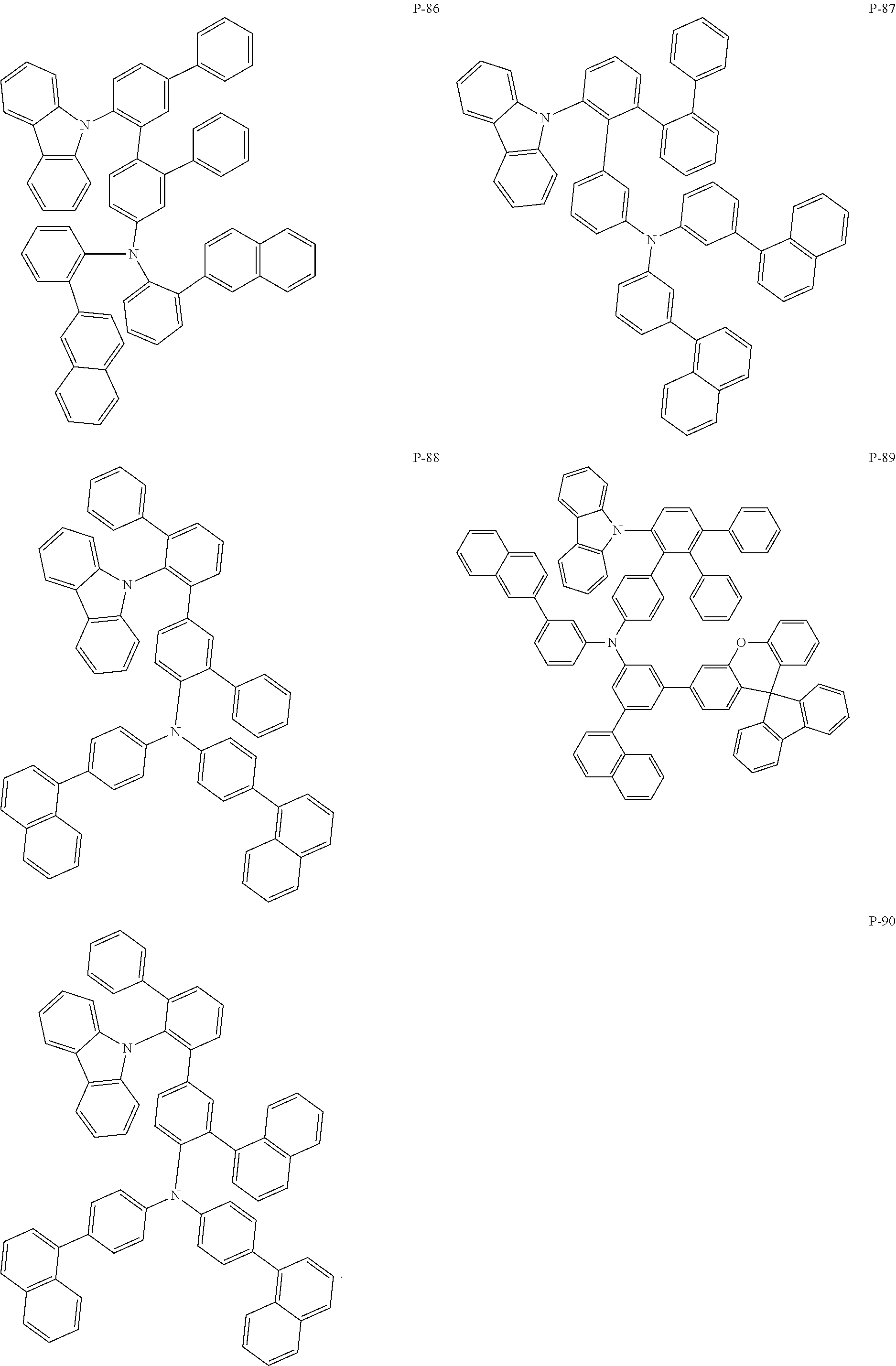

8. An organic electronic element comprising a first electrode, a second electrode, and an organic material layer formed between the first electrode and the second electrode, wherein the organic material layer may include a hole transport layer; an emitting layer; and an emitting auxiliary layer formed between the hole transport layer and the emitting layer; wherein the emitting auxiliary layer comprises the compound represented by Formula 1 of claim 1.

9. The organic electronic element of claim 8, wherein the emitting layer comprises a compound represented by any one of Formulas 2 to 4: 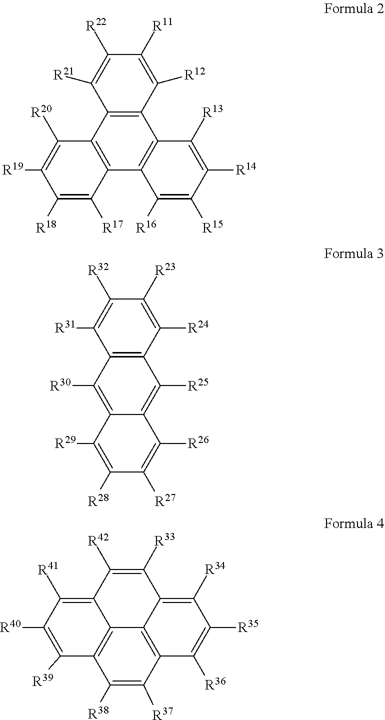
wherein:
1) R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹ and R⁴² are each independently selected from the group consisting of hydrogen; deuterium; a C₆-C₆₀ aryl group; a C₂-C₆₀ heterocyclic group including at least one heteroatom of O, N, S, Si or P; a C₁-C₅₀ alkyl group; a C₁-C₅₀ alkoxy group; a C₂-C₂₀ alkenyl group; and -L′-NR^{a}R^{b}; or alternatively, adjacent groups may be bonded to each other to form a ring,
2) L′ is selected from the group consisting of a single bond; a C₆-C₆₀ arylene group; fluorenylene group; a C₂-C₆₀ heterocyclic group including at least one heteroatom of O, N, S, Si or P; a C₃-C₆₀ aliphatic ring;
3) wherein R^{a} and R^{b} are each independently selected from the group consisting of a C₆˜C₆₀ aryl group; fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring.

10. The organic electronic element of claim 9, wherein a compound represented by Formula 2 is represented by any one of the following compounds 8-1 to 8-18:

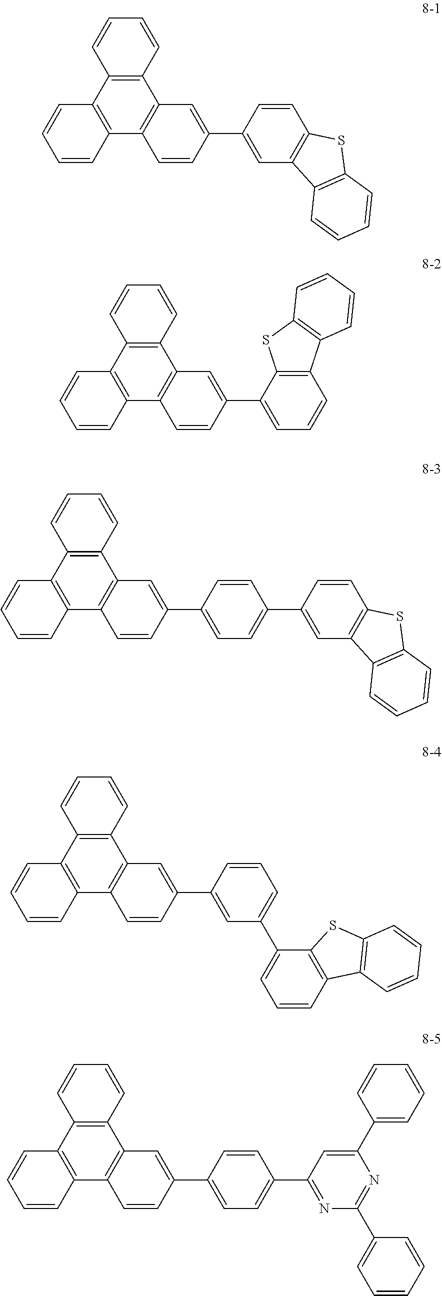

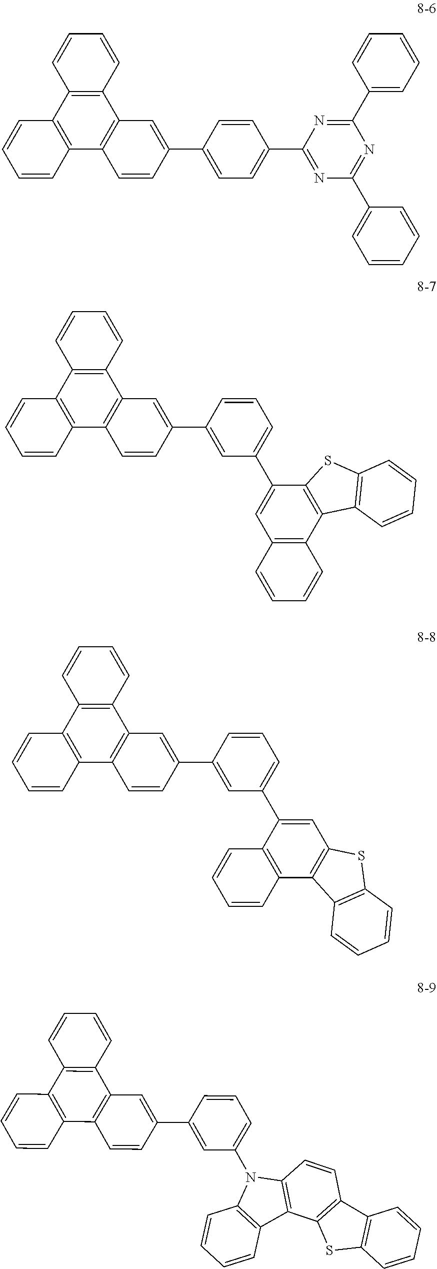 [SMILES: c1ccc(-c2nc(-c3ccccc3)nc(-c3ccc(-c4ccc5c6ccccc6c6ccccc6c5c4)cc3)n2)cc1 ; c1cc(-c2ccc3c4ccccc4c4ccccc4c3c2)cc(-c2cc3ccccc3c3c2sc2ccccc23)c1 ; c1cc(-c2ccc3c4ccccc4c4ccccc4c3c2)cc(-c2cc3sc4ccccc4c3c3ccccc23)c1 ; c1cc(-c2ccc3c4ccccc4c4ccccc4c3c2)cc(-n2c3ccccc3c3c4sc5ccccc5c4ccc32)c1]

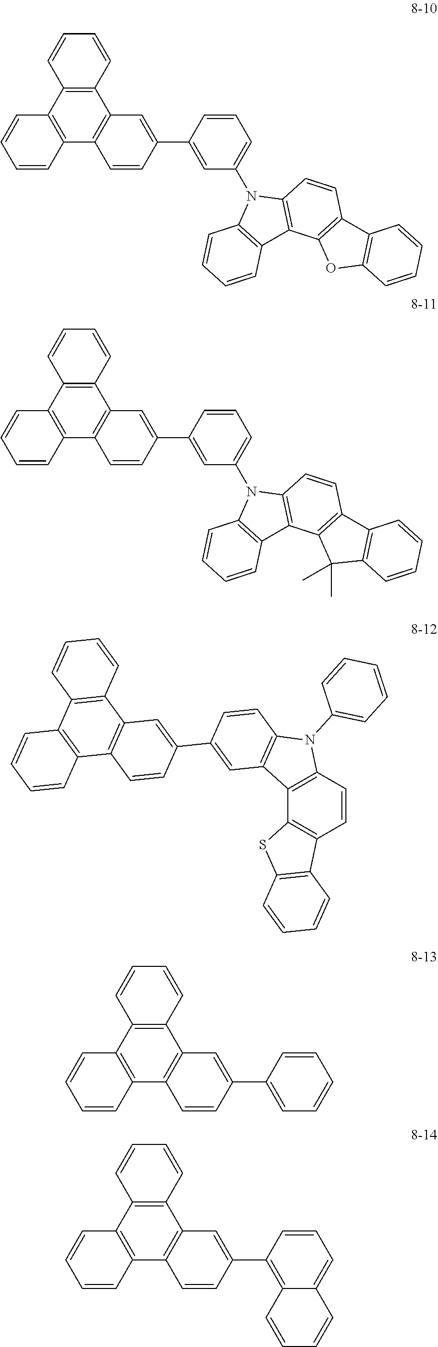

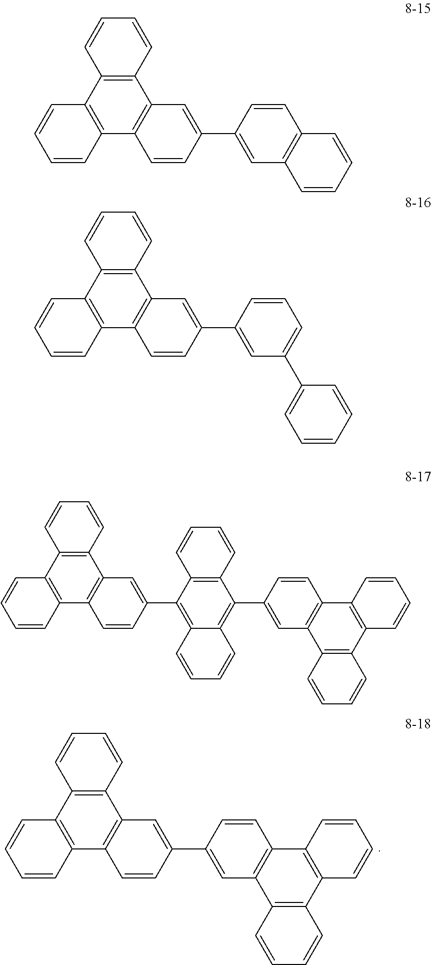

11. The organic electronic element of claim 9, wherein a compound represented by Formula 3 is represented by any one of the following compounds 9-1 to 9-32:

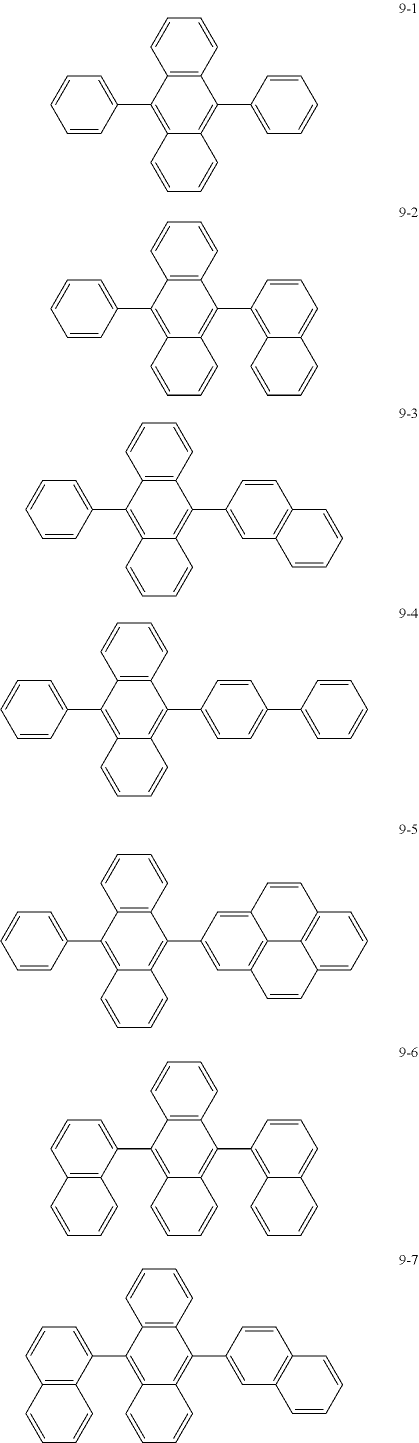

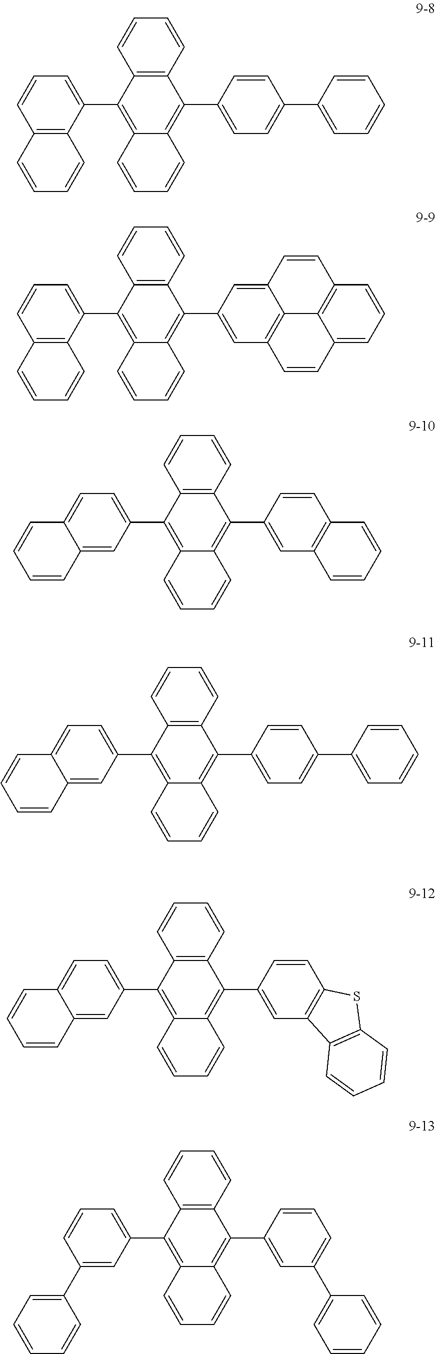

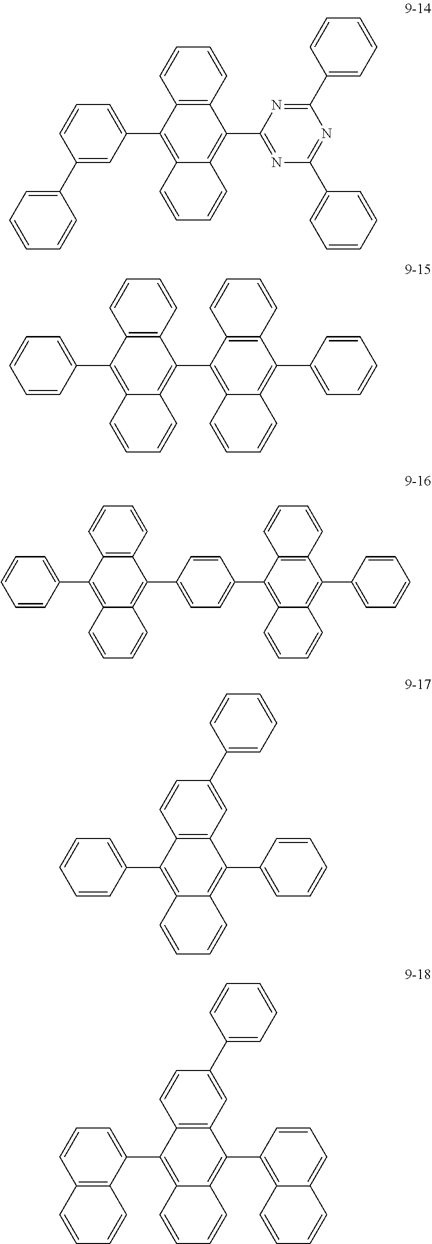

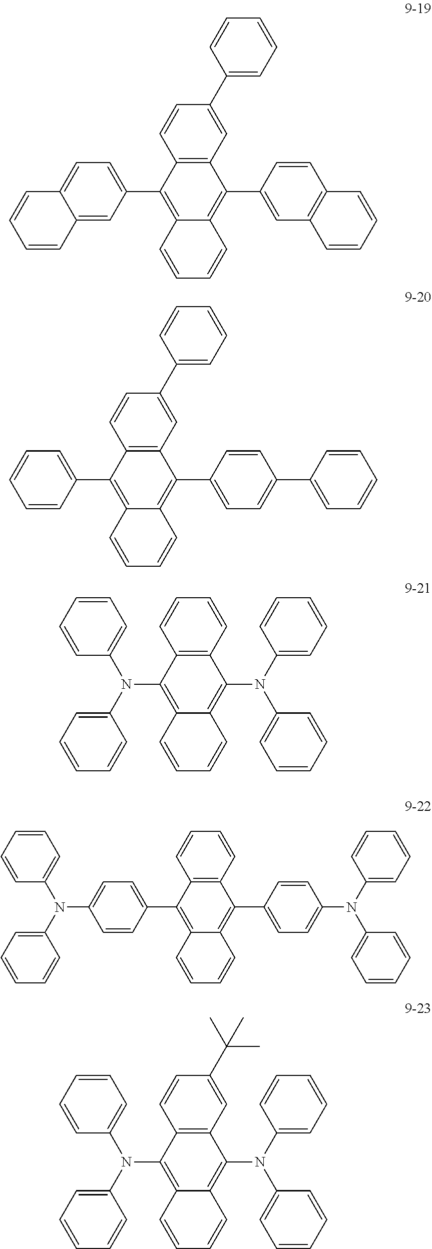

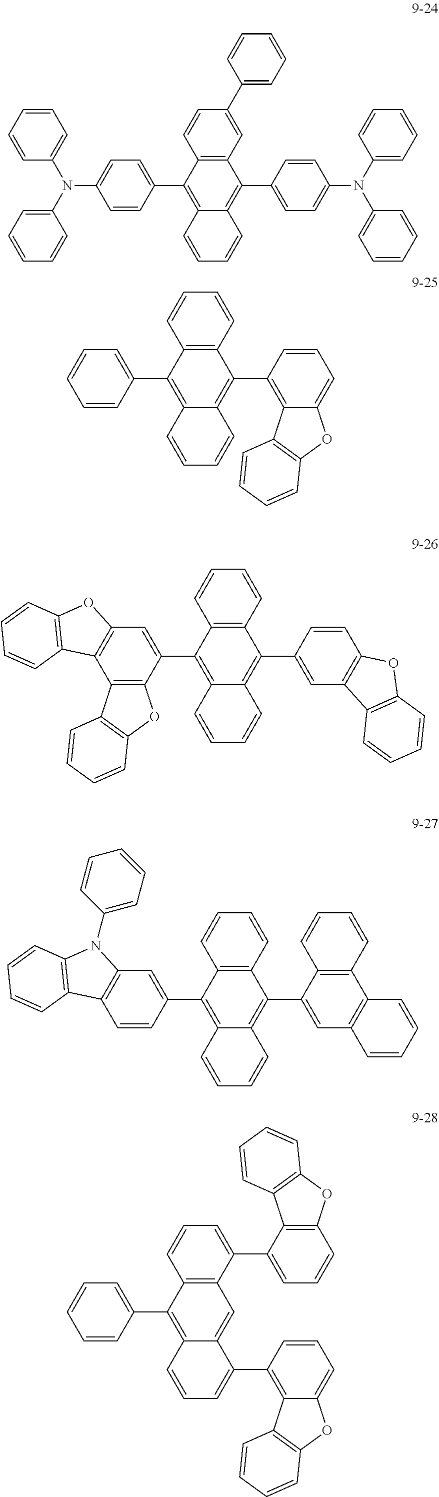

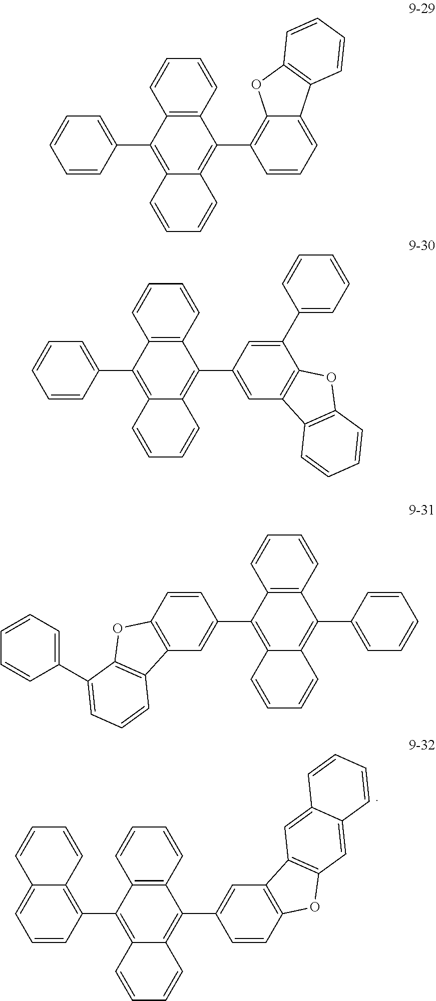 [SMILES: c1ccc(-c2c3ccccc3c(-c3cccc4c3oc3ccccc34)c3ccccc23)cc1 ; c1ccc(-c2c3ccccc3c(-c3cc(-c4ccccc4)c4oc5ccccc5c4c3)c3ccccc23)cc1 ; c1ccc(-c2c3ccccc3c(-c3ccc4oc5c(-c6ccccc6)cccc5c4c3)c3ccccc23)cc1 ; c1ccc2cc3c(cc2c1)oc1ccc(-c2c4ccccc4c(-c4cccc5ccccc45)c4ccccc24)cc13]

12. The organic electronic element of claim 9, wherein a compound represented by Formula 4 is represented by any one of the following compounds 10-1 to 10-20:

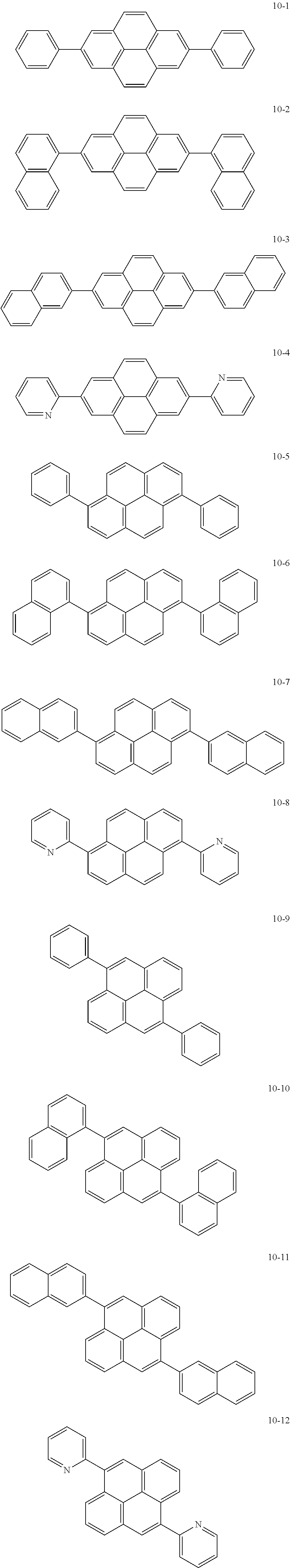

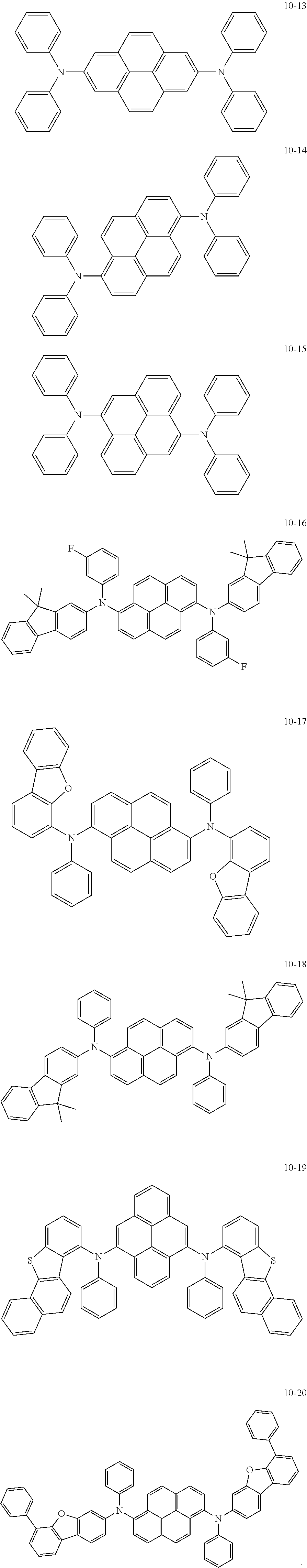

13. The organic electronic element of claim 8, further comprising a light efficiency enhancing layer formed on at least one surface of the first electrode and the second electrode, the surface being opposite to the organic material layer.

14. The organic electronic element of claim 8, wherein the organic material layer comprises 2 or more stacks each stack comprising a hole transport layer, an emitting layer, and an electron transport layer sequentially formed on the first electrode.

15. The organic electronic element of claim 8, wherein the organic material layer further comprises a charge generation layer formed between the 2 or more stacks.

16. An electronic device comprising a display device comprising the organic electronic element of claim 8; and a control unit for driving the display device.

17. The electronic device according to claim 16, wherein the organic electronic element is at least one of an OLED, an organic solar cell, an organic photo conductor (OPC), organic transistor (organic TFT) and an element for monochromic or white illumination.
